SEQUENCE LISTING (7 sequences)

SEQ ID NO: 1 - protein, 27 aa, Artificial sequence
  synthetic sequence
PPGKPQGPPPQGGNQPQGPPPPPGKPQ

SEQ ID NO: 2 - protein, 25 aa, Artificial sequence
  synthetic sequence
GNPQGPSPQGGNKPQGPPPPPGKPQ

SEQ ID NO: 3 - protein, 27 aa, Artificial sequence
  synthetic sequence
SPPGKPQGPPQQEGNKPQGPPPPGKPQ

SEQ ID NO: 4 - protein, 7 aa, Artificial sequence
  synthetic sequence
GGHPPPP

SEQ ID NO: 5 - protein, 7 aa, Artificial sequence
  synthetic sequence
ESPSLIA

SEQ ID NO: 6 - protein, 15 aa, Artificial sequence
  synthetic sequence
SKITHRIHWESASLL

SEQ ID NO: 7 - protein, 11 aa, Artificial sequence
  synthetic sequence
AHKSEVAHRFK

(19) United States
(12) Patent Application Publication
(10) Pub. No.: US 2023/0013771 A1
(43) Pub. Date: Jan. 19, 2023

(54) Method of Detecting an Analyte in a Sample

(71) Applicant: Essenlix Corp., Monmouth Junction, NJ (US)

(72) Inventor: Stephen Y. Chou, Princeton, NJ (US)

(21) Appl. No.: 17/187,498

(22) Filed: Feb. 26, 2021

Related U.S. Application Data
(63) Continuation of application No. 15/763,794, filed on Mar. 27, 2018, now abandoned.
(60) Provisional application No. 62/234,538, filed on Sep. 29, 2015.

(51) Int. Cl. G01N 33/53; G01N 21/64; G01N 21/65

(52) U.S. Cl. CPC G01N 33/5302; G01N 21/6452; G01N 21/658; G01N 2201/0221; G01N 21/6428; G01N 2021/6439; G01N 21/648

(57) ABSTRACT

A method for sample analysis that employs a signal-amplifying nanosensor is provided. An implementation of the present method may include a) obtaining a sample, b) applying the sample to a signal-amplifying nanosensor containing a capture agent that binds to an analyte of interest, under conditions suitable for binding of the analyte in a sample to the capture agent, c) washing the signal-amplifying nanosensor, and d) reading the signal-amplifying nanosensor, thereby obtaining a measurement of the amount of the analyte in the sample. In some embodiments, the analyte may be a biomarker, an environmental marker, or a foodstuff marker. Also provided herein are kits that find use in performing the present method.

### CROSS-REFERENCING

This application claims the benefit of provisional application serial nos. 62/234,538, filed on Sep. 29, 2015, which is incorporated herein in its entirety for all purposes.

### BACKGROUND

This application relates to a method of detecting analytes in a sample using luminescence signals. Detection of analytes in a sample is important in many applications, including diganostics, personalized medicine, environmental monitoring and food testing. However, many conventional methods for analyte detection require invasive sample collection procedures, a specialized sample handling facility for sample collection and processing, bulky and costly assay readers, and/or technical staff to analyze the samples, making the detection process time consuming, intrusive and/or expensive. Thus, there is a need for fast, non-invasive and cost-effective ways to detect analytes in a sample.

### SUMMARY

A method for sample analysis that employs a signal-amplifying nanosensor is provided. An implementation of the present method may include a) obtaining a sample, b) applying the sample to a signal-amplifying nanosensor containing a capture agent that binds to an analyte of interest, under conditions suitable for binding of the analyte in a sample to the capture agent, c) washing the signal-amplifying nanosensor, and d) reading the signal-amplifying nanosensor, thereby obtaining a measurement of the amount of the analyte in the sample. In some embodiments, the analyte may be a biomarker, an environmental marker, or a foodstuff marker. The sample in some instances is a liquid sample, and may be a diagnostic sample (such as saliva, serum, blood, sputum, urine, sweat, lacrima, semen, or mucus); an environmental sample obtained from a river, ocean, lake, rain, snow, sewage, sewage processing runoff, agricultural runoff, industrial runoff, tap water or drinking water; or a foodstuff sample obtained from tap water, drinking water, prepared food, processed food or raw food.

In any embodiment, the signal-amplifying nanosensor may be placed in a microfluidic device and the applying step b) may include applying a sample to a microfluidic device comprising the signal-amplifying nanosensor.

In any embodiment, the reading step d) may include detecting a fluorescence or luminescence signal from the signal-amplifying nanosensor.

In any embodiment, the reading step d) may include reading the signal-amplifying nanosensor with a handheld device configured to read the signal-amplifying nanosensor.

The handheld device may be a mobile phone, e.g., a smart phone.

In any embodiment, the signal-amplifying nanosensor may include a labeling agent that can bind to an analyte-capture agent complex on the signal-amplifying nanosensor.

In any embodiment, the present method may further include, between steps c) and d), the steps of applying to the signal-amplifying nanosensor a labeling agent that binds to an analyte-capture agent complex on the signal-amplifying nanosensor, and washing the signal-amplifying nanosensor.

In any embodiment, the reading step d) may include reading an identifier for the signal-amplifying nanosensor. The identifier may be an optical barcode, a radio frequency ID tag, or combinations thereof.

In any embodiment, the present method may further include applying a control sample to a control signal-amplifying nanosensor containing a capture agent that binds to the analyte, wherein the control sample includes a known detectable amount of the analyte, and reading the control signal-amplifying nanosensor, thereby obtaining a control measurement for the known detectable amount of the analyte in a sample.

In any embodiment, the sample may be a diagnostic sample obtained from a subject, the analyte may be a biomarker, and the measured amount of the analyte in the sample may be diagnostic of a disease or a condition.

In any embodiment, the present method may further include receiving or providing to the subject a report that indicates the measured amount of the biomarker and a range of measured values for the biomarker in an individual free of or at low risk of having the disease or condition, wherein the measured amount of the biomarker relative to the range of measured values is diagnostic of a disease or condition.

In any embodiment, the present method may further include diagnosing the subject based on information including the measured amount of the biomarker in the sample. In some cases, the diagnosing step includes sending data containing the measured amount of the biomarker to a remote location and receiving a diagnosis based on information including the measurement from the remote location.

In any embodiment, the biomarker may be selected from Tables 1, 2, 3 or 7. In some instances, the biomarker is a protein selected from Tables 1, 2, or 3. In some instances, the biomarker is a nucleic acid selected from Tables 2, 3 or 7. In some instances, the biomarker is an infectious agent-derived biomarker selected from Table 2. In some instances, the biomarker is a microRNA (miRNA) selected from Table 7.

In any embodiment, the applying step b) may include isolating miRNA from the sample to generate an isolated miRNA sample, and applying the isolated miRNA sample to the signal-amplifying nanosensor.

In any embodiment, the signal-amplifying nanosensor may contain a plurality of capture agents that each binds to a biomarker selected from Tables 1, 2, 3 and/or 7, wherein the reading step d) includes obtaining a measure of the amount of the plurality of biomarkers in the sample, and wherein the amount of the plurality of biomarkers in the sample is diagnostic of a disease or condition.

In any embodiment, the capture agent may be an antibody epitope and the biomarker may be an antibody that binds to the antibody epitope. In some embodiments, the antibody epitope includes a biomolecule, or a fragment thereof, selected from Tables 4, 5 or 6. In some embodiments, the antibody epitope includes an allergen, or a fragment thereof, selected from Table 5. In some embodiments, the antibody epitope includes an infectious agent-derived biomolecule, or a fragment thereof, selected from Table 6.

In any embodiment, the signal-amplifying nanosensor may contain a plurality of antibody epitopes selected from Tables 4, 5 and/or 6, wherein the reading step d) includes obtaining a measure of the amount of a plurality of epitope-binding antibodies in the sample, and wherein the amount of the plurality of epitope-binding antibodies in the sample is diagnostic of a disease or condition.

In any embodiment, the sample may be an environmental sample, and wherein the analyte may be an environmental marker. In some embodiments, the environmental marker is selected from Table 8.

In any embodiment, the method may include receiving or providing a report that indicates the safety or harmfulness for a subject to be exposed to the environment from which the sample was obtained.

In any embodiment, the method may include sending data containing the measured amount of the environmental marker to a remote location and receiving a report that indicates the safety or harmfulness for a subject to be exposed to the environment from which the sample was obtained.

In any embodiment, the signal-amplifying nanosensor array may include a plurality of capture agents that each binds to an environmental marker selected from Table 8, and wherein the reading step d) may include obtaining a measure of the amount of the plurality of environmental markers in the sample.

In any embodiment, the sample may be a foodstuff sample, wherein the analyte may be a foodstuff marker, and wherein the amount of the foodstuff marker in the sample may correlate with safety of the foodstuff for consumption. In some embodiments, the foodstuff marker is selected from Table 9.

In any embodiment, the method may include receiving or providing a report that indicates the safety or harmfulness for a subject to consume the foodstuff from which the sample is obtained.

In any embodiment, the method may include sending data containing the measured amount of the foodstuff marker to a remote location and receiving a report that indicates the safety or harmfulness for a subject to consume the foodstuff from which the sample is obtained.

In any embodiment, the signal-amplifying nanosensor array may include a plurality of capture agents that each binds to a foodstuff marker selected from Table 9, wherein the obtaining may include obtaining a measure of the amount of the plurality of foodstuff markers in the sample, and wherein the amount of the plurality of foodstuff marker in the sample may correlate with safety of the foodstuff for consumption.

Also provided herein are kits that find use in practicing the present method.

### BRIEF DESCRIPTION OF THE FIGURES

The skilled artisan will understand that the drawings, described below, are for illustration purposes only. The drawings are not intended to limit the scope of the present teachings in any way.

FIG. 1 depicts a schematic representation of a method of measuring the amount of a biomarker in a sample using a signal-amplifying nanosensor and a mobile device, according to embodiments of the invention.

FIG. 2 depicts a signal enhancing detector that includes a microfluidic nanosensor, according to embodiments of the invention.

FIG. 3 is a collection of images schematically representing a signal-amplifying nanosensor and an amyloid beta immunoassay using the same, according to embodiments of the invention.

FIG. 4 is a collection of graphs showing immunoassay standard curves for different biomarkers on signal-amplifying nanosensor, according to embodiments of the invention.

FIG. 5 is a graph showing monitoring of salivary beta amyloid 1-42 levels in healthy human subjects using a signal-amplifying nanosensor, according to embodiments of the invention.

FIG. 6 is a collection of drawings and a graph showing a schematic of a signal-amplifying nanosensor device, an electron micrograph of the nanostructured surface and data showing enhancement of fluorescence compared to a glass surface.

FIG. 7 is a table of common biomarkers for brain function and damage.

FIG. 8 is a collection of images showing a schematic of a method of producing a signal-amplifying nanosensor biomarker testing device and a method of using the same.

FIG. 9 is a schematic representation of the smart phone-based personal health monitoring method, according to embodiments of the invention.

### DEFINITIONS

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid”, “nucleic acid molecule”, “nucleic acid sequence” and “oligonucleotide” are used interchangeably, and can also include plurals of each respectively depending on the context in which the terms are utilized. They refer to a polymeric form of nucleotides of any length, either deoxyribonucleotides (DNA) or ribonucleotides (RNA), or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA, ribozymes, small interfering RNA, (siRNA), microRNA (miRNA), small nuclear RNA (snRNA), cDNA, recombinant polynucleotides, branched polynucleotides, plasmids, vectors, isolated DNA (A, B and Z structures) of any sequence, PNA, locked nucleic acid (LNA), TNA (treose nucleic acid), isolated RNA of any sequence, nucleic acid probes, and primers. LNA, often referred to as inaccessible RNA, is a modified RNA nucleotide. The ribose moiety of an LNA nucleotide is modified with an extra bridge connecting the 2′ and 4′ carbons. The bridge “locks” the ribose in the 3′-endo structural conformation, which is often found in the A-form of DNA or RNA, which can significantly improve thermal stability.

A “capture agent” as used herein, refers to a binding member, e.g. nucleic acid molecule, polypeptide molecule, or any other molecule or compound, that can specifically bind to its binding partner, e.g., a second nucleic acid molecule containing nucleotide sequences complementary to a first nucleic acid molecule, an antibody that specifically recognizes an antigen, an antigen specifically recognized by an antibody, a nucleic acid aptamer that can specifically bind to a target molecule, etc. A capture agent may concentrate the target molecule from a heterogeneous mixture of different molecules by specifically binding to the target molecule. Binding may be non-covalent or covalent.

The affinity between a binding member and its binding partner to which it specifically binds when they are specifically bound to each other in a binding complex is characterized by a K_{D} (dissociation constant) of 10⁻⁵ M or less, 10⁻⁶ M or less, such as 10-7 M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less. “Affinity” refers to the strength of binding, increased binding affinity being correlated with a lower K_{D}.

The term “a secondary capture agent” which can also be referred to as a “detection agent” refers a group of biomolecules or chemical compounds that have highly specific affinity to the antigen. The secondary capture agent can be strongly linked to an optical detectable label, e.g., enzyme, fluorescence label, or can itself be detected by another detection agent that is linked to an optical detectable label through bioconjugation (Hermanson, “Bioconjugate Techniques” Academic Press, 2nd Ed., 2008).

By “antibody,” as used herein, is meant a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA antibody “isotypes” or “classes” respectively. Antibody herein is meant to include full length antibodies and antibody fragments, and may refer to a natural antibody from any organism, an engineered antibody, or an antibody generated recombinantly for experimental, therapeutic, or other purposes. The term “antibody” includes full length antibodies, and antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies.

The terms “antibody epitope,” “epitope,” “antigen” are used interchangeably herein to refer to a biomolecule that is bound by an antibody. Antibody epitopes can include proteins, carbohydrates, nucleic acids, hormones, receptors, tumor markers, and the like, and mixtures thereof. An antibody epitope can also be a group of antibody epitopes, such as a particular fraction of proteins eluted from a size exclusion chromatography column. Still further, an antibody epitope can also be identified as a designated clone from an expression library or a random epitope library.

An “allergen,” as used herein is a substance that elicits an allergic, inflammatory reaction in an individual when the individual is exposed to the substance, e.g., by skin contact, ingestion, inhalation, eye contact, etc. An allergen may include a group of substances that together elicit the allergic reaction. Allergens may be found in sources classified by the following groups: natural and artificial fibers (cotton, linen, wool, silk, teak, etc., wood, straw, and other dust); tree pollens (alder, birch, hazel, oak, poplar, palm, and others); weeds and flowers (ambrosia, artemisia, and others); grasses and corns (fescue, timothy grass, rye, wheat, corn, bluegrass, and others); drugs (antibiotics, antimicrobial drugs, analgetics and non-steroid anti-inflammatory drugs, anesthetics and muscle relaxants, hormones, and others); epidermal and animal allergens (epithelium of animals, feathers of birds, sera, and others); molds and yeasts (Penicillium notation, Cladosporium spp., Aspergillus fumigatus, Mucor racemosus, and others); insect venoms; preservatives (butylparaben, sorbic acid, benzoate, and others); semen (ejaculate); parasitic and mite allergens (ascarids, Dermatophagoides pteronyssinus, Dermatophagoides farinae, Euroglyphus maynei, and others); occupational and hobby allergens (coffee beans, formaldehyde, latex, chloramine, dyes, and others); food allergens (egg products, dairy products and cheeses, meat products, fish and seafood, soy products, mushrooms, flours and cereals, vegetables, melons and gourds, beans, herbs and spices, nuts, citrus and other fruits, berries, teas and herbs, nutritional supplements, and other products), etc.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these.

As is known to one skilled in the art, hybridization can be performed under conditions of various stringency. Suitable hybridization conditions are such that the recognition interaction between a capture sequence and a target nucleic acid is both sufficiently specific and sufficiently stable. Conditions that increase the stringency of a hybridization reaction are widely known and published in the art. See, for example, Green, et al., (2012), infra.

“Conditions suitable for binding,” as used herein with respect to binding of a capture agent to an analyte, e.g., a biomarker, a biomolecule, a synthetic organic compound, an inorganic compound, etc., refers to conditions that produce nucleic acid duplexes, protein/protein (e.g., antibody/antigen) complexes, protein/compound complexes, aptamer/target complexes that contain pairs of molecules that specifically bind to one another, while, at the same time, disfavor the formation of complexes between molecules that do not specifically bind to one another. Specific binding conditions are the summation or combination (totality) of both hybridization and wash conditions, and may include a wash and blocking steps, if necessary.

For nucleic acid hybridization, specific binding conditions can be achieved by incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C.

For binding of an antibody to an antigen, specific binding conditions can be achieved by blocking a substrate containing antibodies in blocking solution (e.g., PBS with 3% BSA or non-fat milk), followed by incubation with a sample containing analytes in diluted blocking buffer. After this incubation, the substrate is washed in washing solution (e.g. PBS+TWEEN 20) and incubated with a secondary capture antibody (detection antibody, which recognizes a second site in the antigen). The secondary capture antibody may conjugated with an optical detectable label, e.g., a fluorophore such as IRDye800CW, Alexa 790, Dylight 800. After another wash, the presence of the bound secondary capture antibody may be detected. One of skill in the art would be knowledgeable as to the parameters that can be modified to increase the signal detected and to reduce the background noise.

A “plurality” contains at least 2 members. In certain cases, a plurality may have at least 10, at least 100, at least 1000, at least 10,000, at least 100,000, at least 106, at least 107, at least 108 or at least 109 or more members.

The term “amplify” refers to an increase in the magnitude of a signal, e.g., at least a 10-fold increase, at least a 100-fold increase at least a 1,000-fold increase, at least a 10,000-fold increase, or at least a 100,000-fold increase in a signal.

A “microfluidic device” is a device that is configured to control and manipulate fluids geometrically constrained to a small scale (e.g., sub-millimeter).

A subject may be any human or non-human animal. A subject may be a person performing the instant method, a patient, a customer in a testing center, etc.

An “analyte,” as used herein is any substance that is suitable for testing in the present method.

As used herein, a “sample” refers to any bodily byproduct, such as bodily fluids, that has been derived from a subject. The sample may be obtained directly from the subject in the form of liquid, or may be derived from the subject by first placing the bodily byproduct in a solution, such as a buffer. Exemplary samples include, but are not limited to, saliva, serum, blood, sputum, urine, sweat, lacrima, semen, feces, biopsies, mucus, etc.

As used herein, a “diagnostic sample” refers to any biological sample that is a bodily byproduct, such as bodily fluids, that has been derived from a subject. The diagnostic sample may be obtained directly from the subject in the form of liquid, or may be derived from the subject by first placing the bodily byproduct in a solution, such as a buffer. Exemplary diagnostic samples include, but are not limited to, saliva, serum, blood, sputum, urine, sweat, lacrima, semen, feces, biopsies, mucus, etc.

As used herein, an “environmental sample” refers to any sample that is obtained from the environment. An environmental sample may include liquid samples from a river, lake, pond, ocean, glaciers, icebergs, rain, snow, sewage, reservoirs, tap water, drinking water, etc.; solid samples from soil, compost, sand, rocks, concrete, wood, brick, sewage, etc.; and gaseous samples from the air, underwater heat vents, industrial exhaust, vehicular exhaust, etc. Typically, samples that are not in liquid form are converted to liquid form before analyzing the sample with the present method.

As used herein, a “foodstuff sample” refers to any sample that is suitable for animal consumption, e.g., human consumption. A foodstuff sample may include raw ingredients, cooked food, plant and animal sources of food, preprocessed food as well as partially or fully processed food, etc. Typically, samples that are not in liquid form are converted to liquid form before analyzing the sample with the present method.

The term “diagnostic,” as used herein, refers to the use of a method or an analyte for identifying, predicting the outcome of and/or predicting treatment response of a disease or condition of interest. A diagnosis may include predicting the likelihood of or a predisposition to having a disease or condition, estimating the severity of a disease or condition, determining the risk of progression in a disease or condition, assessing the clinical response to a treatment, and/or predicting the response to treatment.

A “biomarker,” as used herein, is any molecule or compound that is found in a sample of interest and that is known to be diagnostic of or associated with the presence of or a predisposition to a disease or condition of interest in the subject from which the sample is derived. Biomarkers include, but are not limited to, polypeptides or a complex thereof (e.g., antigen, antibody), nucleic acids (e.g., DNA, miRNA, mRNA), drug metabolites, lipids, carbohydrates, hormones, vitamins, etc., that are known to be associated with a disease or condition of interest.

A “condition” as used herein with respect to diagnosing a health condition, refers to a physiological state of mind or body that is distinguishable from other physiological states. A health condition may not be diagnosed as a disease in some cases. Exemplary health conditions of interest include, but are not limited to, nutritional health; aging; exposure to environmental toxins, pesticides, herbicides, synthetic hormone analogs; pregnancy; menopause; andropause; sleep; stress; prediabetes; exercise; fatigue; chemical balance; etc.

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the present disclosure.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

It must be noted that as used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

One with skill in the art will appreciate that the present invention is not limited in its application to the details of construction, the arrangements of components, category selections, weightings, pre-determined signal limits, or the steps set forth in the description or drawings herein. The invention is capable of other embodiments and of being practiced or being carried out in many different ways.

The practice of various embodiments of the present disclosure employs, unless otherwise indicated, conventional techniques of immunology, biochemistry, chemistry, molecular biology, microbiology, cell biology, genomics and recombinant DNA, which are within the skill of the art. See Green and Sambrook, MOLECULAR CLONING: A LABORATORY MANUAL, 4^{th} edition (2012); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### DETAILED DESCRIPTION

Provided herein is an analyte measurement method that employs a signal-amplifying nanosensor, i.e., a method for measuring the amount of an analyte in a sample using a signal-amplifying nanosensor. In certain embodiments, the method includes the steps of a) obtaining a sample, b) applying the sample to a signal-amplifying nanosensor containing a capture agent that binds to an analyte of interest, under conditions suitable for binding of the analyte in a sample to the capture agent, c) washing the signal-amplifying nanosensor, and d) reading the signal-amplifying nanosensor, thereby obtaining a measurement of the amount of the analyte in the sample. Further aspects of the present method and the signal-amplifying nanosensor are now described in more detail.

### Methods

As summarized above, aspects of the present disclosure include an analyte measurement method that includes the steps of obtaining a sample and applying the sample to a signal-amplifying nanosensor. The signal-amplifying nanosensor includes a capture agent that specifically binds to an analyte of interest, e.g., an analyte listed in Tables 1, 2, 3, 7, 8, and 9, or includes an antibody epitope, e.g., an epitope derived from targets listed in Tables 4, 5 and 6, that binds specifically to an antibody analyte of interest. Binding of the analyte to the capture agent may form an analyte-capture agent complex that is immobilized on the signal-amplifying nanosensor. Once the capture agent binds to the analyte of interest to form a detectably labeled, analyte-capture agent complex, the amount of bound analyte may be measured by reading the signal-amplifying nanosensor. Thus, the amount of analyte in the sample may be inferred from the amount of labeled analyte measured from the signal-amplifying nanosensor. Structural and chemical details of the signal-amplifying nanosensor are described in a later section below.

In certain embodiments, an analyte in the sample that is captured by the signal-amplifying nanosensor is labeled with a detectable label that binds, directly or indirectly, to the captured analyte. An analyte in the sample may be labeled using any convenient method, as described further below, and in some cases is labeled before applying the sample to the signal-amplifying nanosensor and binding the labeled analyte to the capture agent, or is labeled after, or at the same time as binding of the analyte to the capture agent on the signal-amplifying nanosensor. In certain embodiments, the signal-amplifying nanosensor is washed as necessary, for example, to remove any unbound sample components, e.g, proteins, nucleic acids, compounds, etc., that are not of interest, or to remove unbound label, etc.

The sample may vary depending on the analyte of interest that is to be detected. In some cases, the sample is a liquid sample. In other instances, if the analyte of interest is present in a first sample that is in solid or gaseous form, the first sample may be processed to provide the analyte of interest in a second sample that is in liquid form, e.g., by dissolving, comminuting and/or suspending the first sample in a suitable liquid, e.g., water, buffer, organic solvent, etc.

Any volume of sample may be applied to the signal-amplifying nanosensor.

Examples of volumes may include, but are not limited to, about 10 mL or less, 5 mL or less, 3 mL or less, 1 microliter (μL, also “uL” herein) or less, 500 μL, or less, 300 μL, or less, 250 μL, or less, 200 μL, or less, 170 μL, or less, 150 μL, or less, 125 μL, or less, 100 L, or less, 75 μL, or less, 50 μL, or less, 25 μL, or less, 20 μL, or less, 15 μL, or less, 10 L, or less, 5 μL, or less, 3 μL, or less, 1 μL, or less. The amount of sample may be about a drop of a sample. The amount of sample may be the amount collected from a pricked finger or fingerstick. The amount of sample may be the amount collected from a microneedle or a venous draw.

A sample may be used without further processing after obtaining it from the source, or may be processed, e.g., to enrich for an analyte of interest, remove large particulate matter, dissolve or resuspend a solid sample, etc.

Any suitable method of applying a sample to the signal-amplifying nanosensor may be employed. Suitable methods may include using a pipet, dropper, syringe, etc. In certain embodiments, when the signal-amplifying nanosensor is located on a support in a dipstick format, as described below, the sample may be applied to the signal-amplifying nanosensor by dipping a sample-receiving area of the dipstick into the sample.

A sample may be collected at one time, or at a plurality of times. Samples collected over time may be aggregated and/or processed (by applying to a signal-amplifying nanosensor and obtaining a measurement of the amount of analyte in the sample, as described herein) individually. In some instances, measurements obtained over time may be aggregated and may be useful for longitudinal analysis over time to facilitate screening, diagnosis, treatment, and/or disease prevention.

Washing the signal-amplifying nanosensor to remove unbound sample components may be done in any convenient manner, as described above. In certain embodiments, the surface of the signal-amplifying nanosensor is washed using binding buffer to remove unbound sample components.

Detectable labeling of the analyte may be done by any convenient method. The analyte may be labeled directly or indirectly. In direct labeling, the analyte in the sample is labeled before the sample is applied to the signal-amplifying nanosensor. In indirect labeling, an unlabeled analyte in a sample is labeled after the sample is applied to the signal-amplifying nanosensor to capture the unlabeled analyte, as described below.

Labeling the analyte may include using, for example, a labeling agent, such as an analyte specific binding member that includes a detectable label. Detectable labels include, but are not limited to, fluorescent labels, colorimetric labels, chemiluminescent labels, enzyme-linked reagents, multicolor reagents, avidin-streptavidin associated detection reagents, and the like. In certain embodiments, the detectable label is a fluorescent label. Fluorescent labels are labeling moieties that are detectable by a fluorescence detector. For example, binding of a fluorescent label to an analyte of interest may allow the analyte of interest to be detected by a fluorescence detector. Examples of fluorescent labels include, but are not limited to, fluorescent molecules that fluoresce upon contact with a reagent, fluorescent molecules that fluoresce when irradiated with electromagnetic radiation (e.g., UV, visible light, x-rays, etc.), and the like.

Suitable fluorescent molecules (fluorophores) include, but are not limited to, IRDye800CW, Alexa 790, Dylight 800, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethyl rhodamine methyl ester), TMRE (tetramethyl rhodamine ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives, such as acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120),7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM),5-(4,6-dichlorotriazin-2-yl)amino-fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl) aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, fluorescent europium and terbium complexes; combinations thereof, and the like. Suitable fluorescent proteins and chromogenic proteins include, but are not limited to, a green fluorescent protein (GFP), including, but not limited to, a GFP derived from Aequoria victoria or a derivative thereof, e.g., a “humanized” derivative such as Enhanced GFP; a GFP from another species such as Renilla remformis, Renilla mulleri, or Ptilosarcus guernyi; “humanized” recombinant GFP (hrGFP); any of a variety of fluorescent and colored proteins from Anthozoan species; combinations thereof; and the like.

In certain embodiments, the labeling agent is configured to bind specifically to the analyte of interest. In certain embodiments, a labeling agent may be present in the signal-amplifying nanosensor before the sample is applied to the signal-amplifying nanosensor.

In other embodiments, the labeling agent may be applied to the signal-amplifying nanosensor after the sample is applied to the signal-amplifying nanosensor. In certain embodiments, after the sample is applied to the signal-amplifying nanosensor, the signal-amplifying nanosensor may be washed to remove any unbound components, e.g. un bound analyte and other non-analyte coponents in the sample, and the labeling agent may be applied to the signal-amplifying nanosensor after the washing to label the bound analyte. In some embodiments, the signal-amplifying nanosensor may be washed after the labeling agent is bound to the analyte-capture agent complex to remove from the signal-amplifying nanosensor any excess labeling agent that is not bound to an analyte-capture agent complex.

In certain embodiments, the analyte is labeled after the analyte is bound to the signal-amplifying nanosensor, e.g., using a labeled binding agent that can bind to the analyte simultaneously as the capture agent to which the analyte is bound in the signal-amplifying nanosensor, i.e., in a sandwich-type assay. In some embodiments, a nucleic acid analyte may be captured on the signal-amplifying nanosensor, and a labeled nucleic acid that can hybridize to the analyte simultaneously as the capture agent to which the nucleic acid analyte is bound in the signal-amplifying nanosensor.

In certain aspects, a signal-amplifying nanosensor enhances the light signal, e.g., fluorescence or luminescence, that is produced by the detectable label bound directly or indirectly to an analyte, which is in turn bound to the signal-amplifying nanosensor. In certain embodiments, the signal is enhanced by a physical process of signal amplification. In some embodiments, the light signal is enhanced by a nanoplasmonic effect (e.g., surface-enhanced Raman scattering). Examples of signal enhancement by nanoplasmonic effects is described, e.g., in Li et al, Optics Express 2011 19: 3925-3936 and WO2012/024006, which are incorporated herein by reference. In certain embodiments, signal enhancement is achieved without the use of biological/chemical amplification of the signal. Biological/chemical amplification of the signal may include enzymatic amplification of the signal (e.g., used in enzyme-linked immunosorbent assays (ELISAs)) and polymerase chain reaction (PCR) amplification of the signal. In other embodiments, the signal enhancement may be achieved by a physical process and biological/chemical amplification.

In certain embodiments, the signal-amplifying nanosensor is configured to enhance the signal from a detectable label that is proximal to the surface of the signal-amplifying nanosensor by 10³ fold or more, for example, 10⁴ fold or more, 10⁵ fold or more, 10⁶ fold or more, 10⁷ fold or more, including 10⁸ fold or more, where the signal may be enhanced by a range of 10³ to 10⁹ fold, for example, 10⁴ to 10⁸ fold, or 10⁵ to 10⁷ fold, compared to a detectable label that is not proximal to the surface of the signal-amplifying nanosensor, i.e., compared to a detectable label bound to an analyte on a conventional ELISA plate, on a conventional nucleic acid microarray, suspended in solution, etc. In certain embodiments, the signal-amplifying nanosensor is configured to enhance the signal from a detectable label that is proximal to the surface of the signal-amplifying nanosensor by 10³ fold or more, for example, 10⁴ fold or more, 10⁵ fold or more, 10⁶ fold or more, 10⁷ fold or more, including 10⁸ fold or more, where the signal may be enhanced by a range of 10³ to 10⁹ fold, for example, 10⁴ to 10⁸ fold, or 10⁵ to 10⁷ fold, compared to an analyte detecting array that is not configured to enhance the signal using a physical amplification process, as described above. In certain embodiments, the signal-amplifying nanosensor is configured to have a detection sensitivity of 0.1 nM or less, such as 10 pM or less, or 1 pM or less, or 100 fM or less, such as 10 fM or less, including 1 fM or less, or 0.5 fM or less, or 100 aM or less, or 50 aM or less, or 20 aM or less. In certain embodiments, the signal-amplifying nanosensor is configured to have a detection sensitivity in the range of 10 aM to 0.1 nM, such as 20 aM to 10 pM, 50 aM to 1 pM, including 100 aM to 100 fM. In some instances, the signal-amplifying nanosensor is configured to be able to detect analytes at a concentration of 1 ng/mL or less, such as 100 pg/mL or less, including 10 pg/mL or less, 1 pg/mL or less, 100 fg/mL or less, 10 fg/mL or less, or 5 fg/mL or less. In some instances, the signal-amplifying nanosensor is configured to be able to detect analytes at a concentration in the range of 1 fg/mL to 1 ng/mL, such as 5 fg/mL to 100 pg/mL, including 10 fg/mL to 10 pg/mL. In certain embodiments, the signal-amplifying nanosensor is configured to have a dynamic range of 5 orders of magnitude or more, such as 6 orders of magnitude or more, including 7 orders of magnitude or more.

In certain instances, the period of time from applying the sample to the signal-amplifying nanosensor to reading the signal-amplifying nanosensor may range from 1 second to 30 minutes, such as 10 seconds to 20 minutes, 30 seconds to 10 minutes, including 1 minute to 5 minutes. In some instances, the period of time from applying the sample to the signal enhancing detector to generating an output that can be received by the device may be 1 hour or less, 30 minutes or less, 15 minutes or less, 10 minutes or less, 5 minutes or less, 3 minutes or less, 1 minute or less, 50 seconds or less, 40 seconds or less, 30 seconds or less, 20 seconds or less, 10 seconds or less, 5 seconds or less, 2 seconds or less, 1 second or less, or even shorter. In some instances, the period of time from applying the sample to the signal enhancing detector to generating an output that can be received by the device may be 100 milliseconds or more, including 200 milliseconds or more, such as 500 milliseconds or more, 1 second or more, 10 seconds or more, 30 seconds or more, 1 minute or more, 5 minutes or more, or longer.

Any suitable method may be used to read the signal-amplifying nanosensor to obtain a measurement of the amount of analyte in the sample. In some embodiments, reading the signal-amplifying nanosensor includes obtaining an electromagnetic signal from the detectable label bound to the analyte in the signal-amplifying nanosensor. In certain embodiments the electromagnetic signal is a light signal. The light signal obtained may include the intensity of light, the wavelength of light, the location of the source of light, and the like. In particular embodiments, the light signal produced by the label has a wavelength that is in the range of 300 nm to 900 nm. In certain embodiments, the light signal is read in the form of a visual image of the signal-amplifying nanosensor.

In certain embodiments, reading the signal-amplifying nanosensor includes providing a source of electromagnetic radiation, e.g., light source, as an excitation source for the detectable label bound to the biomarker in the signal-amplifying nanosensor. The light source may be any suitable light source to excite the detectable label. Exemplary light sources include, but are not limited to, sun light, ambient light, UV lamps, fluorescent lamps, light-emitting diodes (LEDs), photodiodes, incandescent lamps, halogen lamps, and the like.

Reading the signal-amplifying nanosensor may be achieved by any suitable method to measure the amount of analyte that is present in the sample and bound to the signal-amplifying nanosensor. In certain embodiments, the signal-amplifying nanosensor is read with a device configured to acquire the light signal from the detectable label bound to the analyte in the signal-amplifying nanosensor. In some cases, the device is a handheld device, such as a mobile phone or a smart phone. Any suitable handheld device configured to read the signal-amplifying nanosensor may be used in the present method. Devices configured to read the signal-amplifying nanosensor are described in, e.g., U.S. Provisional Application Ser. No. 62/066,777, filed on Oct. 21, 2014, which is incorporated herein by reference.

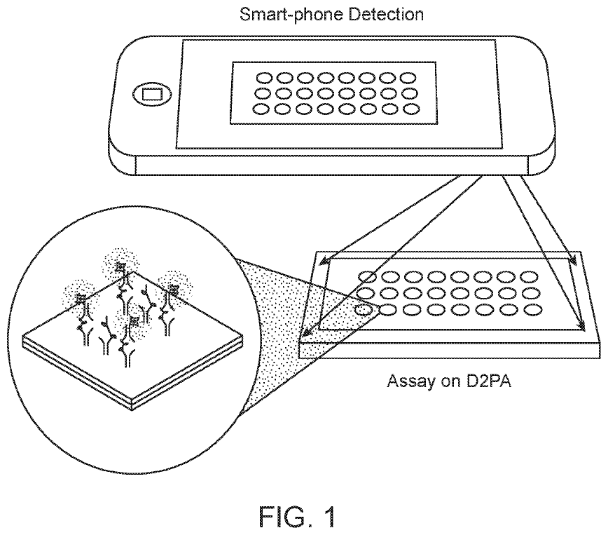

In some embodiments, the device includes an optical recording apparatus that is configured to acquire a light signal from the signal-amplifying nanosensor, e.g., acquire an image of the signal-amplifying nanosensor (FIG. 1). In certain instances, the optical recording apparatus is a camera, such as a digital camera. The term “digital camera” denotes any camera that includes as its main component an image-taking apparatus provided with an image-taking lens system for forming an optical image, an image sensor for converting the optical image into an electrical signal, and other components, examples of such cameras including digital still cameras, digital movie cameras, and Web cameras (i.e., cameras that are connected, either publicly or privately, to an apparatus connected to a network to permit exchange of images, including both those connected directly to a network and those connected to a network by way of an apparatus, such as a personal computer, having an information processing capability). In one example, reading the signal-amplifying nanosensor may include video imaging that may capture changes over time. For example, a video may be acquired to provide evaluation on dynamic changes in the sample applied to the signal-amplifying nanosensor.

In certain embodiments, the optical recording apparatus has a sensitivity that is lower than the sensitivity of a high-sensitivity optical recording apparatus used in research/clinical laboratory settings. In certain cases, the optical recording apparatus used in the subject method has a sensitivity that is lower by 10 times or more, such as 100 times or more, including 200 times or more, 500 times or more, or 1,000 times or more than the sensitivity of a high-sensitivity optical recording apparatus used in research/clinical laboratory settings.

In certain embodiments, the device may have a video display. Video displays may include components upon which a display page may be displayed in a manner perceptible to a user, such as, for example, a computer monitor, cathode ray tube, liquid crystal display, light emitting diode display, touchpad or touchscreen display, and/or other means known in the art for emitting a visually perceptible output. In certain embodiments, the device is equipped with a touch screen for displaying information, such as the image acquired from the detector and/or a report generated from the processed data, and allowing information to be entered by the subject.

In certain embodiments, the subject device is configured to process data derived from reading the signal-amplifying nanosensor. The device may be configured in any suitable way to process the data for use in the subject methods. In certain embodiments, the device has a memory location to store the data and/or store instructions for processing the data and/or store a database. The data may be stored in memory in any suitable format.

In certain embodiments, the device has a processor to process the data. In certain embodiments, the instructions for processing the data may be stored in the processor, or may be stored in a separate memory location. In some embodiments, the device may contain a software to implement the processing.

In certain embodiments, a device configured to process data acquired from the signal-amplifying nanosensor device contains software implemented methods to perform the processing. Software implemented methods may include one or more of: image acquisition algorithms; image processing algorithms; user interface methods that facilitate interaction between user and computational device and serves as means for data collection, transmission and analysis, communication protocols; and data processing algorithms. In certain embodiments, image processing algorithms include one or more of: a particle count, a LUT (look up table) filter, a particle filter, a pattern recognition, a morphological determination, a histogram, a line profile, a topographical representation, a binary conversion, or a color matching profile.

In certain embodiments, the device is configured to display information on a video display or touchscreen display when a display page is interpreted by software residing in memory of the device. The display pages described herein may be created using any suitable software language such as, for example, the hypertext markup language (“HTML”), the dynamic hypertext markup language (“DHTML”), the extensible hypertext markup language (“XHTML”), the extensible markup language (“XML”), or another software language that may be used to create a computer file displayable on a video or other display in a manner perceivable by a user. Any computer readable media with logic, code, data, instructions, may be used to implement any software or steps or methodology. Where a network comprises the Internet, a display page may comprise a webpage of a suitable type.

A display page according to the invention may include embedded functions comprising software programs stored on a memory device, such as, for example, VBScript routines, JScript routines, JavaScript routines, Java applets, ActiveX components, ASP.NET, AJAX, Flash applets, Silverlight applets, or AIR routines.

A display page may comprise well known features of graphical user interface technology, such as, for example, frames, windows, scroll bars, buttons, icons, and hyperlinks, and well known features such as a “point and click” interface or a touchscreen interface. Pointing to and clicking on a graphical user interface button, icon, menu option, or hyperlink also is known as “selecting” the button, option, or hyperlink. A display page according to the invention also may incorporate multimedia features, multi-touch, pixel sense, IR LED based surfaces, vision-based interactions with or without cameras.

A user interface may be displayed on a video display and/or display page. The user interface may display a report generated based on analyzed data relating to the sample, as described further below.

The processor may be configured to process the data in any suitable way for use in the subject methods. The data is processed, for example, into binned data, transformed data (e.g., time domain data transformed by Fourier Transform to frequency domain), or may be combined with other data. The processing may put the data into a desired form, and may involve modifying the format of data. Processing may include detection of a signal from a sample, correcting raw data based on mathematical manipulation or correction and/or calibrations specific for the device or reagents used to examine the sample; calculation of a value, e.g., a concentration value, comparison (e.g., with a baseline, threshold, standard curve, historical data, or data from other sensors), a determination of whether or not a test is accurate, highlighting values or results that are outliers or may be a cause for concern (e.g., above or below a normal or acceptable range, or indicative of an abnormal condition), or combinations of results which, together, may indicate the presence of an abnormal condition, curve-fitting, use of data as the basis of mathematical or other analytical reasoning (including deductive, inductive, Bayesian, or other reasoning), and other suitable forms of processing. In certain embodiments, processing may involve comparing the processed data with a database stored in the device to retrieve instructions for a course of action to be performed by the subject.

In certain embodiments, the device may be configured to process the input data by comparing the input data with a database stored in a memory to retrieve instructions for a course of action to be performed by the subject. In some embodiments, the database may contain stored information that includes a threshold value for the analyte of interest. The threshold value may be useful for determining the presence or concentration of the one or more analytes. The threshold value may be useful for detecting situations where an alert may be useful. The data storage unit may include records or other information that may be useful for generating a report relating to the sample.

In certain embodiments, the device may be configured to receive data that is derived from the signal-amplifying nanosensor. Thus in certain cases, the device may be configured to receive data that is not related to the sample provided by the subject but may still be relevant to the diagnosis. Such data include, but are not limited to the age, sex, height, weight, individual and/or family medical history, etc. In certain embodiments, the device is configured to process data derived from or independently from a sample applied to the signal-amplifying nanosensor.

In certain embodiments the device may be configured to communicate over a network such as a local area network (LAN), wide area network (WAN) such as the Internet, personal area network, a telecommunications network such as a telephone network, cell phone network, mobile network, a wireless network, a data-providing network, or any other type of network. In certain embodiments the device may be configured to utilize wireless technology, such as Bluetooth or RTM technology. In some embodiments, the device may be configured to utilize various communication methods, such as a dial-up wired connection with a modem, a direct link such as TI, integrated services digital network (ISDN), or cable line. In some embodiments, a wireless connection may be using exemplary wireless networks such as cellular, satellite, or pager networks, general packet radio service (GPRS), or a local data transport system such as Ethernet or token ring over a LAN. In some embodiments, the device may communicate wirelessly using infrared communication components.

In certain embodiments, the device is configured to receive a computer file, which can be stored in memory, transmitted from a server over a network. The device may receive tangible computer readable media, which may contain instructions, logic, data, or code that may be stored in persistent or temporary memory of the device, or may affect or initiate action by the device. One or more devices may communicate computer files or links that may provide access to other computer files.

In some embodiments, the device is a personal computer, server, laptop computer, mobile device, tablet, mobile phone, cell phone, satellite phone, smartphone (e.g., iPhone, Android, Blackberry, Palm, Symbian, Windows), personal digital assistant, Bluetooth device, pager, land-line phone, or other network device. Such devices may be communication-enabled devices. The term “mobile phone” as used herein refers to a telephone handset that can operate on a cellular network, a Voice-Over IP (VoIP) network such as Session Initiated Protocol (SIP), or a Wireless Local Area Network (WLAN) using an 802.11x protocol, or any combination thereof. In certain embodiments, the device can be hand-held and compact so that it can fit into a consumer's wallet and/or pocket (e.g., pocket-sized).

In certain embodiments, the signal-amplifying nanosensor is integrated into a solid support or platform. In some embodiments, the signal-amplifying nanosensor is integrated into a nanosensor device that includes a platform or support. In certain embodiments, the nanosensor device is a microfluidic platform or device. The microfluidic device may be configured to have different areas for receiving a sample, detecting analytes in the sample with a signal-amplifying nanosensor, collecting waste material in a reservoir, etc. Thus, in certain embodiments, the microfluidic channel platform may include fluid handling components to direct a sample applied to a sample receiving area of the microfluidic device to a signal-amplifying nanosensor configured to detect an analyte, as described above. The fluid handling components may be configured to direct one or more fluids through the microfluidic device. In some instances, the fluid handling components are configured to direct fluids, such as, but not limited to, a sample solution, buffers and the like. Liquid handling components may include, but are not limited to, passive pumps and microfluidic channels. In some cases, the passive pumps are configured for capillary action-driven microfluidic handling and routing of fluids through the microfluidic device disclosed herein. In certain instances, the microfluidic fluid handling components are configured to deliver small volumes of fluid, such as 1 mL or less, such as 500 μL or less, including 100 μL or less, for example 50 μL or less, or 25 L or less, or 10 μL or less, or 5 μL or less, or 1 μL or less. Thus, in certain embodiments, no external source of power is required to operate the microfluidic device and perform the present method.

In certain embodiments, the microfluidic device has dimensions in the range of 5 mm×5 mm to 100 mm×100 mm, including dimensions of 50 mm×50 mm or less, for instance 25 mm×25 mm or less, or 10 mm×10 mm or less. In certain embodiments, the microfluidic device has a thickness in the range of 5 mm to 0.1 mm, such as 3 mm to 0.2 mm, including 2 mm to 0.3 mm, or 1 mm to 0.4 mm.

In certain embodiments, the signal-amplifying nanosensor is integrated on a dipstick structure or a lateral flow format, examples of which is described in, e.g., U.S. Pat. No. 6,660,534, incorporated herein by reference.

In certain embodiments, the signal-amplifying nanosensor is disposed within a container, e.g., a well of a multi-well plate. The signal-amplifying nanosensor also can be integrated into the bottom or the wall of a well of a multi-well plate.

In some embodiments, a support containing a signal-amplifying nanosensor, such as a microfluidic device or multi-well plate, may have an identifier for the signal-amplifying nanosensor that is contained in the support. An identifier may be a physical object formed on the support, such as a microfluidic device. For example, the identifier may be read by a handheld device, such as a mobile phone or a smart phone, as described above. In some embodiments, a camera may capture an image of the identifier and the image may be analyzed to identify the signal-amplifying nanosensor contained in the microfluidic device. In one example, the identifier may be a barcode. A barcode may be a 1D or 2D barcode. In some embodiments, the identifier may emit one or more signal that may identify the signal enhancing detector. For example, the identifier may provide an infrared, ultrasonic, optical, audio, electrical, or other signal that may indicate the identity of the signal-amplifying nanosensor. The identifier may utilize a radiofrequency identification (RFID) tag.

The identifier may contain information that allows determination of the specific type of signal-amplifying nanosensor present in a microfluidic device or multi-well plate. In certain embodiments, the identifier provides a key to a database that associates each identifier key to information specific to the type of signal-amplifying nanosensor present in a microfluidic device or multi-well plate. The information specific to the type of signal-amplifying nanosensor may include, but are not limited to, the identity of the analytes which the signal-amplifying nanosensor configured to detect, the coordinates of the position where a specific analyte may bind on the signal-amplifying nanosensor, the sensitivity of detection for each analyte, etc. The database may contain other information relevant to a specific signal-amplifying nanosensor, including an expiration date, lot number, etc. The database may be present on a handheld device, provided on a computer-readable medium, or may be on a remote server accessible by a handheld device.

Further aspects of the subject method include providing or receiving a report that indicates the measured amount of the analyte and other information pertinent to the source from which the analyte was obtained, e.g., diagnoses or health status for a diagnostic sample, exposure risk for an environmental sample, health risk for a foodstuff sample, etc. The report may be provided or received in any convenient form, including, but not limited to, by viewing the report displayed on a screen on the device, by viewing an electronic mail or text message sent to the subject, by listening to an audio message generated by the device, by sensing a vibration generated by the device, etc.

The report may contain any suitable information that is pertinent to the source from which the analyte was obtained. In some instances, the report may include: light data, including light intensity, wavelength, polarization, and other data regarding light, e.g., output from optical detectors such as photomultiplier tubes, photodiodes, charge-coupled devices, luminometers, spectrophotometers, cameras, and other light sensing components and devices, including absorbance data, transmittance data, turbidity data, luminosity data, wavelength data (including intensity at one, two, or more wavelengths or across a range of wavelengths), reflectance data, refractance data, birefringence data, polarization, and other light data; image data, e.g., data from digital cameras; the identifier information associated with the signal-amplifying nanosensor used to acquire the data; the processed data, as described above, etc. The report may represent qualitative or quantitative aspects of the sample.

In certain aspects, the report may indicate to the subject the presence or absence of an analyte, the concentration of an analyte, the presence or absence of a secondary condition known to be correlated with the presence or level of the analyte, the probability or likelihood of a secondary condition known to be correlated with the presence or level of the analyte, the likelihood of developing a secondary condition known to be correlated with the presence or level of the analyte, the change in likelihood of developing a secondary condition known to be correlated with the presence or level of the analyte, the progression of a secondary condition known to be correlated with the presence or level of the analyte, etc. The secondary condition known to be correlated with the presence or level of the analyte may include a disease or health condition for a diagnostic sample, a toxic or otherwise harmful environment for an environmental sample, spoiled or tainted food for a foodstuff sample, etc. In certain embodiments, the report contains instructions urging or recommending the user to take action, such as seek medical help, take medication, stop an activity, start an activity, etc. The report may include an alert. One example of an alert may be if an error is detected on the device, or if an analyte concentration exceeds a predetermined threshold. The content of the report may be represented in any suitable form, including text, graphs, graphics, animation, color, sound, voice, and vibration.

In certain embodiment, the report provides an action advice to the user of the subject device, e.g., a mobile phone. The advices will be given according to the test data by the devices (e.g. detectors plus mobile phone) together with one or several data sets, including but not limited to, the date preloaded on the mobile devices, data on a storage device that can be accessed, where the storage device can be locally available or remotely accessible.

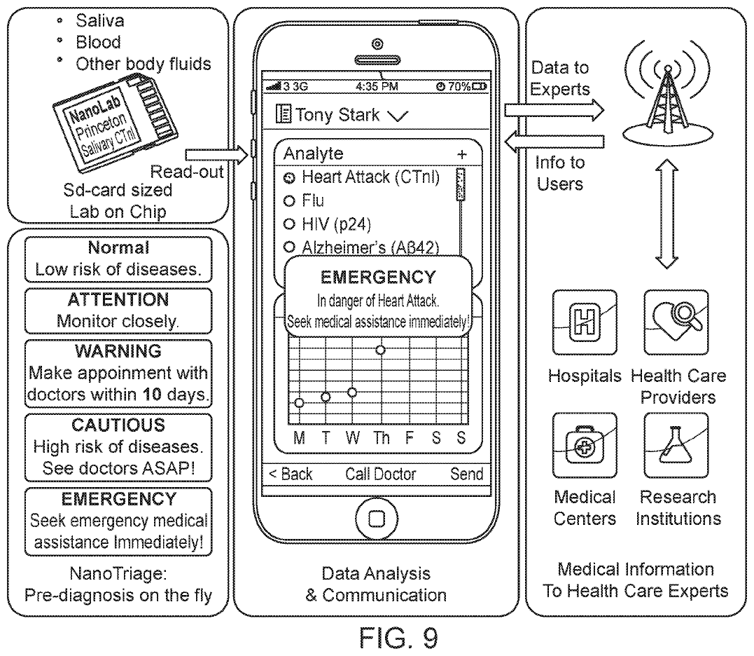

In certain embodiments, each of the advices above has its own color in scheme in the mobile phone displays. One example is given in FIG. 9.

In certain embodiments, the present method includes sending data containing the measured amount of the analyte to a remote location and receiving an analysis, e.g., diagnosis, safety information, etc., from the remote location. Transmitting the data to a remote location may be achieved by any convenient method, as described above. Such transmissions may be via electronic signals, radiofrequency signals, optical signals, cellular signals, or any other type of signals that may be transmitted via a wired or wireless connection. Any transmission of data or description of electronic data or transmission described elsewhere herein may occur via electronic signals, radiofrequency signals, optical signals, cellular signals, or any other type of signals that may be transmitted via a wired or wireless connection. The transmitted data may include the data derived from the signal-amplifying nanosensor and/or the processed data and/or the generated report. The transmitted data may also include data that was not acquired from the signal-amplifying nanosensor, i.e., data that does not directly represent an aspect of the sample obtained from the subject, but does represent other aspects of the subject from which the sample was obtained, as described above.

Further aspects of the present disclosure include a signal-amplifying nanosensor that includes a plurality of capture agents that each binds to a plurality of analytes in a sample, i.e., a multiplexed signal-amplifying nanosensor. In such instances, the signal-amplifying nanosensor containing a plurality of capture agents may be configured to detect different types of analytes (protein, nucleic acids, antibodies, etc.). The different analytes may be distinguishable from each other on the array based on the location within the array, the emission wavelength of the detectable label that binds to the different analytes, or a combination of the above.

In certain embodiments, the present method includes applying a control sample to a control signal-amplifying nanosensor containing a capture agent that binds to the analyte, wherein the control sample contains a known detectable amount of the analyte, and reading the control signal-amplifying nanosensor, thereby obtaining a control measurement for the known detectable amount of the analyte in a sample. In certain embodiments, when the signal-amplifying nanosensor is present in a microfluidic device, the control signal-amplifying nanosensor may be present in the same device as the signal-amplifying nanosensor to which the test sample is applied. In certain embodiments, the control measurement obtained from the control sample may be used to obtain the absolute amount of the analyte in a test sample. In certain embodiments, the control measurement obtained from the control sample may be used to obtain a standardized relative amount of the analyte in a test sample.

### Nanosensors Comprising a Signal Amplification Layer (SAL)

A signal amplification layer generally comprises nanoscale metal-dielectric/semiconductor-metal structures, which amplifies local surface electric field and gradient and light signals. The amplification are the high at the location where there are the sharp (i.e. large curvature) edges of a metal structure and the between a small gaps of the two metal structures. The highest enhancement regions are those having both the sharp edges and the small gaps. Furthermore, the preferred dimensions for all metallic and non-metallic micro/nanostructures should be less than the wavelength of the light the signal amplification layer amplifies (i.e. subwavelength).

A signal amplification layer layer may have as many the metallic sharp edges and the small gaps as possible. This requires having dense of metallic nanostructures with small gaps apart. The invention includes several different signal amplification layer structures. Furthermore, the signal amplification layer itself can be further improved by a process that can further cover the portions of the metallic materials that do not have sharp edges and small gaps, as described in U.S. provisional application Ser. No. 61/801,424, filed on Mar. 15, 2013, and copending PCT application entitled “Methods for enhancing assay sensing properties by selectively masking local surfaces”, filed on Mar. 15, 2014, which are incorporated by reference.

The light amplification comes from one or several following factors: the nanosensor can (a) absorb light excitation effectively (e.g. the light at a wavelength that excites fluorescent moieties), (b) focus the absorbed light into certain locations, (c) place the analytes into the regions where most of light are focused, and (d) radiate efficiently the light generated by analytes from the locations where the analytes immobilized.

A signal amplifying nanosensor may comprise: (a) a substrate; (b) a signal amplification layer (SAL) on top of the substrate, (c) an optional molecular adhesion layer on the surface of the signal amplification layer, (d) a capture agent that specifically binds to the analyte, wherein the nanosensor amplifies a light signal from an analyte, when the analyte is bound to the capture agent. The signal amplification layer, comprising metallic and non-metallic micro/nanostructures, amplifies the sensing signal of the analytes captured by the capture agent, without an amplification of the number of molecules. Furthermore, such amplification is most effect within the very small depth (˜100 nm) from the SAL surface.

In any embodiment, a signal-amplifying nanosensor may comprise: (i) a substrate; (ii) a signal amplification layer comprising: a substantially continuous metallic backplane on the substrate; one or a plurality of pillars extending from the metallic backplane or from the substrate through holes in the backplane; and a metallic disk on top of the pillar, wherein at least one portion of the edge of the disk is separated from the metallic backplane; and (iii) a capture agent that specifically binds to an analyte in the sample, wherein the capture agent is linked to the surface of the signal amplification layer and said nanosensor amplifies a light signal from labeled analytes that are bound to the signal amplification layer via the capture The sensor amplifies a light signal that is proximal to the surface of the sensor. The sensor enhances local electric field and local electric field gradient in regions that is proximal to the surface of the sensor. The light signal includes light scattering, light diffraction, light absorption, nonlinear light generation and absorption, Raman scattering, chromaticity, luminescence that includes fluorescence, electroluminescence, chemiluminescence, and electrochemiluminescence, agent, under conditions suitable for binding of the analyte in a sample to the capture agent.

### Exemplary Embodiment for SAL Structures-1: Disk on Pillar (DoP)

Certain embodiments of the nanosensor, termed “disk on pillars” comprise: (a) a substrate; (b) a signal amplification layer comprising: (i) a substantially continuous metallic backplane on the substrate, (ii) one or a plurality of pillars extending from the metallic backplane or from the substrate through holes in the backplane, and (iii) a metallic disk on top of the pillar, wherein at least one portion of the edge of the disk has a small separation from one portion of the metallic backplane; (c) a capture agent that specifically binds to the analyte, wherein the capture agent is linked to the surface of the signal amplification layer; wherein the nanosensor amplifies a light signal from an analyte, when the analyte is bound to the capture agent.

When the pillars extend from the metallic backplane, the backplane has a sheet of film that goes under the pillar. When or from the substrate through holes in the backplane, the metallic backplane is near the foot of the pillar covering a substantial portion of the substrate surface. In some case, an nanosensor can by both types. The discs can have a lateral dimension either larger (preferred) or smaller or the same as the pillars. The advantages of former is the high signal amplification regions of the nanosensor are accessible to the analytes to be detected. The structure with disk lateral dimension larger than that of the pillar offers similar advantage, and hence preferred. In cases, additional etching in the fabrication to further reduce the pillar size while keeping the metallic disk size fixed. Furthermore, in certain embodiments, nanodots can be added to the outer surface of sidewall of the pillars.

The dimensions for metallic disks, the pillars, and the separations may be less than the wavelength of the light the signal amplification layer amplifies (i.e. subwavelength). For examples, for enhancing light of a wavelength of 400 nm to 1,000 nm (visible to near-infra-red), the separation should be 0.2 nm to 50 nm, preferably 0.2 to 25 nm, the average disc's lateral dimension is from 20 nm to 250 nm, and the disk thickness is from 5 nm to 60 nm, depending upon the light wavelength used in sensing.

### Exemplary Embodiment for SAL Structure-2: Random Metallic Nano-Islands with Metallic Backplane

In some embodiments, the metallic disc can be random metallic nano-islands. Such structure has a low cost advantage in certain situations. Such structure is termed “plasmonic cavity by metallic-island-sheet and metallic-backplane” (PCMM). The PCC comprises random metallic nanoislands located on top of a continuous dielectric film (instead of pillars) on top of a sheet of metal film.

### Exemplary Embodiment for SAL Structure-3: D2PA

A D2PA plate is a plate with a surface structure, termed “disk-coupled dots-on-pillar antenna array”, (D2PA), comprising: (a) substrate; and (b) a D2PA structure, on the surface of the substrate, comprising one or a plurality of pillars extending from a surface of the substrate, wherein at least one of the pillars comprises a pillar body, metallic disc on top of the pillar, metallic backplane at the foot of the pillar, the metallic backplane covering a substantial portion of the substrate surface near the foot of the pillar; metallic dot structure disposed on sidewall of the pillar. The D2PA amplifies a light signal that is proximal to the surface of the D2PA. The D2PA enhances local electric field and local electric field gradient in regions that is proximal to the surface of the D2PA.

Further description of DoP, random metallic nano-islands with a metallic backplane and D2PA sensors can be found in WO2014197097, which is incorporated by reference herein.

In some embodiments, different capture agents are attached to the nanosensor surface with each capture agent coated on a different location of the surface, e.g., in the form of an array, hence providing multiplexing in detections of different analysts, since each location is specific for capturing a specific kind of analyte.

In some embodiments, the nanosensor may be implemented in a multi-well format, e.g., a 24-well, a 96-well or 384 well format, where each well of a multi-well plate comprises a nanosensor (e.g. the nanosensor is in each of the wells or is the bottom or a part sidewall of each well). The capture agent in each well can be the same or different. In some embodiments, multiple different capture agents, each coated on different location can be placed in a well, which provide multiplexing of detections for different analyst. In these embodiments, several analytes in a sample may be analyzed in parallel. In some embodiments, the nanosensor can be a part of micro or nanofluidic channel.

In particular embodiments, a subject nanosensor may further comprise labeled analyte that is specifically bound to the capture agent. As noted above, the labeled analyte may be directly or indirectly labeled with a light-emitting label. In embodiments in which an analyte is indirectly labeled with a light-emitting label, the analyte may be bound to a second capture agent, also termed: detection agent (e.g., a secondary antibody or another nucleic acid) that is itself optically labeled. The second capture agent may be referred to as a “detection agent” in some cases.

In other embodiments, a subject nanosensor may be disposed inside a microfluidic channel (channel width of 1 to 1000 micrometers) or nanofluidic channel (channel width less 1 micrometer) or a part of inside wall of such channels. The nanosensors may be disposes at multiple locations inside each channel and be used in multiple channels. The nanosensors in different locations or different fluidic channels may later coated with different capture agents for multiplexing of detections.

A sensor may also include a molecular adhesion layer that covers at least a part of said metallic dot structure, said metal disc, and/or said metallic back plane and, optionally, a capture agent that specifically binds to a biomarker, wherein said capture agent is linked to the molecular adhesion layer of the sensor. The term “molecular adhesion layer” refers to a layer or multilayer of molecules of defined thickness that comprises an inner surface that is attached to the nanodevice and an outer (exterior) surface can be bound to capture agents. The molecular adhesion layer (MAL) can have many different configurations, including (a) a self-assembled monolayer (SAM) of cross-link molecules, (b) a multi-molecular layers thin film, (c) a combination of (a) and (b), and (d) a capture agent itself. The D2PA can amplify a light signal from an analyte, when said analyte is bound to the capture agent. One preferred D2PA embodiment is that the dimension of one, several or all critical metallic and dielectric components of sensor are less than the wavelength of the light in sensing. Details of the physical structure of disk-coupled dots-on-pillar antenna arrays, methods for their fabrication, methods for linking capture agents to disk-coupled dots-on-pillar antenna arrays and methods of using disk-coupled dots-on-pillar antenna arrays to detect analytes are described in a variety of publications including WO2012024006, WO2013154770, Li et al (Optics Express 2011 19, 3925-3936), Zhang et al (Nanotechnology 2012 23: 225-301); and Zhou et al (Anal. Chem. 2012 84: 4489) which are incorporated by reference for those disclosures.

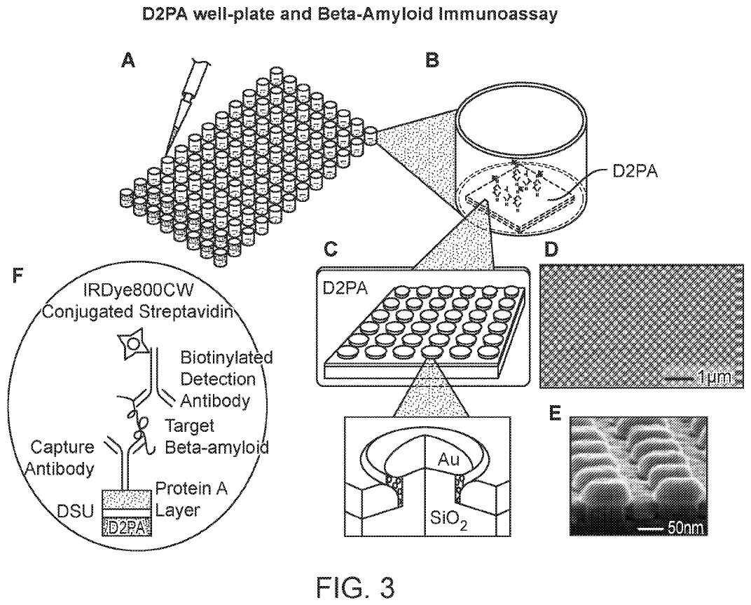

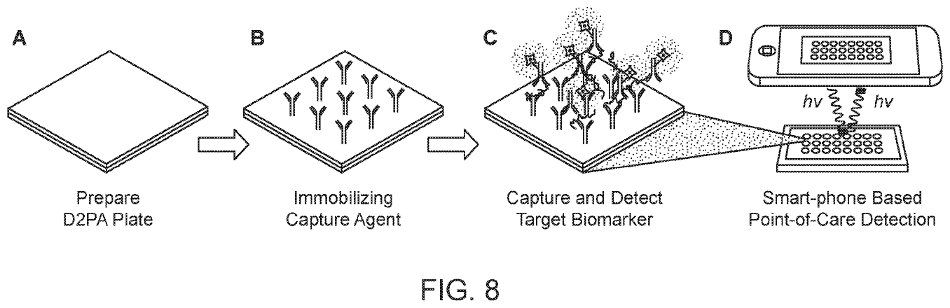

In certain embodiments, the sensor contains a capture agent that binds to an analyte of interest in a sample, as described in further detail above. The capture agent may vary depending on the analyte of interest to be detected in a sample. In some cases, the capture agent is an antibody, an antibody epitope, a nucleic acid binding protein, a nucleic acid, etc., as discussed above. In some embodiments, the capture agent is stably bound to the exterior surface of the D2PA molecular adhesion layer by reacting with a capture-agent-reactive group, i.e., a reactive group that can chemically react with capture agents, e.g., an amine-reactive group, a thiol-reactive group, a hydroxyl-reactive group, an imidazolyl-reactive group and a guanidinyl-reactive group, etc. (FIGS. 3 and 8.)

In an embodiment of MAL, where the molecular adhesion layer is a self-assembled monolayer (SAM) of cross-link molecules or ligands, each molecule for the SAM comprises of three parts: (i) head group, which has a specific chemical affinity to the nanodevice's surface, (ii) terminal group, which has a specific affinity to the capture agent, and (iii) molecule chain, which is a long series of molecules that link the head group and terminal group, and its length (which determines the average spacing between the metal to the capture agent) can affect the light amplification of the nanodevice.

In many embodiments, the head group attached to the metal surface belongs to the thiol group, e.t., —SH. Other alternatives for head groups that attach to metal surface are, carboxylic acid (—COOH), amine (C═N), selenol (—SeH), or phosphane (—P). Other head groups, e.g. silane (—SiO), can be used if a monolayer is to be coated on dielectric materials or semiconductors, e.g., silicon.

In many embodiments, the terminal groups can comprise a variety of capture agent-reactive groups, including, but not limited to, N-hydroxysuccinimidyl ester, sulfo-N-hydroxysuccinimidyl ester, a halo-substituted phenol ester, pentafluorophenol ester, a nitro-substituted phenol ester, an anhydride, isocyanate, isothiocyanate, an imidoester, maleimide, iodoacetyl, hydrazide, an aldehyde, or an epoxide. Other suitable groups are known in the art and may be described in, e.g., Hermanson, “Bioconjugate Techniques” Academic Press, 2nd Ed., 2008. The terminal groups can be chemically attached to the molecule chain after they are assembled to the nanodevice surface, or synthesized together with the molecule chain before they are assembled on the surface.

Other terminal groups are carboxyl —COOH groups (activated with EDC/NHS to form covalent binding with —NH2 on the ligand); Amine, —NH2, group (forming covalent binding with —COOH on the ligand via amide bond activated by EDC/NHS); Epoxy, Reacted with the —NH2 (the ligand without the need of a cross-linker); Aldehyde, (Reacted with the —NH2 on the ligand without the need of a cross-linker); Thiol, —SH, (link to —NH2 on the ligand through SMCC-like bioconjugation approach); and Glutathione, (GHS) (Ideal for capture of the GST-tagged proteins.

In one embodiment, streptavidin (SA) itself can be use as a functional group (e.g. terminal group) the SAM to crosslink capture agent molecules that have high binding affinity to SA, such as biotinylated molecules, including peptides, oligonucleotides, proteins and sugars.

The functional group of avidin, streptavidin have a high affinity to the biotin group to form avidin-biotin. Such high affinity makes avidin/streptavidin serve well as a functional group and the biotin group as complementray functional group binding. Such functional group can be used in binding the molecular adhesion layer to the nanodevice, in binding between molecular adhesion layer and the capture agent, and in binding a light emitting lable to the secondary capture agent. In one embodiment, a molecular adhesion layer containing thiol-reactive groups may be made by linking a gold surface to an amine-terminated SAM, and further modifying the amine groups using sulfosuccinimidyl-4-(N-maleimidomethyl)cyclohexane-1-carboxylate (Sulfo-SMCC) to yield a maleimide-activated surface. Maleimide-activated surfaces are reactive thiol groups and can be used to link to capture agents that contain thiol- (e.g., cysteine) groups.

Capture agents can be attached to the molecular adhesion layer via any convenient method such as those discussed above. Further methods of attaching capture agents to the molecular adhesion layer is described in, e.g., PCT App. Pub. No. WO2013154770, which is incorporated herein by reference. In many cases, a capture agent may be attached to the molecular adhesion layer via a high-affinity strong interactions such as those between biotin and streptavidin. Because streptavidin is a protein, streptavidin can be linked to the surface of the molecular adhesion layer using any of the amine-reactive methods described above. Biotinylated capture agents can be immobilized by spotting them onto the streptavidin. In other embodiments, a capture agent can be attached to the molecular adhesion layer via a reaction that forms a stong bond, e.g., a reaction between an amine group in a lysine residue of a protein or an aminated oligonucleotide with an NHS ester to produce an amide bond between the capture agent and the molecular adhesion layer. In other embodiment, a capture agent can be strongly attached to the molecular adhesion layer via a reaction between a sulfhydryl group in a cysteine residue of a protein or a sulfhydrl-oligonucleotide with a sulfhydryl-reactive maleimide on the surface of the molecular adhesion layer. Protocols for linking capture agents to various reactive groups are well known in the art.

In one embodiment, capture agent can be nucleic acid to capture proteins, or capture agent can be proteins that capture nucleic acid, e.g., DNA, RNA. Nucleic acid can bind to proteins through sequence-specific (tight) or non-sequence specific (loose) bond.

### Utility

The subject method finds use in a variety of different applications where determination of the presence or absence, and/or quantification of one or more analytes in a sample are desired. For example, the subject method finds use in the detection of proteins, peptides, nucleic acids, synthetic compounds, inorganic compounds, and the like.

In certain embodiments, the subject method finds use in the detection of nucleic acids, proteins, or other biomolecules in a sample. The methods may include the detection of a set of biomarkers, e.g., two or more distinct protein or nucleic acid biomarkers, in a sample. For example, the methods may be used in the rapid, clinical detection of two or more disease biomarkers in a biological sample, e.g., as may be employed in the diagnosis of a disease condition in a subject, or in the ongoing management or treatment of a disease condition in a subject, etc. As described above, communication to a physician or other health-care provider may better ensure that the physician or other health-care provider is made aware of, and cognizant of, possible concerns and may thus be more likely to take appropriate action.

The applications of the present method of employing a signal-amplifying nanosensor include, but are not limited to, (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals. Some of the specific applications of the present method are described now in further detail.

### Diagnostic Method

In certain embodiments, the subject method finds use in detecting biomarkers. In some cases, the present method may be used to detect the presence or absence of particular biomarkers, as well as an increase or decrease in the concentration of particular biomarkers in blood, plasma, serum, or other bodily fluids or excretions, such as but not limited to urine, blood, serum, plasma, saliva, semen, prostatic fluid, nipple aspirate fluid, lachrymal fluid, perspiration, feces, cheek swabs, cerebrospinal fluid, cell lysate samples, amniotic fluid, gastrointestinal fluid, biopsy tissue, and the like. Thus, the sample, e.g. a diagnostic sample, may include various fluid or solid samples. In some instances, the sample can be a bodily fluid sample from a subject who is to be diagnosed. In some instances, solid or semi-solid samples can be provided. The sample can include tissues and/or cells collected from the subject. The sample can be a biological sample. Examples of biological samples can include but are not limited to, blood, serum, plasma, a nasal swab, a nasopharyngeal wash, saliva, urine, gastric fluid, spinal fluid, tears, stool, mucus, sweat, earwax, oil, a glandular secretion, cerebral spinal fluid, tissue, semen, vaginal fluid, interstitial fluids derived from tumorous tissue, ocular fluids, spinal fluid, a throat swab, breath, hair, finger nails, skin, biopsy, placental fluid, amniotic fluid, cord blood, lymphatic fluids, cavity fluids, sputum, pus, microbiota, meconium, breast milk, exhaled condensate and/or other excretions. The samples may include nasopharyngeal wash. Nasal swabs, throat swabs, stool samples, hair, finger nail, ear wax, breath, and other solid, semi-solid, or gaseous samples may be processed in an extraction buffer, e.g., for a fixed or variable amount of time, prior to their analysis. The extraction buffer or an aliquot thereof may then be processed similarly to other fluid samples if desired. Examples of tissue samples of the subject may include but are not limited to, connective tissue, muscle tissue, nervous tissue, epithelial tissue, cartilage, cancerous sample, or bone.

In some instances, the subject from which a diagnostic sample is obtained may be a healthy individual, or may be an individual at least suspected of having a disease or a health condition. In some instances, the subject may be a patient.

In certain embodiments, the signal-amplifying nanosensor includes a capture agent configured to specifically bind a biomarker in a sample provided by the subject. In certain embodiments, the biomarker may be a protein. In certain embodiments, the biomarker protein is specifically bound by an antibody capture agent present in the signal-amplifying nanosensor. In certain embodiments, the biomarker is an antibody specifically bound by an antigen capture agent present in the signal-amplifying nanosensor. In certain embodiments, the biomarker is a nucleic acid specifically bound by a nucleic acid capture agent that is complementary to one or both strands of a double-stranded nucleic acid biomarker, or complementary to a single-stranded biomarker. In certain embodiments, the biomarker is a nucleic acid specifically bound by a nucleic acid binding protein. In certain embodiments, the biomarker is specifically bound by an aptamer.

The presence or absence of a biomarker or significant changes in the concentration of a biomarker can be used to diagnose disease risk, presence of disease in an individual, or to tailor treatments for the disease in an individual. For example, the presence of a particular biomarker or panel of biomarkers may influence the choices of drug treatment or administration regimes given to an individual. In evaluating potential drug therapies, a biomarker may be used as a surrogate for a natural endpoint such as survival or irreversible morbidity. If a treatment alters the biomarker, which has a direct connection to improved health, the biomarker can serve as a surrogate endpoint for evaluating the clinical benefit of a particular treatment or administration regime. Thus, personalized diagnosis and treatment based on the particular biomarkers or panel of biomarkers detected in an individual are facilitated by the subject method. Furthermore, the early detection of biomarkers associated with diseases is facilitated by the high sensitivity of the present method, as described above. Due to the capability of detecting multiple biomarkers with a mobile device, such as a smartphone, combined with sensitivity, scalability, and ease of use, the presently disclosed method finds use in portable and point-of-care or near-patient molecular diagnostics.

In certain embodiments, the subject method finds use in detecting biomarkers for a disease or disease state. In certain instances, the subject method finds use in detecting biomarkers for the characterization of cell signaling pathways and intracellular communication for drug discovery and vaccine development. For example, the subject method may be used to detect and/or quantify the amount of biomarkers in diseased, healthy or benign samples. In certain embodiments, the subject method finds use in detecting biomarkers for an infectious disease or disease state. In some cases, the biomarkers can be molecular biomarkers, such as but not limited to proteins, nucleic acids, carbohydrates, small molecules, and the like.

The subject method find use in diagnostic assays, such as, but not limited to, the following: detecting and/or quantifying biomarkers, as described above; screening assays, where samples are tested at regular intervals for asymptomatic subjects; prognostic assays, where the presence and or quantity of a biomarker is used to predict a likely disease course; stratification assays, where a subject's response to different drug treatments can be predicted; efficacy assays, where the efficacy of a drug treatment is monitored; and the like.

In some embodiments, a subject biosensor can be used diagnose a pathogen infection by detecting a target nucleic acid from a pathogen in a sample. The target nucleic acid may be, for example, from a virus that is selected from the group comprising human immunodeficiency virus 1 and 2 (HIV-1 and HIV-2), human T-cell leukaemia virus and 2 (HTLV-1 and HTLV-2), respiratory syncytial virus (RSV), adenovirus, hepatitis B virus (HBV), hepatitis C virus (HCV), Epstein-Barr virus (EBV), human papillomavirus (HPV), varicella zoster virus (VZV), cytomegalovirus (CMV), herpes-simplex virus 1 and 2 (HSV-1 and HSV-2), human herpesvirus 8 (HHV-8, also known as Kaposi sarcoma herpesvirus) and flaviviruses, including yellow fever virus, dengue virus, Japanese encephalitis virus, West Nile virus and Ebola virus. The present invention is not, however, limited to the detection of nucleic acid, e.g., DNA or RNA, sequences from the aforementioned viruses, but can be applied without any problem to other pathogens important in veterinary and/or human medicine.

Human papillomaviruses (HPV) are further subdivided on the basis of their DNA sequence homology into more than 70 different types. These types cause different diseases. HPV types 1, 2, 3, 4, 7, 10 and 26-29 cause benign warts. HPV types 5, 8, 9, 12, 14, 15, 17 and 19-25 and 46-50 cause lesions in patients with a weakened immune system. Types 6, 11, 34, 39, 41-44 and 51-55 cause benign acuminate warts on the mucosae of the genital region and of the respiratory tract. HPV types 16 and 18 are of special medical interest, as they cause epithelial dysplasias of the genital mucosa and are associated with a high proportion of the invasive carcinomas of the cervix, vagina, vulva and anal canal. Integration of the DNA of the human papillomavirus is considered to be decisive in the carcinogenesis of cervical cancer. Human papillomaviruses can be detected for example from the DNA sequence of their capsid proteins L1 and L2. Accordingly, the method of the present invention is especially suitable for the detection of DNA sequences of HPV types 16 and/or 18 in tissue samples, for assessing the risk of development of carcinoma.

Other pathogens that may be detected in a diagnostic sample using the present method include, but are not limited to: Varicella zoster; Staphylococcus epidermidis, Escherichia coli, methicillin-resistant Staphylococcus aureus (MSRA), Staphylococcus aureus, Staphylococcus hominis, Enterococcus faecalis, Pseudomonas aeruginosa, Staphylococcus capitis, Staphylococcus warneri, Klebsiella pneumoniae, Haemophilus influenzae, Staphylococcus simulans, Streptococcus pneumoniae and Candida albicans; gonorrhea (Neisseria gorrhoeae), syphilis (Treponena pallidum), clamydia (Chlamydia tracomitis), nongonococcal urethritis (Ureaplasm urealyticum), chancroid (Haemophilus ducreyi), trichomoniasis (Trichomonas vaginalis); Pseudomonas aeruginosa, methicillin-resistant Staphlococccus aureus (MSRA), Klebsiella pneumoniae, Haemophilis influenzae, Staphylococcus aureus, Stenotrophomonas maltophilia, Haemophilis parainfluenzae, Escherichia coli, Enterococcus faecalis, Serratia marcescens, Haemophilis parahaemolyticus, Enterococcus cloacae, Candida albicans, Moraxiella catarrhalis, Streptococcus pneumoniae, Citrobacter freundii, Enterococcus faecium, Klebsella oxytoca, Pseudomonas fluorscens, Neiseria meningitidis, Streptococcus pyogenes, Pneumocystis carinii, Klebsella pneumoniae Legionella pneumophila, Mycoplasma pneumoniae, and Mycobacterium tuberculosis, etc., as well as those listed in Tables 2 and 6.

In some cases, the signal-amplifying nanosensor may be employed to detect a biomarker that is present at a low concentration. For example, the signal-amplifying nanosensor may be used to detect cancer antigens in a readily accessible bodily fluids (e.g., blood, saliva, urine, tears, etc.), to detect biomarkers for tissue-specific diseases in a readily accessible bodily fluid (e.g., a biomarkers for a neurological disorder (e.g., Alzheimer's antigens)), to detect infections (particularly detection of low titer latent viruses, e.g., HIV), to detect fetal antigens in maternal blood, and for detection of exogenous compounds (e.g., drugs or pollutants) in a subject's bloodstream, for example.

The following Tables 1-3 provide lists of biomarkers that can be detected using the subject signal-amplifying nanosensor (when used in conjunction with an appropriate monoclonal antibody, nucleic acid, or other capture agent), and their associated diseases. One potential source of the biomarker (e.g., “CSF”; cerebrospinal fluid) is also indicated in the table. In many cases, the subject biosensor can detect those biomarkers in a different bodily fluid to that indicated. For example, biomarkers that are found in CSF can be identified in urine, blood or saliva. It will also be clear to one with ordinary skill in the art that the subject signal-amplifying nanosensors may be configured to capture and detect many more biomarkers known in the art that are diagnostic of a disease or health condition.

A biomarker, as listed in the tables provided herein, may be a protein or a nucleic acid (e.g., mRNA) biomarker, unless specified otherwise. The diagnosis may be associated with an increase or a decrease in the level of a biomarker in the sample, unless specified otherwise.

TABLE 1
Diagnostic Markers

| Marker | disease |
|---|---|
| Aβ42, amyloid beta-protein (CSF) | Alzheimer's disease. |
| fetuin-A (CSF) | multiple sclerosis. |
| tau (CSF) | niemann-pick type C. |
| secretogranin II (CSF) | bipolar disorder. |
| prion protein (CSF) | Alzheimer disease, prion disease |
| Cytokines (CSF) | HIV-associated neurocognitive disorders |
| Alpha-synuclein (CSF) | parkinsonian disorders (neuordegenerative |
| | disorders) |
| tau protein (CSF) | parkinsonian disorders |
| neurofilament light chain (CSF) | axonal degeneration |
| parkin (CSF) | neuordegenerative disorders |
| PTEN induced putative kinase 1 (CSF) | neuordegenerative disorders |
| DJ-1 (CSF) | neuordegenerative disorders |
| leucine-rich repeat kinase 2 (CSF) | neuordegenerative disorders |
| mutated ATP13A2 (CSF) | Kufor-Rakeb disease |
| Apo H (CSF) | parkinson disease (PD) |
| ceruloplasmin (CSF) | PD |
| Peroxisome proliferator-activated receptor | PD |
| gamma coactivator-1 alpha (PGC-1α)(CSF) | |
| transthyretin (CSF) | CSF rhinorrhea (nasal surgery samples) |
| Vitamin D-binding Protein (CSF) | Multiple Sclerosis Progression |
| proapoptotic kinase R (PKR) and its | AD |
| phosphorylated PKR (pPKR) (CSF) | |
| CXCL13 (CSF) | multiple sclerosis |
| IL-12p40, CXCL13 and IL-8 (CSF) | intrathecal inflammation |
| Dkk-3 (semen) | prostate cancer |
| p14 endocan fragment (blood) | Sepsis: Endocan, specifically secreted by |
| | activated-pulmonary vascular endothelial |
| | cells, is thought to play a key role in the |
| | control of the lung inflammatory reaction. |
| Serum (blood) | neuromyelitis optica |
| ACE2 (blood) | cardiovascular disease |
| autoantibody to CD25 (blood) | early diagnosis of esophageal squamous cell |
| | carcinoma |
| hTERT (blood) | lung cancer |
| CAI25 (MUC 16) (blood) | lung cancer |
| VEGF (blood) | lung cancer |
| sIL-2 (blood) | lung cancer |
| Osteopontin (blood) | lung cancer |
| Human epididymis protein 4 (HE4) (blood) | ovarian cancer |
| Alpha-Fetal Protein (blood) | pregnancy |
| Albumin (urine) | diabetics |
| albumin (urine) uria | albuminuria |
| microalbuminuria | kidney leaks |
| AFP (urine) | mirror fetal AFP levels |
| neutrophil gelatinase-associated lipocalin (NGAL) | Acute kidney injury |
| (urine) | |
| interleukin 18 (IL-18) (urine) | Acute kidney injury |
| Kidney Injury Molecule -1 (KIM-1) (urine) | Acute kidney injury |
| Liver Fatty Acid Binding Protein (L-FABP) (urine) | Acute kidney injury |
| LMP1 (saliva) | Epstein-Barr virus oncoprotein |
| | (nasopharyngeal carcinomas) |
| BARF1 (saliva) | Epstein-Barr virus oncoprotein |
| | (nasopharyngeal carcinomas) |
| IL-8 (saliva) | oral cancer biomarker |
| carcinoembryonic antigen (CEA) (saliva) | oral or salivary malignant tumors |
| BRAF, CCNI, EGRF, FGF19, FRS2, GREB1, and | Lung cancer |
| LZTS1 (saliva) | |
| alpha-amylase (saliva) | cardiovascular disease |
| carcinoembryonic antigen (saliva) | Malignant tumors of the oral cavity |
| CA 125 (saliva) | Ovarian cancer |
| IL8 (saliva) | spinalcellular carcinoma. |
| thioredoxin (saliva) | spinalcellular carcinoma. |
| beta-2 microglobulin levels - monitor activity of | HIV |
| the virus (saliva) | |
| tumor necrosis factor-alpha receptors - monitor | HIV |
| activity of the virus (saliva) | |
| CA15-3 (saliva) | breast cancer |

TABLE 2
Diagnostic Markers

| Disease/Condition | Source | Biomarker |
|---|---|---|
| HPA axis activity | Saliva | Cortisol |
| (Cushing's disease, | | |
| Adrenal cortex | | |
| diseases, etc.) | | |
| Pregnancy/fetal | Saliva | progesterone |
| development | urine | human chorionic gonadotropin, |
| | | Levonorgestrel, alpha-fetoprotein, early |
| | | conception factor, Unconjugated Estriol |
| | serum | Estradiol, interleukin-6, Unconjugated |
| | | Estriol, Inhibin-A |
| Infant development | urine | NGAL, KIM-1, Cys-C, and B2mG, AFP |
| | | S100B, MBP |
| Menopause | Saliva | Follicle stimulating hormone (FSH) |
| | | Estrogen and progesterone, testosterone, |
| | | free testosterone, and |
| | | dehydroepiandrosterone sulfate (DHEAS), |
| | | cortisol and dehydroepiandrosterone |
| | | (DHEA) |
| Polycystic ovary | saliva | testosterone |
| syndrome | | |
| Andropause | saliva | testosterone; testosterone precursors such |
| | | as pregnenolone, progesterone, 17- |
| | | hydroxypregnenolone, 17- |
| | | hydroxyprogesterone, |
| | | dehydroepiandrosterone (DHEA) and |
| | | delta-4-androstene-3,17-dione; testosterone |
| | | and dihydrotestosterone metabolites such |
| | | as the 17-ketosteroids androsterone and |
| | | etiocholanolone, polar metabolites in the |
| | | form of diols, triols, and conjugates, as |
| | | well estradiol, estrogens, androsteindione, |
| | | cortisol, DHEA, FSH (follicle stimulating |
| | | hormone), LH (luteinizing hormone), and |
| | | GnRH (gonadotropin-releasing hormone) |
| Coagulation | miscellaneous | b-Thromboglobulin, Platelet factor 4, Von |
| status/disorders | | Willebrand factor, Factor I: Fibrinogen, |
| | | Factor II: Prothrombin, Factor III: Tissue |
| | | factor, Factor IV: Calcium, Factor V: |
| | | Proaccelerin, Factor VI, Factor VII: |
| | | Proconvertin, Factor VIII:, Anti-hemolytic |
| | | factor, Factor IX: Christmas factor, Factor |
| | | X: Stuart-Prower factor, Factor XI: Plasma |
| | | thromboplastin antecedent, Factor XII: |
| | | Hageman factor, Factor XIII: Fibrin- |
| | | stabilizing factor, Prekallikrein, High- |
| | | molecular-weight kininogen, Protein C, |
| | | Protein S, D-dimer, Tissue plasminogen |
| | | activator, Plasminogen, a2-Antiplasmin, |
| | | Plasminogen activator inhibitor 1 (PAI1) |
| Autism | miscellaneous | miR-484, miR-21, miR-212, miR-23a, |
| | | miR-598, miR-95, miR-129, miR-431, |
| | | miR-7, miR-15a, miR-27a, miR-15b, miR- |
| | | 148b, miR-132, or miR-128; miR-93, miR- |
| | | 106a, miR-539, miR-652, miR-550, miR- |
| | | 432, miR-193b, miR-181d, miR-146b, |
| | | miR-140, miR-381, miR-320a, or miR- |
| | | 106b; GM1, GD1a, GD1b, or GT1b |
| | | Ceruloplasmin, Metalothioneine, Zinc, |
| | | Copper, B6, B12, Glutathione, Alkaline |
| | | phosphatase, and Activation of apo- |
| | | alkaline phosphatases |
| Alzheimer's Disease | miscellaneous | miR-107, miR-29a, miR-29b-1, or miR-9; |
| | | miR-128; HIF-1α, BACE1, Reelin, |
| | | CHRNA7, or 3Rtau/4Rtau; BACE1, |
| | | Reelin, Cystatin C, Truncated Cystatin C, |
| | | Amyloid Beta, C3a, t-Tau, Complement |
| | | factor H, or alpha-2-macroglobulin |
| | CSF, serum, | β-amyloid(1-42), β-amyloid(1-40), tau, |
| | saliva | phosphor-tau-181 |
| | Saliva | cTnI, myoglobin, MMP-9, MMP-8, MMP- |
| | | 2, sICAM-1, myeloperoxidase [MPO], IL- |
| | | 4, and/or IL-5; B-type natiuretic peptide |
| | | [BNP], IL-1α, IL-11, IL-10, TNF-α, IFN-γ, |
| | | VEGF, insulin, GLP-1 (active), GLP-1 |
| | | (total), TREM1, Leukotriene E4, Akt1, Aβ- |
| | | 40, Aβ-42, Fas ligand, PSA, G-CSF, MIP- |
| | | 1α, IL-22, IL-8, IL-21, IL-15, IL-6, IL-7, |
| | | GM-CSF, IL-2, IL-12, IL-17α, IL-1β, |
| | | MCP, IL-32 or RANTES, apolipoproteins |
| | | A1, D and E, ischemia-modified albumin |
| | | (IMA), fibronectin, s. alpha-amylase, |
| | | aspartate aminotransferase, lactate |
| | | dehydrogenase, tissue factor activity, |
| | | MCP-1, sVCAM-1, sCD-40, insulin-like |
| | | growth factor I (IGF-I), IGF-II |
| | | acetylcholinesterase enzyme (AChE), |
| | Serum/CSF | β-amyloid(1-42), β-amyloid(1-40), tau, |
| | | phosphor-tau-181, GSK-3, PKC, VCAM-1 |
| | | and ICAM-1, macrophage inflammatory |
| | | proteins-1δ and -4 (MIP1δ and MIP4), |
| | | regulated upon activation normal T-cell |
| | | (RANTES), tumor necrosis factor-alpha |
| | | (TNFα), midregional pro-atrial natriuretic |
| | | peptide (MR-proANP) |
| | | AD-associated neuronal thread protein |
| | | (AD7c-NTP) |
| Parkinson's Disease | miscellaneous | miR-133b; Nurrl, BDNF, TrkB, gstm1, or |
| | | 5100 beta; apo-H, Ceruloplasmin, BDNF, |
| | | IL-8, Beta2-microglobulin, apoAII, tau, |
| | | ABeta1-42, DJ-1 |
| | Saliva | cTnI, myoglobin, MMP-9, MMP-8, MMP- |
| | | 2, sICAM-1, myeloperoxidase [MPO], IL- |
| | | 4, and/or IL-5; B-type natiuretic peptide |
| | | [BNP], IL-1α, IL-11, IL-10, TNF-β, IFN-γ, |
| | | VEGF, insulin, GLP-1 (active), GLP-1 |
| | | (total), TREM1, Leukotriene E4, Akt1, Aβ- |
| | | 40, Aβ-42, Fas ligand, PSA, G-CSF, MIP- |
| | | 1α, IL-22, IL-8, IL-21, IL-15, IL-6, IL-7, |
| | | GM-CSF, IL-2, IL-12, IL-17α, IL-1β, |
| | | MCP, IL-32 or RANTES, apolipoproteins |
| | | A1, D and E, ischemia-modified albumin |
| | | (IMA), fibronectin, s. alpha-amylase, |
| | | aspartate aminotransferase, lactate |
| | | dehydrogenase, tissue factor activity, |
| | | MCP-1, sVCAM-1, sCD-40, insulin-like |
| | | growth factor I (IGF-I), IGF-II |
| Schizophrenia | miscellaneous | miR-181b; miR-7, miR-24, miR-26b, miR- |
| | | 29b, miR-30b, miR-30e, miR-92, or miR- |
| | | 195; IFITM3, SERPINA3, GLS, or |
| | | ALDH7A1BASP1; TP5B, ATP5H, |
| | | ATP6V1B, DNM1, NDUFV2, NSF, |
| | | PDHB |
| Bipolar disease | miscellaneous | FGF2, ALDH7A1, AGXT2L1, AQP4, or |
| | | PCNT2 |
| Mood disorder | (blood) | Mbp, Edg2, Fgfr1, Fzd3, Mag, Pmp22, |
| | | Ugt8, Erbb3, Igfbp4, Igfbp6, Pde6d, Ptprm, |
| | | Nefh, Atp2c1, Atxn1, Btg1, C6orf182, |
| | | Dicer1, Dnajc6, and Ednrb |
| Major Depressive | miscellaneous | FGFR1, FGFR2, FGFR3, or AQP4 |
| Disorder | | Secretogranin, VGF |
| | serum | Cortisol; EGF; GCS; PPY; ACTH; |
| | | AVP; CRH; A1AT; A2M; ApoC3; |
| | | CD40L; IL-6; IL-13; IL-18; IL-1ra; |
| | | MPO; PAI-1; TNFA; ACRP30; ASP; |
| | | FABP; INS; LEP; PRL; RETN; |
| | | Testosterone; TSH; BDNF; S100B; |
| | | NTF3; GDNF; ARTN |
| Prion disease | miscellaneous | Amyloid B4, App, IL-1R1, or SOD1; |
| | | PrP(c), 14-3-3, NSE, S-100, Tau, AQP-4 |
| Inflammation | miscellaneous | TNF-α, IL-6, IL1β, Rheumatoid factor |
| | | (RF), Antinuclear Antibody (ANA), acute |
| | | phase markers including C-reactive protein |
| | | (CRP), Clara Cell Protein (Uteroglobin) |
| Multiple sclerosis | miscellaneous | 14-3-3 protein epsilon; Isoform Long of |
| | | Protocadherin alpha C2 precursor; Insulin- |
| | | like growth factor IA precursor; Isoform 1 |
| | | of Protocadherin-8 precursor; Isoform 1 of |
| | | Sodium/potassium/calcium exchanger 2 |
| | | precursor; Complement factor H-related 5; |
| | | Di-N-acetylchitobiase precursor; Isoform 1 |
| | | of Protein NDRG2; N-acetylglucosamine- |
| | | 6-sulfatase precursor; Isoform 1 of |
| | | Semaphorin-3B precursor; Cadherin-5 |
| | | precursor; UPF0454 protein C12orf49 |
| | | precursor; Dihydrolipoyl dehydrogenase, |
| | | mitochondrial precursor; Metallothionein- |
| | | 3; Fas apoptotic inhibitory molecule 2; |
| | | Coactosin-like protein; Isoform Long of |
| | | Platelet-derived growth factor A chain |
| | | Precursor; Isoform Long of Endothelin-3 |
| | | precursor; HLA class I histocompatibility |
| | | antigen, A-1 alpha chain Precursor; |
| | | Neuronal pentraxin-2 precursor; retbindin |
| | | isoform 2; Neuroendocrine convertase 2 |
| | | precursor; 15 kDa selenoprotein isoform 1 |
| | | precursor; Phospholipase D4; Isoform 1 of |
| | | CD109 antigen precursor; Ectonucleotide |
| | | pyrophosphatase/phosphodiesterase family; |
| | | member 6 precursor; Fascin; Golgi |
| | | phosphoprotein 2; Isoform Delta 6 of |
| | | Calcium/calmodulin-dependent protein |
| | | kinase type II delta chain; Isoform 1 of |
| | | FRAS1-related extracellular matrix protein |
| | | 2 Precursor; Putative uncharacterized |
| | | protein LOC130576; Isoform 1 of L-lactate |
| | | dehydrogenase A chain; Isoform 1 of |
| | | Polypeptide N- |
| | | acetylgalactosaminyltransferase 13; |
| | | Papilin; Protein DJ-1; Beta-mannosidase |
| | | precursor; Protein YIPF3; Isoform 1 of |
| | | Receptor-type tyrosine-protein phosphatase |
| | | N2 Precursor; Cell growth regulator with |
| | | EF hand domain protein 1; Sulfhydryl |
| | | oxidase 2 precursor; Ig lambda chain V-II |
| | | region TRO; Ig lambda chain V-VI region |
| | | AR; Ig heavy chain V-III region WEA; Ig |
| | | heavy chain V-III region CAM; Ig heavy |
| | | chain V-III region BUR; Myosin-reactive |
| | | immunoglobulin kappa chain variable |
| | | region (Fragment); Microfibrillar protein 2 |
| | | (Fragment); Ig kappa chain V-III region |
| | | IARC/BL41 precursor; Ig kappa chain V-I |
| | | region Kue; Ig kappa chain V-I region |
| | | Scw; Ig kappa chain V-III region B6; |
| | | IGLV6-57 protein; hypothetical protein |
| | | LOC402665; Isoform 1 of Proline-rich |
| | | acidic protein 1 precursor; Rheumatoid |
| | | factor RF-ET13; Rheumatoid factor D5 |
| | | heavy chain (Fragment); Uncharacterized |
| | | protein ENSP00000375027; |
| | | Uncharacterized protein |
| | | ENSP00000375043; Uncharacterized |
| | | protein ENSP00000375019; |
| | | Isoform 1 of Protocadherin-1 precursor; |
| | | Isoform 1 of Epithelial discoidin domain- |
| | | containing receptor 1 precursor; Serine |
| | | protease HTRA1 precursor; Isoform Delta |
| | | of Poliovirus receptor-related protein 1 |
| | | Precursor; chemokine (C—X—C motif) |
| | | ligand 16; Plastin-2; 14-3-3 protein |
| | | zeta/delta; Apolipoprotein C-II precursor; |
| | | Brain-specific angiogenesis inhibitor 1 |
| | | precursor; Semaphorin-3G precursor; |
| | | Follistatin-related protein 3 precursor; |
| | | Hepatocyte growth factor activator |
| | | precursor; Isoform 1 of Contactin- |
| | | associated protein-like 2 precursor; |
| | | Phosphoglycerate kinase 1; Gamma- |
| | | enolase; Phosphoglycerate mutase 2; Low |
| | | affinity immunoglobulin gamma Fc region |
| | | receptor III-A precursor; Isoform Beta of |
| | | Poliovirus receptor precursor; Serine |
| | | protease inhibitor Kazal-type 6 precursor; |
| | | Isoform 1 of Chordin precursor; Out at first |
| | | protein homolog precursor; Isoform 1 of |
| | | Carboxypeptidase B2 precursor; ROBO2 |
| | | isoform a Ig kappa chain V-III region |
| | | POM; Isoform 1 of Protein-L- |
| | | isoaspartate(D-aspartate) O- |
| | | Methyltransferase CDNA FLJ45296 fis, |
| | | clone BRHIP3003340, moderately similar |
| | | to Actin, alpha skeletal muscle 2; Isoform |
| | | 1 of RGM domain family member B |
| | | precursor; Carboxypeptidase N subunit 2 |
| | | precursor; Hypothetical LOC284297 |
| | miscellaneous | L-6, IL-17, PAR-3, IL-17, T1/ST2, JunD, |
| | | 5-LO, LTA4H, MBP, PLP, or alpha-beta |
| | | crystallin |
| | | antithrombin III; a-2 glycoprotein 1, zinc; |
| | | transthyretin (prealbumin); NADH |
| | | dehydrogenase (ubiquinone) 1 beta |
| | | subcomplex, 2; neurotrimin; orosomucoid |
| | | 1 precursor (α-1-acid glycoprotein-1); |
| | | leucine-rich α-2-glycoprotein; leucine-rich |
| | | repeat protein; α-1-antitrypsin |
| Chronique fatigue | saliva | cortisol |
| syndrome | saliva | Ig alpha-1 chain C region; Polymeric |
| | | immunoglobulin receptor; Protein S100- |
| | | A7; Cystatin-C; Cystatin-B; 14-3-3 protein |
| | | zeta/delta; Zinc-alpha-2-glycoprotein |
| | | (ZAG) |
| Sjogren's syndrome | saliva | IgA, IgG, IgM autoantibodies; IgA, |
| | | lactoferrin and beta2-microglobulin; |
| | | lysozyme C, and cystatin C, amylase and |
| | | carbonic anhydrase |
| | miscellaneous | Autoantibodies (SSA/Ro; LA/SS-B) |
| Systemic lupus | miscellaneous | Autoantibodies (CDC25B, APOBEC3G, |
| erythematosus (SLE) | | ARAF, BCL2A1, CLK1, CREB1, |
| | | CSNK1G1, CSNK2A1, CWC27, DLX4, |
| | | DPPA2, EFHD2, EGR2, ERCC2, EWSR1, |
| | | EZH2, FES, FOS, FTHL17, GEM, |
| | | GNA15, GNG4, HMGB2, HNRNPUL1, |
| | | HOXB6, ID2, IFI35, IGF2BP3, IGHG1, |
| | | JUNB, KLF6, LGALS7, LIN28A, MLLT3, |
| | | NFIL3, NRBF2, PABPC1, PATZ1, |
| | | PCGF2, PPP2CB, PPP3CC, PRM1, PTK2, |
| | | PTPN4, PYGB, RET, RPL18A, RPS7, |
| | | RRAS, SCEL, SH2B1, SMAD2, STAM, |
| | | TAF9, TIE1, UBA3, VAV1, WT1, ZAP70, |
| | | or ZNRD1) |
| | miscellaneous | Autoantibodies ((i) KIT, (ii) C6orf93, (iii) |
| | | RPL34, (iv) DOM3Z, (v) COPG2, (vi) |
| | | DNCL12, (vii) RRP41, (viii) FBXO9, (ix) |
| | | RALBP1, (x) PIAS2, (xi) EEF1D, (xii) |
| | | CONI, (xiii) KATNB1, (xiv) POLR2E, |
| | | (xv) CCT3, (xvi) KIAA0643, (xvii) |
| | | RPL37A, (xviii) GTF2H2, (xix) MAP2K5, |
| | | (xx) CDK3, (xxi) RPS6KA1, (xxii) |
| | | MARK4, (xxiii) MTO1, (xxiv) |
| | | MGC42105, (xxv) NFE2L2, (xxvi) |
| | | WDR45L, (xxvii) STK4, (xxviii) PFKFB3, |
| | | (xxix) NTRK3, (xxx) MLF1, (xxxi) |
| | | TRIM37, (xxxii) ACTL7B, (xxxiii) |
| | | RPL18A, (xxxiv) CKS1B, (xxxv) TUBA1, |
| | | (xxxvi) NME6, (xxxvii) SUCLA2, |
| | | (xxxviii) IGHG1, (xxxix) PRKCBP1, (x1) |
| | | BAG3, (xli) TCEB3, (xlii) RPL15, (xliii) |
| | | SSX4, (xliv) MAP2K7, (xlv) EEF1G, |
| | | (xlvi) RNF38, (xlvii) PHLDA2, (xlviii) |
| | | KCMF1, (xlix) NUBP2, (I) VPS45A) |
| | miscellaneous | Autoantibodies (SSA/Ro; dsDNA; Smith; |
| | | histones; thrombin) |
| CREST syndrome | | Autoantibodies (centromere) |
| Systemic sclerosis | miscellaneous | Autoantibodies (Type I topoisomerase) |
| Primary biliary | miscellaneous | Autoantibodies (nucleoporin 62, Sp100 |
| cirrhosis | | nuclear antigen, nucleoporin 210 kDa, |
| | | mitochondria) |
| cirrhosis | miscellaneous | NLT; NLT, HBsAG, AST, YKL-40, |
| | | Hyaluronic acid, TIMP-1, alpha 2 |
| | | macroglobulin, a-1-antitrypsin P1Z allele, |
| | | haptoglobin, or acid phosphatase ACP AC |
| autoimmune hepatitis | miscellaneous | Autoantibodies (Liver kidney microsomal |
| | | type 1, smooth muscle) |
| Celiac disease | miscellaneous | Autoantibodies (tTG, actin) |
| Celiac disease | saliva | Anti-IgA gliadin |
| Irritable Bowel | miscellaneous | REG1A, MMP3 |
| Syndrome (IBS) | | |
| Inflammatory bowel | miscellaneous | Trypsinogen IV, SERT; II-16, II-1beta, II- |
| disease (IBD) | | 12, TNF-alpha, interferon gamma, 11-6, |
| | | Rantes, MCP-1, Resistin, or 5-HT |
| Ulcerative colitis | miscellaneous | IFITM1 , IFITM3, STAT1, STAT3, TAP1, |
| | | PSME2, PSMB8, HNF4G, KLF5, AQP8, |
| | | APT2B1, SLC16A, MFAP4, CCNG2, |
| | | SLC44A4, DDAH1, TOB1, 231152_at, |
| | | MKNK1, CEACAM7*, 1562836_at, |
| | | CDC42SE2, PSD3, 231169_at, IGL@*, |
| | | GSN, GPM6B, CDV3*, PDPK1, ANP32E, |
| | | ADAM9, CDH1, NLRP2, 215777_at, |
| | | OSBPL1, VNN1, RABGAP1L, |
| | | PHACTR2, ASH1L, 213710_s_at, CDH1, |
| | | NLRP2, 215777_at, OSBPL1, VNN1, |
| | | RABGAP1L, PHACTR2, ASH1, |
| | | 213710_s_at, ZNF3, FUT2, IGHA1, |
| | | EDEM1, GPR171, 229713_at, |
| | | LOC643187, FLVCR1, SNAP23*, |
| | | ETNK1, LOC728411, POSTN, MUC12, |
| | | HOXA5, SIGLEC1, LARP5, PIGR, |
| | | SPTBN1, UFM1, C6orf62, WDR90, |
| | | ALDH1A3, F2RL1, IGHV1-69, DUOX2, |
| | | RAB5A, or CP; (P)ASCA |
| Hyperplastic Polyp | miscellaneous | SLC6A14, ARHGEF10, ALS2, IL1RN, |
| | | SPRy4, PTGER3, TRIM29, SERPINB5, |
| | | 1560327_at, ZAK, BAG4, TRIB3, TTL, |
| | | FOXQ1 |
| Psoriasis | miscellaneous | miR-146b, miR-20a, miR-146a, miR-31, |
| | | miR-200a, miR-17-5p, miR-30e-5p, miR- |
| | | 141, miR-203, miR-142-3p, miR-21, or |
| | | miR-106a; miR-125b, miR-99b, miR-122a, |
| | | miR-197, miR-100, miR-381, miR-518b, |
| | | miR-524, let-7e, miR-30c, miR-365, miR- |
| | | 133b, miR-10a, miR-133a, miR-22, miR- |
| | | 326, or miR-215; IL-20, VEGFR-1, |
| | | VEGFR-2, VEGFR-3, or EGR1; |
| Dermatitis | miscellaneous | Autoantibodies (eTG) |
| herpetiformis | | |
| Miller-Fisher | miscellaneous | Autoantibodies (ganglioside GQ1B) |
| Syndrome | | |
| Wegener's | miscellaneous | Autoantibodies (c-ANCA) |
| granulomatosis | | |
| Neuropathies | miscellaneous | Autoantibodies (ganglioside GD3, |
| | | ganglioside GM1) |
| microscopic | miscellaneous | Autoantibodies (p-ANCA) |
| polyangiitis | | |
| Polymyositis | miscellaneous | Autoantibodies (Signal recognition |
| | | particles) |
| scleromyositis | miscellaneous | Autoantibodies (exosome complex Signal |
| | | recognition particles) |
| myasthenia gravis | miscellaneous | Autoantibodies (nicotinic acetylcholine |
| | | receptor Signal recognition particles, |
| | | muscle-specific kinase (MUSK) Signal |
| | | recognition particles) |
| Lambert-Eaton | miscellaneous | Autoantibodies (voltage-gated calcium |
| myasthenic syndrome | | channel (P/Q-type)) |
| Hashimoto's | miscellaneous | Autoantibodies (thyroid peroxidase) |
| thyroiditis | | |
| Graves' disease | miscellaneous | Autoantibodies (TSH receptor) |
| paraneoplastic | miscellaneous | Autoantibodies (Hu, Yo (cerebellar |
| cerebellar syndrome | | Purkinje Cells), amphiphysin) |
| encephalitis | miscellaneous | Autoantibodies (voltage-gated potassium |
| | | channel (VGKC), N-methyl-D-aspartate |
| | | receptor (NMDA)) |
| Sydenham's chorea | miscellaneous | Autoantibodies (basal ganglia neurons) |
| Neuromyelitis | miscellaneous | Autoantibodies (aquaporin-4) |
| Allergies | saliva | Allergen-specific IgAs |
| Rheumatic disease | miscellaneous | miR-146a, miR-155, miR-132, miR-16, or |
| | | miR-181; HOXD10, HOXD11, HOXD13, |
| | | CCL8, LIM homeobox2, or CENP-E; |
| | | TNFα |
| Rheumatoid arthritis | miscellaneous | Autoantibodies (Rheumatoid factor, cyclic |
| | | citrullinated protein) |
| Rheumatoid arthritis | miscellaneous | ATP-binding cassette, sub-family A, |
| | | member 12 isoform b; ATP-binding |
| | | cassette A12; apolipoprotein; B-100 |
| | | precursor - human; complement |
| | | component 3 precursor; alpha-2- |
| | | glycoprotein 1, zinc; Alpha-2-glycoprotein, |
| | | zinc; serine (or cysteine) proteinase |
| | | inhibitor, clade A (alpha-1 antiproteinase, |
| | | antitrypsin), member 2; Protease inhibitor |
| | | 1-like; protease inhibitor 1 (alpha-1- |
| | | antitrypsin)-like; group-specific component |
| | | (vitamin D binding protein); hDBP; serine |
| | | (or cysteine) proteinase inhibitor, clade A |
| | | (alpha-1 antiproteinase, antitrypsin), |
| | | member 1; Protease inhibitor (alpha-1- |
| | | antitrypsin); protease inhibitor 1 (anti- |
| | | elastase), alpha-1-antitrypsin; Vitronectin |
| | | precursor V65 subunit; A kinase anchor |
| | | protein 9 isoform 2; retrovirus-related |
| | | hypothetical protein II - human |
| | | retrotransposon LINE-1; nuclear receptor |
| | | coactivator RAP250; peroxisome |
| | | proliferator-act; nuclear receptor |
| | | coactivator RAP2; Ig kappa chain NIG26 |
| | | precursor - human; Vitamin D-binding |
| | | protein precursor (DBF) (Group-specific |
| | | component) (GC-globulin) (VDB) |
| | | complement C4A precursor [validated] |
| | | Human; guanine nucleotide binding protein |
| | | (G protein), gamma transducing activity |
| | | polypeptide 1; nucleoporin 98 kD isoform |
| | | 4; nucleoporin 98 kD; Nup98-Nup96 |
| | | precursor; GLFG-repeat containing; |
| | | nucleoporin; vitronectin precursor; serum |
| | | spreading factor; somatomedin B; |
| | | complement S-protein; Alpha-1-antitrypsin |
| | | precursor; HMG-BOX transcription; factor |
| | | BBX; x 001; protein; hect domain and |
| | | RLD 2; calcium channel, voltage- |
| | | dependent, L type, alpha 1C subunit; |
| | | Alpha-2-antiplasmin precursor (Alpha-2- |
| | | plasmin inhibitor) (Alpha-2-PI) (Alpha-2- |
| | | AP); Neuronal PAS domain protein 2 |
| | | (Neuronal PAS2) (Member of PAS protein |
| | | 4) (MOP4); Retinoic acid receptor gamma- |
| | | 2 (RAR-gamma-2) alpha-1-B-glycoprotein- |
| | | human; Heparin cofactor II precursor |
| | | (HC-II) (Protease inhibitor leuserpin 2) |
| | | (HLS2); Ig gamma-1 chain C region; |
| | | isocitrate dehydrogenase 3 (NAD+) alpha |
| | | precursor; H-IDH alpha; isocitric |
| | | dehydrogenase; isocitrate dehydrogenase |
| | | [NAD] sub-unit alpha, mitochondrial; |
| | | NAD+-specific ICDH; NAD(H)-specific |
| | | isocitrate dehydrogenase alpha subunit |
| | | precursor; isocitrate dehydrogenase |
| | | (NAD+) alpha chain precursor; ferroxidase |
| | | (EC 1.16.3.1) precursor [validated]- |
| | | human; similar to zona pellucida binding |
| | | protein; N-acetylneuraminic acid |
| | | phosphate synthase; sialic acid synthase; |
| | | sialic acid phosphate synthase; triple |
| | | functional domain (PTPRF interacting); |
| | | deleted in bladder cancer chromosome |
| | | region candidate 1; ceruloplasmin |
| | | (ferroxidase); Ceruloplasmin; RAB3A |
| | | interacting protein (rabin3)-like 1; talin 2; |
| | | similar to Ceruloplasmin precursor |
| | | (Ferroxidase); orosomucoid 1 precursor; |
| | | Orosomucoid-1 (alpha-1-acid |
| | | glycoprotein-1); Ig lambda chain precursor- |
| | | human; cold autoinflammatory syndrome |
| | | 1; chromosome 1 open reading frame 7; |
| | | angio-tensin/vasopres sin receptor; similar |
| | | to KIAA0913 protein; sodium channel, |
| | | voltage-gated, type V, alpha polypeptide; |
| | | hypothetical protein FLJ10379; |
| | | orosomucoid 2; alpha-1-acid glycoprotein, |
| | | type 2; Ig alpha-1 chain C region; |
| | | corticosteroid binding globulin precursor; |
| | | corticosteroid binding globulin; alpha-1 |
| | | anti-proteinase, antitrypsin; |
| | | KV3M_HUMAN IG KAPPA CHAIN V- |
| | | III REGION HIC PRECURSOR; |
| | | MUC_HUMAN Ig mu chain C region; |
| | | similar to Ig gamma-2 chain C region; |
| | | alpha-1-antichymotrypsin, precursor; |
| | | alpha-1-antichymotrypsin; |
| | | Antichymotrypsin; thyroid hormone |
| | | receptor-associated protein, 240 kDa |
| | | subunit; Ig heavy chain - human; Alpha-1- |
| | | antichymotrypsin precursor (ACT) |
| | | hypothetical protein XP_173158; |
| | | hypothetical protein DKFZp434G2226; |
| | | haptoglobin; Plasma protease C1 inhibitor |
| | | precursor (C1 Inh) (C1Inh) Haptoglobin-1 |
| | | precursor; leucine-rich alpha-2- |
| | | glycoprotein; S-arrestin; S-antigen; |
| | | NAD(P)H dehydrogenase, quinone 2; |
| | | NAD(P)H menadione oxidoreductase-1, |
| | | di-oxin-inducible-2; NAD(P)H menadione |
| | | oxi-doreductase 2, dioxin-inducible; |
| | | angiotensin precursor [validated] - human; |
| | | similar to KIAA1902 protein; similar to |
| | | KIAA1728 protein; calpain 3 isoform d; |
| | | calpain, large polypeptide L3; calpain |
| | | p94, large [catalytic] subunit; muscle- |
| | | specific calcium-activated neutral protease |
| | | 3 large subunit; asp (abnormal spindle)- |
| | | like, microcephaly associated; haptoglobin- |
| | | related protein; Haptoglobin-related locus; |
| | | Ig alpha-2 chain C region; hypothetical |
| | | protein DKFZp434P1818.1 - human |
| | | (fragment); GC3_HUMAN Ig gamma-3 |
| | | chain C region (Heavy chain disease |
| | | protein) (HDC) |
| Organ Rejection | miscellaneous | miR-658, miR-125a, miR-320, miR-381, |
| | | miR-628, miR-602, miR-629, or miR- |
| | | 125a; miR-324-3p, miR-611, miR-654, |
| | | miR-330_MM1, miR-524, miR-17- |
| | | 3p_MM1, miR-483, miR-663, miR-5,6-5p, |
| | | miR-326, miR-197 MM2, or miR-346; |
| | | matix metalloprotein-9, proteinase 3, or |
| | | HNP |
| Bone turnover/ | Urine | Pyridinoline, deoxypyridinoline, collagen |
| Osteoporosis | | type 1 corss-linked N-telopeptide (NTX), |
| | | collagen type 1 corss-linked C-telopeptide |
| | | (CTX), bone sialoprotein (BSP), Tartrate- |
| | | resistant acid phosphatase 5b |
| | saliva | deoxypyridinium (D-PYR) and osteocalcin |
| | | (OC), hepatocyte growth factor and |
| | | interleukin-1 beta |
| | Serum | Osteocalcin, alkaline phosphatase, bone- |
| | | specific alkaline phosphatase, serum type 1 |
| | | procollagen (C1NP, P1NP) |
| Jaw osteonecrosis | miscellaneous | PTH, insulin, TNF-α, leptin, OPN, OC, |
| | | OPG and IL6 |
| Gaucher's disease | (serum) | lyso-Gbl, Chitotriosidase and CCL18 |
| | urine | CCL18 |
| Traumatic brain injury | Miscellaneous | apoA-1, S-100B, isoprostane |
| | urine | GFAP, NGAL |
| | serum | neuron-specific enolase (NSE) |
| Septic shock | Miscellaneous | 15-Hydroxy-PG dehydrogenase (up), |
| | | LAIR1 (up), NFKB1A (up), TLR2, |
| | | PGLYPR1, TLR4, MD2, TLR5, IFNAR2, |
| | | IRAK2, IRAK3, IRAK4, PI3K, PI3KCB, |
| | | MAP2K6, MAPK14, NFKB1A, NFKB1, |
| | | IL1R1, MAP2K1IP1, MKNK1, FAS, |
| | | CASP4, GADD45B, SOCS3, TNFSF10, |
| | | TNFSF13B, OSM, HGF, or IL18R1 |
| Septic shock | Miscellaneous | IL-6, Protein-C, IL-1beta |
| Cancer | miscellaneous | FEN-1; CEA, NSE, CA 19-9, CA 125, |
| | | PSA, proGRP, SCC, NNMT, anti-p53 |
| | | autoantibodies, Separase and |
| | | DPPFV/Separase |
| | | SERPINA3; ACTB; AFM; AGT; AMBP; |
| | | APOF; AP0A2; APOC1; APOE; APOH; |
| | | SERPINC1; C1QB; C3; C4BPA; C8G; |
| | | C9; SERPINA6; CD14; CP; CRP; CSK; |
| | | F9; FGA; FGG; FLNA; FN1; GC; HRG; |
| | | IF; IGFALS; ITGA1; ITIH1; ITIH2; |
| | | ITIH4; KLKB1; LPA; MLL; MRC1; |
| | | MYL2; MYO6; ORM1; SERPINF1; |
| | | SERPINA1; SERPINA4; PROS1; QSCN6; |
| | | RGS4; SAA4; SERPINA7; TF; TFRC; |
| | | TTN; UBC; ALMS1; ATRN; PDCD11; |
| | | KIAA0433; SERPINA10; BCOR; |
| | | C10orf18; YY1AP1; FLJ10006; BDP1; |
| | | SMARCAD1; MKL2; CHST8; MCPH1; |
| | | MYO18B; MICAL-L1; PGLYRP2; |
| | | KCTD7; MGC27165; A1BG; A2M; |
| | | ABLIM1; ACTA1; AHSG; ANK3; APCS; |
| | | APOA1; APOA4; APOB; APOC3; |
| | | APOL1; AZGP1; B2M; BF; C1R; C1S; |
| | | C2; C4B; C5; C6; C7; C8A; C8B; |
| | | CDK5RAP2/CDK5RA2; CHGB; CLU; |
| | | COMP; CORO1A; CPN1; CUL1; DET1; |
| | | DSC1; F13A1; F2; F5; FGB; GOLGA1; |
| | | GSN; HBA1; HBB; HP; HPX; HSPA5; |
| | | HUNK; IGFBP5; IGHG1; IGLV4-3; |
| | | KIF5C; KNG1; KRT1; KRT10; KRT9; |
| | | LBP; LGALS3BP; LRG1; LUM; MMP14; |
| | | MYH4; NEB; NUCB2; ORM2; PF4V1; |
| | | PIGR; PLG; PON1; PPBP; RBP4; |
| | | RIMS1; RNF6; SAA1; SEMA3D; |
| | | SERPIND1; SERPINF2; SERPING1; |
| | | SF3B1; SPINK1; SPP1; SPTB; SYNE1; |
| | | TAF4B; TBC1D1; TLN1; TMSB4X; |
| | | TRIP11; TTR; UROC1; VTN; VWF; |
| | | ZFHX2; ZYX; |
| | | PSA (total prostate specific antigen), |
| | | Creatinine, Prostatic acid phosphatase, |
| | | PSA complexes, Prostrate-specific gene-1, |
| | | CA 12-5, Carcinoembryonic Antigen |
| | | (CEA), Alpha feto protein (AFP), hCG |
| | | (Human chorionic gonadotropin), Inhibin, |
| | | CAA Ovarian C1824, CA 27.29, CA 15-3, |
| | | CAA Breast C1924, Her-2, Pancreatic, CA |
| | | 19-9, CAA pancreatic, Neuron-specific |
| | | enolase, Angiostatin DcR3 (Soluble decoy |
| | | receptor 3), Endostatin, Ep-CAM (MK-1), |
| | | Free Immunoglobulin Light Chain Kappa, |
| | | Free Immunoglobulin Light Chain |
| | | Lambda, Herstatin, Chromogranin A, |
| | | Adrenomedullin, Integrin, Epidermal |
| | | growth factor receptor, Epidermal growth |
| | | factor receptor-Tyrosine kinase, Pro- |
| | | adrenomedullin N-terminal 20 peptide, |
| | | Vascular endothelial growth factor, |
| | | Vascular endothelial growth factor |
| | | receptor, Stem cell factor receptor, c- |
| | | kit/KDR, KDR, and Midkine; Zinc α2- |
| | | glycoprotein (ZAG) |
| Adenoma | miscellaneous | SI, DMBT1, CFI*, AQP1, APOD, |
| | | TNFRSF17, CXCL10, CTSE, IGHA1, |
| | | SLC9A3, SLC7A1, BATF2, SOCS1, |
| | | DOCK2, NOS2A, HK2, CXCL2, IL15RA, |
| | | POU2AF1, CLEC3B, ANI3BP, |
| | | MGC13057, LCK*, C4BPA, HOXC6, |
| | | GOLT1A, C2orf32, IL10RA, 240856_at, |
| | | SOCS3, MEIS3P1, HIPK1, GLS, CPLX1, |
| | | 236045_x_at, GALC, AMN, CCDC69, |
| | | CCL28, CPA3, TRIB2, HMGA2, PLCL2, |
| | | NR3C1, EIF5A, LARP4, RP5-1022P6.2, |
| | | PHLDB2, FKBP1B, INDO, CLDN8, |
| | | CNTN3, PBEF1, SLC16A9, CDC25B, |
| | | TPSB2, PBEF1, ID4, GJB5, CHN2, |
| | | LIMCH1, or CXCL9; ABCA8, |
| | | KIAA1199, GCG, MAMDC2, C2orf32, |
| | | 229670_at, IGF1, PCDH7, PRDX6, |
| | | PCNA, COX2, or MUC6 |
| Head and Neck cancer | saliva | IL-1, IL-6, IL-8, VEGF, MMP-9, TGF-β, |
| | | TNF-α, MMP-7, plasminogen activated |
| | | (PA), uPA, IGF, or INF-2 |
| Barrett's esophagus | miscellaneous | miR-21, miR-143, miR-145, miR-194, or |
| | | miR-215; S100A2, S100A4; p53, MUC1, |
| | | MUC2 |
| Lung cancer | miscellaneous | miR-21, miR-205, miR-221 (protective), |
| | | let-7a (protective), miR-137 (risky), miR- |
| | | 372 (risky), or miR-122a (risky); miR-17- |
| | | 92, miR-19a, miR-92, miR-155, miR-191, |
| | | or miR-210; EGFR, PTEN, RRM1, RRM2, |
| | | ABCB1, ABCG2, LRP, VEGFR2, |
| | | VEGFR3, class III b-tubulin; KRAS, |
| | | hENT1; RLF-MYCL1, TGF-ALK, or |
| | | CD74-ROS1 |
| | saliva | CCNI, EGFR, FGF19, FRS2, and GREB1 |
| | | LZTS, BRAF, FRS2, ANXA1, |
| | | Haptoglobin Hp2, Zinc Alpha2- |
| | | Glycoprotein, Calprotectin, |
| | | Porphyromonas catoniae 16S rRNA, |
| | | Campylobacter showae 16S rRNA, |
| | | Streptocococcus salivaris 16S rRNA, |
| | | Campylobacter rectus 16S rRNA, |
| | | Veillonella parvula 16S rRNA, Kigella |
| | | oralis 16S rRNA, and Granulicatella |
| | | adiacens 16S rRNA |
| Pancreatic cancer | miscellaneous | miR-221, miR-181 a, miR-155, miR-210, |
| | | miR-213, miR-181b, miR-222, miR-181b- |
| | | 2, miR-21, miR-181b-1, miR-220, miR- |
| | | 181d, miR-223, miR-100-1/2, miR-125a, |
| | | miR-143, miR-10a, miR-146, miR-99, |
| | | miR-100, miR-199a-1, miR-10b, miR- |
| | | 199a-2, miR-221, miR-181a, miR-155, |
| | | miR-210, miR-213, miR-181b, miR-222, |
| | | miR-181b-2, miR-21, miR-181b-1, miR- |
| | | 181c, miR-220, miR-181d, miR-223, miR- |
| | | 100-1/2, miR-125a, miR-143, miR-10a, |
| | | miR-146, miR-99, miR-100, miR-199a-1, |
| | | miR-10b, miR-199a-2, miR-107, miR-103, |
| | | miR-103-2, miR-125b-1, miR-205, miR- |
| | | 23a, miR-221, miR-424, miR-301, miR- |
| | | 100, miR-376a, miR-125b-1, miR-21, |
| | | miR-16-1, miR-181a, miR-181c, miR-92, |
| | | miR-15, miR-155, let-7f-1, miR-212, miR- |
| | | 107, miR-024-1/2, miR-18a, miR-31, miR- |
| | | 93, miR-224, or let-7d; miR-148a, miR- |
| | | 148b, miR-375, miR-345, miR-142, miR- |
| | | 133a, miR-216, miR-217 or miR-139; |
| | | KRAS, CTNNLB1, AKT, NCOA3, or B- |
| | | RAF; BRCA2, PALB2, or p16 |
| | saliva | MBD3L2, KRAS, STIM2, DMXL2, |
| | | ACRV1, DMD and CABLES1, TK2, |
| | | GLTSCR2, CDKL3, TPT1 and DPM1 |
| Breast cancer | miscellaneous | miR-21, miR-155, miR-206, miR-122a, |
| | | miR-210, miR-155, miR-206, miR-210, or |
| | | miR-21; let-7, miR-10b, miR-125a, miR- |
| | | 125b, miR-145, miR-143, miR-16, miR- |
| | | 10b, miR-125a; hsp70, MART-1, TRP, |
| | | HER2, hsp70, MART-1, TRP, HER2, ER, |
| | | PR, Class III b-tubulin, or VEGFA; GAS5; |
| | | ETV6-NTRK3; |
| | Saliva | CAH6 (Carbonic anhydrase VI), K2C4 |
| | | (Cytokeratin 4), CYTA (Cystatin A), |
| | | FABP4 (Epid. Fatty acid binding prot.), |
| | | IGHGI (Ig gamma-1 chain C region), |
| | | TRFL (Lactoferrin), BPIL1 (Bact. Perm.- |
| | | increasing prot.-1), CYTC (Cystatin C), |
| | | HPT (Haptoglobin), PROF1 (Profilin-1), |
| | | ZA2G (Zinc-alpha-2-glycoprotein), ENOA |
| | | (Alpha enolase), IGHA2 (Ig alpha-2 chain |
| | | C region), IL-1 ra (Interleukin-1 receptor |
| | | anatagonist protein precursor), S10A7 |
| | | (S100 calcium-binding protein A7), and |
| | | SPLC2 (Short palate, lung and nasel epith |
| | | Carc. assoc. protein 2) |
| Ovarian cancer | Saliva | c-erbB-2, cancer antigen 15-3, p53 |
| | urine | HER2/neu (c-erbB-2) |
| | | 47D10 antigen, PTCD2, SLC25A20, |
| | | NFKB2, RASGRP2, PDE7A, MLL, |
| | | PRKCE, GPATC3, PRIC285 and GSTA4 |
| | | MIPEP, PLCB2, SLC25A19, DEF6, |
| | | ZNF236, C18orf22, COX7A2, DDX11, |
| | | TOP3A, C9orf6, UFC1, PFDN2, KLRD1, |
| | | LOC643641, HSP90AB1, CLCN7, |
| | | TNFAIP2, PRKCE, MRPL40, FBF1, |
| | | ANKRD44, CCT5, USP40, UBXD4, |
| | | LRCH1, MRPL4, SCCPDH, STX6, |
| | | LOC284184, FLJ23235, GPATC3, CPSF4, |
| | | CREM, HIST1H1D, HPS4, FN3KRP, |
| | | ANKRD16, C8 orf16, ATF71P2, PRIC285 |
| | Miscellaneous | miR-200a, miR-141, miR-200c, miR-200b, |
| | | miR-21, miR-200a, miR-200b, miR-200c, |
| | | miR-203, miR-205, miR-214, miR-199″, or |
| | | miR-215; miR-199a, miR-140, miR-145, |
| | | miR-100, miR-let-7 cluster, or miR-125b- |
| | | 1; ERCC1, ER, TOPO1, TOP2A, AR, |
| | | PTEN, HER2/neu, CD24 or EGFR; |
| | | VEGFA, VEGFR2, or HER2 |
| Ovarian cancer | Saliva | CA 125 |
| Prostate cancer | Saliva | AGPAT1, B2M, BASP2, IER3, and IL1B |
| | Miscellaneous | miR-9, miR-21, miR-141, miR-370, miR- |
| | | 200b, miR-210, miR-155, or miR-196a; |
| | | miR-202, miR-210, miR-296, miR-320, |
| | | miR-370, miR-373, miR-498, miR-503, |
| | | miR-184, miR-198, miR-302c, miR-345, |
| | | miR-491, miR-513, miR-32, miR-182, |
| | | miR-31, miR-26a-1/2, miR-200c, miR-375, |
| | | miR-196a-1/2, miR-370, miR-425, miR- |
| | | 425, miR-194-1/2, miR-181a-1/2, miR- |
| | | 34b, let-71, miR-188, miR-25, miR-106b, |
| | | miR-449, miR-99b, miR-93, miR-92-1/2, |
| | | miR-125a, or miR-141; let-7a, let-7b, let- |
| | | 7c, let-7d, let-7g, miR-16, miR-23a, miR- |
| | | 23b, miR-26a, miR-92, miR-99a, miR-103, |
| | | miR-125a, miR-125b, miR-143, miR-145, |
| | | miR-195, miR-199, miR-221, miR-222, |
| | | miR-497, let-7f, miR-19b, miR-22, miR- |
| | | 26b, miR-27a, miR-27b, miR-29a, miR- |
| | | 29b, miR-30_5p, miR-30c, miR-100, miR- |
| | | 141, miR-148a, miR-205, miR-520h, miR- |
| | | 494, miR-490, miR-133a-1, miR-1-2, miR- |
| | | 218-2, miR-220, miR-128a, miR-221, |
| | | miR-499, miR-329, miR-340, miR-345, |
| | | miR-410, miR-126, miR-205, miR-7-1/2, |
| | | miR-145, miR-34a, miR-487, or let-7b; |
| | | miR-15a, miR-16-1, miR-143 or miR-145; |
| | | AR, PCA3; FASLG or TNFSF10; U50; |
| | | ACSL3-ETV1, C150RF21-ETV1, |
| | | FLJ35294-ETV1, HERV-ETV1, |
| | | TMPRSS2-ERG, TMPRSS2-ETV1/4/5, |
| | | TMPRSS2-ETV4/5, SLC5A3-ERG, |
| | | SLC5A3-ETV1, SLC5A3-ETV5 or KLK2- |
| | | ETV4 |
| | | kallikrein-2 (KLK2), C reactive protein |
| | | (CRP), cysteine-rich secretory protein 3 |
| | | (CRISP3) and chromogranin A (CHGA), |
| | | comprises prostatic acid phosphatase |
| | | (PAP), lactate dehydrogenase (LDH), |
| | | alkaline phosphatase (ALP) |
| | saliva | PSA |
| Esophageal Cancer | urine | PCA3, GOLPH2, SPINK1, |
| | | TMPRSS2:ERG |
| | miscellaneous | miR-192, miR-194, miR-21, miR-200c, |
| | | miR-93, miR-342, miR-152, miR-93, miR- |
| | | 25, miR-424, or miR-151; miR-27b, miR- |
| | | 205, miR-203, miR-342, let-7c, miR-125b, |
| | | miR-100, miR-152, miR-192, miR-194, |
| | | miR-27b, miR-205, miR-203, miR-200c, |
| | | miR-99a, miR-29c, miR-140, miR-103, or |
| | | miR-107; |
| Gastric cancer | miscellaneous | miR-106a, miR-21, miR-191, miR-223, |
| | | miR-24-1, miR-24-2, miR-107, miR-92-2, |
| | | miR-214, miR-25, or miR-221; let-7a; |
| | | RRM2, or surviving; EphA4 |
| Gastrointestinal | miscellaneous | DOG-1, PKC-theta, KIT, GPR20, PRKCQ, |
| Stromal Tumor (GIST) | | KCNK3, KCNH2, SCG2, TNFRSF6B, or |
| | | CD34; PDGFRA, c-kit |
| Colorectal carcinoma | miscellaneous | miR-24-1, miR-29b-2, miR-20a, miR-10a, |
| | | miR-32, miR-203, miR-106a, miR-17-5p, |
| | | miR-30c, miR-223, miR-126, miR-128b, |
| | | miR-21, miR-24-2, miR-99b, miR-155, |
| | | miR-213, miR-150, miR-107, miR-191, |
| | | miR-221, miR-20a, miR-510, miR-92, |
| | | miR-513, miR-19a, miR-21, miR-20, miR- |
| | | 183, miR-96, miR-135b, miR-31, miR-21, |
| | | miR-92, miR-222, miR-181b, miR-210, |
| | | miR-20a, miR-106a, miR-93, miR-335, |
| | | miR-338, miR-133b, miR-346, miR-106b, |
| | | miR-153a, miR-219, miR-34a, miR-99b, |
| | | miR-185, miR-223, miR-211, miR-135a, |
| | | miR-127, miR-203, miR-212, miR-95, or |
| | | miR-17-5p; miR-143, miR-145, miR-143, |
| | | miR-126, miR-34b, miR-34c, let-7, miR-9- |
| | | 3, miR-34a, miR-145, miR-455, miR-484, |
| | | miR-101, miR-145, miR-133b, miR-129, |
| | | miR-124a, miR-30-3p, miR-328, miR- |
| | | 106a, miR-17-5p, miR-342, miR-192, |
| | | miR-1, miR-34b, miR-215, miR-192, miR- |
| | | 301, miR-324-5p, miR-30a-3p, miR-34c, |
| | | miR-331, or miR-148b; EFNB1, ERCC1, |
| | | HER2, VEGF, or EGFR; AFRs, Rabs, |
| | | ADAM10, CD44, NG2, ephrin-B1, MIF, |
| | | b-catenin, Junction, plakoglobin, glalectin- |
| | | 4, RACK1, tetrspanin-8, FasL, TRAIL, |
| | | A33, CEA, EGFR, dipeptidase 1, hsc-70, |
| | | tetraspanins, ESCRT, TS, PTEN, or |
| | | TOPO1; GREM1, DDR2, GUCY1A3, |
| | | TNS1, ADAMTS1, FBLN1, FLJ38028, |
| | | RDX, FAM129A, ASPN, FRMD6, MCC, |
| | | RBMS1, SNA12, MEIS1, DOCK10, |
| | | PLEKHC1, FAM126A, TBC1D9, VWF, |
| | | DCN, ROBO1, MSRB3, LATS2, MEF2C, |
| | | IGFBP3, GNB4, RCN3, AKAP12, |
| | | RFTN1, 226834_at, COL5A1, GNG2, |
| | | NR3C1*, SPARCL1, MAB21L2, AXIN2, |
| | | 236894_at, AEBP1, AP1S2, C10orf56, |
| | | LPHN2, AKT3, FRMD6, COL15A1, |
| | | CRYAB, COL14A1, LOC286167, QKI, |
| | | WWTR1, GNG11, PAPPA, or ELDT1; |
| | | 227458_at, INDO, CXCL9, CCR2, CD38, |
| | | RARRES3, CXCL10, FAM26F, TNIP3, |
| | | NOS2A, CCRL1, TLR8, IL18BP, FCRL5, |
| | | SAMD9L, ECGF1, TNFSF13B, GBPS, or |
| | | GBP1; TMEM37*, IL33, CA4, CCDC58, |
| | | CLIC6, VERSUSNL1, ESPN, APCDD1, |
| | | C13orf18, CYP4X1, ATP2A3, |
| | | LOC646627, MUPCDH, ANPEP, |
| | | C1orf115, HSD3B2, GBA3, GABRB2, |
| | | GYLTL1B, LYZ, SPC25, CDKN2B, |
| | | FAM89A, MOGAT2, SEMA6D, |
| | | 229376_at, TSPAN5, IL6R, or SLC26A2 |
| Melanoma | miscellaneous | miR-19a, miR-144, miR-200c, miR-211, |
| | | miR-324-5p, miR-331, or miR-374; miR-9, |
| | | miR-15a, miR-17-3p, miR-23b, miR-27a, |
| | | miR-28, miR-29b, miR-30b, miR-31, miR- |
| | | 34b, miR-34c, miR-95, miR-96, miR-100, |
| | | miR-104, miR-105, miR-106a, miR-107, |
| | | miR-122a, miR-124a, miR-125b, miR-127, |
| | | miR-128a, miR-128b, miR-129, miR-135a, |
| | | miR-135b, miR-137, miR-138, miR-139, |
| | | miR-140, miR-141, miR-149, miR-154, |
| | | miR-154#3, miR-181a, miR-182, miR-183, |
| | | miR-184, miR-185, miR-189, miR-190, |
| | | miR-199, miR-199b, miR-200a, miR-200b, |
| | | miR-204, miR-213, miR-215, miR-216, |
| | | miR-219, miR-222, miR-224, miR-299, |
| | | miR-302a, miR-302b, miR-302c, miR- |
| | | 302d, miR-323, miR-325, let-7a, let-7b, |
| | | let-7d, let-7e, or let-7g; MUM-1, beta- |
| | | catenin, or Nop/5/Sik; DUSP-1, Alix, |
| | | hsp70, Gib2, Gia, moesin, GAPDH, malate |
| | | dehydrogenase, p120 catenin, PGRL, |
| | | syntaxin-binding protein 1 & 2, septin-2, or |
| | | WD-repeat containing protein 1; H/ACA |
| | | (U1071), SNORA11D |
| Head and neck cancer | miscellaneous | miR-21, let-7, miR-18, miR-29c, miR-142- |
| | | 3p, miR-155, miR-146b, miR-205, or miR- |
| | | 21; miR-494; HPV E6, HPV E7, p53, IL-8, |
| | | SAT, H3FA3; EGFR, EphB4, or EphB2; |
| | | CHCHD7-PLAG1, CTNNB1-PLAG1, |
| | | FHIT-HMGA2, HMGA2-NFIB, LIFR- |
| | | PLAG1, or TCEA1-PLAG1 |
| Oral squamous cell | saliva | p53 autoantibodies, defensing-1, lncRNAs |
| carcinoma | | (MEG-3, MALAT-1, HOTAIR, NEAT-1, |
| | | UCA) Cortisol, lactate dehydrogenase, |
| | | Transferrin, cyclin D1, Maspin, alpha- |
| | | amylase, IL-8, TNF-α, IL-1, IL-6, Basic |
| | | fibroblast growth factor, Statherin, Cyfra |
| | | 21.1, TPA, CA125, Endothelin-1, IL-1β, |
| | | CD44, IGF-1, MMP-2, MMP-9, CD59, |
| | | Catalase, Profilin, S100A9/MRP14, M2BP, |
| | | CEA, Carcinoma associated antigen |
| | | CA-50, Salivary carbonyls, Maspin, |
| | | 8-oxoguanine DNA glycosylase, OGG1, |
| | | Phosphorylated-Src, Ki-67, Zinc finger |
| | | protein 501 peptide, Hemopexin, |
| | | Haptoglobin, Complement C3, |
| | | Transthyretin, α1-antitrypsin, Peroxidase, |
| | | GST, SOD, 8-OHdG, Glutathione, MDA, |
| | | miR-125a, miR-200a, miR-31 |
| Salivary gland tumors | miscellaneous | Fibroblast growth factor 2 (FGF2) and |
| | | fibroblast growth factor receptor 1 |
| | | (FGFR1) |
| Hepatocellular | miscellaneous | miR-221; et-7a-1, let-7a-2, let-7a-3, let-7b, |
| carcinoma | | let-7c, let-7d, let-7e, let-7f-2, let-fg, miR- |
| | | 122a, miR-124a-2, miR-130a, miR-132, |
| | | miR-136, miR-141, miR-142, miR-143, |
| | | miR-145, miR-146, miR-150, miR- |
| | | 155(BIC), miR-181a-1, miR-181a-2, miR- |
| | | 181c, miR-195, miR-199a-1-5p, miR- |
| | | 199a-2-5p, miR-199b, miR-200b, miR- |
| | | 214, miR-223, or pre-miR-594; miR-122, |
| | | miR-100, or miR-10a; miR-198 or miR- |
| | | 145 |
| Renal cell carcinoma | miscellaneous | miR-141, miR-200; miR-28, miR-185, |
| | | miR-27, miR-let-7f-2; laminin receptor 1, |
| | | betaig-h3, Galectin-1, a-2 Macroglobulin, |
| | | Adipophilin, Angiopoietin 2, Caldesmon 1, |
| | | Class II MHC-associated invariant chain |
| | | (CD74), Collagen IV-al, Complement |
| | | component, Complement component 3, |
| | | Cytochrome P450, subfamily IIJ |
| | | polypeptide 2, Delta sleep-inducing |
| | | peptide, Fc g receptor 111a (CD16), HLA- |
| | | B, HLA-DRa, HLA-DRb, HLA-SB, IFN- |
| | | induced transmembrane protein 3, IFN- |
| | | induced transmembrane protein 1, or Lysyl |
| | | Oxidase; IF1 alpha, VEGF, PDGFRA; |
| | | ALPHA-TFEB, NONO-TFE3, PRCC- |
| | | TFE3, SFPQ-TFE3, CLTC-TFE3, or |
| | | MALAT1-TFEBf |
| Renal cell carcinoma | miscellaneous | Akt, total Erk1/2, total Met, total GSK3b, |
| | | total Hif1a, total p21, total AMPKa1, total |
| | | VEGF, total PlGF, total VEGFR-1/Flt-1, |
| | | phosphorylated Akt, phosphorylated |
| | | Erk1/2, phosphorylated. Met, |
| | | phosphorylated STAT3, phosphorylated |
| | | GSK3b, and phosphorylated AMPKa1 |
| Cervical cancer | miscellaneous | HPV E6, HPV E7, or p53 |
| Thyroid cancer | miscellaneous | AKAP-BRAF, CCDC6-RET, ERC1- |
| | | RETM, GOLGA5-RET, HOOK3-RET, |
| | | HRH4-RET, KTN1-RET, NCOA4-RET, |
| | | PCM1-RET, PRKARA1A-RET, RFG- |
| | | RET, RFG9-RET, Ria-RET, TGF-NTRK1, |
| | | TPM3-NTRK1, TPM3-TPR, TPR-MET, |
| | | TPR-NTRK1, TRIM24-RET, TRIM27- |
| | | RET or TRIM33-RET; PAX8-PPARy |
| Neuroblastoma | Urine | Neuron-specific enolase (NSE) |
| Glioblastoma | serum | GFAP |
| Brain cancer | miscellaneous | miR-21, miR-10b, miR-130a, miR-221, |
| | | miR-125b-1, miR-125b-2, miR-9-2, miR- |
| | | 21, miR-25, or miR-123; miR-128a, miR- |
| | | 181c, miR-181a, or miR-181b; GOPC- |
| | | ROS1; MGMT; EGFR |
| Blood Cancers | miscellaneous | HOX11, TAL1, LY1, LMO1, or LMO2; |
| | | TTL-ETV6, CDK6-MLL, CDK6-TLX3, |
| | | ETV6-FLT3, ETV6-RUNX1, ETV6-TTL, |
| | | MLL-AFF1, MLL-AFF3, MLL-AFF4, |
| | | MLL-GAS7, TCBA1-ETV6, TCF3-PBX1 |
| | | or TCF3-TFPT, for acute lymphocytic |
| | | leukemia (ALL); BCL11B-TLX3, IL2- |
| | | TNFRFS17, NUP214-ABL1, NUP98- |
| | | CCDC28A, TAL1-STIL, or ETV6-ABL2, |
| | | for T-cell acute lymphocytic leukemia (T- |
| | | ALL); ATIC-ALK, KIAA1618-ALK, |
| | | MSN-ALK, MYH9-ALK, NPM1-ALK, |
| | | TGF-ALK or TPM3-ALK, for anaplastic |
| | | large cell lymphoma (ALCL); BCR-ABL1, |
| | | BCR-JAK2, ETV6-EVI1, ETV6-MN1 or |
| | | ETV6-TCBA1, for chronic myelogenous |
| | | leukemia (CML); CBFB-MYH11, CHIC2- |
| | | ETV6, ETV6-ABL1, ETV6-ABL2, ETV6- |
| | | ARNT, ETV6-CDX2, ETV6-HLXB9, |
| | | ETV6-PER1, MEF2D-DAZAP1, AML- |
| | | AFF1, MLL-ARHGAP26, MLL- |
| | | ARHGEF12, MLL-CASC5, MLL-CBL, |
| | | MLL-CREBBP, MLL-DAB21P, MLL- |
| | | ELL, MLL-EP300, MLL-EPS15, MLL- |
| | | FNBP1, MLL-FOXO3A, MLL-GMPS, |
| | | MLL-GPHN, MLL-MLLT1, MLL- |
| | | MLLT11, MLL-MLLT3, MLL-MLLT6, |
| | | MLL-MYO1F, MLL-PICALM, MLL- |
| | | SEPT2, MLL-SEPT6, MLL-SORBS2, |
| | | MYST3-SORBS2, MYST-CREBBP, |
| | | NPM1-MLF1, NUP98-HOXA13, |
| | | PRDM16-EVI1, RABEP1-PDGFRB, |
| | | RUNX1-EVI1, RUNX1-MDS1, RUNX1- |
| | | RPL22, RUNX1-RUNX1T1, RUNX1- |
| | | SH3D19, RUNX1-USP42, RUNX1- |
| | | YTHDF2, RUNX1-ZNF687, or TAF15- |
| | | ZNF-384, for AML; CCND1-FSTL3, for |
| | | chronic lymphocytic leukemia (CLL); and |
| | | FLIP1-PDGFRA, FLT3-ETV6, |
| | | KIAA1509-PDGFRA, PDE4DIP- |
| | | PDGFRB, NIN-PDGFRB, TP53BP1- |
| | | PDGFRB, or TPM3-PDGFRB, for hyper |
| | | eosinophilia/chronic eosinophilia; miR- |
| | | 23b, miR-24-1, miR-146, miR-155, miR- |
| | | 195, miR-221, miR-331, miR-29a, miR- |
| | | 195, miR-34a, or miR-29c; miR-15a, miR- |
| | | 16-1, miR-29 or miR-223; miR-128b, miR- |
| | | 204, miR-218, miR-331, miR-181b-1, |
| | | miR-17-92 |
| B-Cell Chronic | miscellaneous | miR-183-prec, miR-190, miR-24-1-prec, |
| Lymphocytic | | miR-33, miR-19a, miR-140, miR-123, |
| Leukemia | | miR-10b, miR-15b-prec, miR-92-1, miR- |
| | | 188, miR-154, miR-217, miR-101, miR- |
| | | 141-prec, miR-153-prec, miR-196-2, miR- |
| | | 134, miR-141, miR-132, miR-192, or miR- |
| | | 181b-prec; miR-213, miR-220; ZAP70, |
| | | AdipoR1; BCL3-MYC, MYC-BTG1, |
| | | BCL7A-MYC, BRWD3-ARHGAP20 or |
| | | BTG1-MYC |
| B-cell lymphoma | miscellaneous | miR-17-92 polycistron, miR-155, miR- |
| | | 210, or miR-21, miR-19a, miR-92, miR- |
| | | 142 miR-155, miR-221 miR-17-92, miR- |
| | | 21, miR-191, miR-205, U50; miR-17-92, |
| | | miR-155, miR-210, or miR-21; A-myb, |
| | | LMO2, JNK3, CD10, bcl-6, Cyclin D2, |
| | | IRF4, Flip, or CD44; CITTA-BCL6, |
| | | CLTC-ALK, IL21R-BCL6, PIM1-BCL6, |
| | | TFCR-BCL6, IKZF1-BCL6 or SEC31A- |
| | | ALK |
| Burkitt's lymphoma | miscellaneous | pri-miR-155; MYC, TERT, NS, NP, MAZ, |
| | | RCF3, BYSL, IDE3, CDC7, TCL1A, |
| | | AUTS2, MYBL1, BMP7, ITPR3, CDC2, |
| | | BACK2, TTK, MME, ALOX5, or TOP1; |
| | | BCL6, KI-67; IGH-MYC, LCP1-BCL6 |
| Endometrial cancer | miscellaneous | miR-185, miR-106a, miR-181a, miR-210, |
| | | miR-423, miR-103, miR-107, or let-7c; |
| | | miR-71, miR-221, miR-193, miR-152, or |
| | | miR-30c; NLRP7, AlphaV Beta6 integrin |
| uterine leiomyomas | miscellaneous | let-7 family member, miR-21, miR-23b, |
| | | miR-29b, or miR-197 |
| myelofibrosis | miscellaneous | miR-190; miR-31, miR-150 and miR-95; |
| | | miR-34a, miR-342, miR-326, miR-105, |
| | | miR-149, miR-147 |
| Pheochromocytoma | Urine | Catecholamines (epinephrine, |
| | | norepinephrine, adrenaline) |
| Kidney disease/injury | miscellaneous | ADBP-26, NHE3, KIM-1, |
| | | glutamyltransferase, N-acetyl-beta-D- |
| | | glucosaminidase, lysozyme, NGAL, L- |
| | | FABP, bikunin, urea, prostaglandins, |
| | | creatinine, alpha-1-microglobulin, retinol |
| | | binding protein, glutathione-S-transferases, |
| | | adiponectin, beta-2-macroglobuin, |
| | | calbindin-D, cysteine-rich angiogenic |
| | | inducer 61, endothelial/epithial growth |
| | | factors, alpha-1-acid glycoprotein |
| | | (orosomucoid), prealbumin, modified |
| | | albumin, albumin, transferrin, alpha-1- |
| | | lipoprotein, alpha-1-antitrypsin matrix |
| | | metalloproteinases (MMPs), alpha-1- |
| | | fetoprotein, Tamm Horsfall protein, |
| | | homoarginine, interleukin 18, monocyte |
| | | chemotactic protein-1 (MCP-1), Lipocalin, |
| | | VCAN, NRP1, CCL2, CCL19, COL3A1, |
| | | GZMM, alpha-galactosidase, casein kinase |
| | | 2, IP-10, Mig, I-TAC, MIP-1α, MIP-3α, |
| | | and MIP-1β, alpha-2-glycoprotein-Zinc, |
| | | leucine-rich alpha-2-glycoprotein, |
| | | uromodulin, Pacsin 2, hepcidin-20, |
| | | hepcidin-25, AIF-2, urinary type-IV |
| | | collagen, lipocalin-type prostaglandin D |
| | | synthase (L-PGDS), urinary neutrophil |
| | | gelatinase-associated lipocalin (uNGAL), |
| | | Annexin A1, Rab23, Shh, Ihh, Dhh, |
| | | PTCH1, PTCH2, SMO, Gli1, Gli2, Gli3, |
| | | TLR4, cystatin C, AQP1, AQP2, AQP3, |
| | | NKCC2, NaPill, DAHKSEVAHRFKD |
| | | [RNA:] SLC12A1, UMOD, vWF, MMP1, |
| | | MMP3, SLC22A6, SLC22A 8, SLC22A |
| | | 12, podocin, cubulin, LRP2, AQP9, and |
| | | albumin, carcinoembryonic antigen (CEA), |
| | | mucin, alpha-fetoprotein, tyrosinase, |
| | | melanoma associated antigen, mutated |
| | | tumor protein 53, p21, PUMA, prostate- |
| | | specific antigen (PSA) or thyroglobulin, |
| | | von Willebrand factor (VWF), thrombin, |
| | | factor VIII, plasmin, fibrin, osteopontin |
| | | (SPP1), Rab23, Shh, Ihh, Dhh, PTCH1, |
| | | PTCH2, SMO, Gli1, Gli2, Gli3 |
| | urine | L-FABP, NGAL |
| Liver failure/disease | saliva | Lactoferrin, uric acid, cortisol, alpha- |
| | | amylase |
| | miscellaneous | Carnitine; Cholic Acid; Chenodeoxycholic, |
| | | Deoxycholic, Lithocholic, Glycocholic; |
| | | Prostaglandin E₂; 13,14-dihydro-15-keto |
| | | Prostaglandin A2; Prostaglandin B2; |
| | | Prostaglandin F2a; 15-keto-Prostaglandin |
| | | F2α; 6-keto-Prostaglandin F1α; |
| | | Thromboxane B2; 11-dehydro- |
| | | Thromboxane B2; Prostaglandin D2; |
| | | Prostaglandin J2; |
| | | 15-deoxy-Δ12,14-Prostaglandin J2; 11β- |
| | | Prostaglandin F2α; 5(S)- |
| | | Hydroxyeicosatetraenoic acid; 5(S)- |
| | | Hydroxyeicosapentaenoic acid; |
| | | Leukotriene B4; Leukotriene B5; |
| | | Leukotriene C4; Leukotriene D4; |
| | | Leukotriene E4; Leukotriene F4; 12(S)- |
| | | Hydroxyeicosatetraenoic acid; 12(S)- |
| | | Hydroxyeicosapentaenoic acid; 15(S)- |
| | | Hydroxyeicosatetraenoic acid; 15(S)- |
| | | Hydroxyeicosapentaenoic acid; Lipoxin |
| | | A4; 8(S)-Hydroxyeicosatetraenoic acid; 9- |
| | | Hydroxyeicosatetraenoic acid; 11- |
| | | Hydroxyeicosatetraenoic acid; 8-iso- |
| | | Prostaglandin F2α; 9- |
| | | Hydroxyoctadecadienoic acid; 13- |
| | | Hydroxyoctadecadienoic acid; 20(S)- |
| | | Hydroxyeicosatetraenoic acid; 9,10- |
| | | Epoxyoctadecenoic acid; 12,13- |
| | | Epoxyoctadecenoic acid; 12,13- |
| | | Dihydroxyoctadecenoic acid; 5,6- |
| | | Epoxyeicosatrienoic acid; 11,12- |
| | | Epoxyeicosatrienoic acid; 14,15- |
| | | Epoxyeicosatrienoic acid; 5,6- |
| | | Dihydroxyeicosatrienoic acid; 8,9- |
| | | Dihydroxyeicosatrienoic acid; 11,12- |
| | | Dihydroxyeicosatrienoic acid; 14,15- |
| | | Dihydroxyeicosatrienoic acid; 14,15- |
| | | Epoxyeicosatetraenoic acid; 17,18- |
| | | Epoxyeicosatetraenoic acid; 14,15- |
| | | Dihydroxyeicosatetraenoic acid; 17,18- |
| | | Dihydroxyeicosatetraenoic acid; 19,20- |
| | | Dihydroxydocosapentaenoic acid; |
| | | diacetylspermine, hemopexin, TLR4 |
| Stroke | miscellaneous | MMP9, S100-P, S100A12, SI00A9, coag |
| | | factor V, ArginaseI, CA-IV, |
| | | monocarboxylic acid transporter, ets-2, |
| | | EIF2alpha, cytoskeleton associated protein |
| | | 4, N-formylpeptide receptor, |
| | | Ribonuclease2, N-acetylneuraminate |
| | | pyruvate lyase, BCL-6, or Glycogen |
| | | phosphorylase |
| Heart failure/ | urine | 8-iso-prostaglandin F2α (8-iso-PGF2α) |
| Cardiovascular health | miscellaneous | miR-195, miR-208, miR-214, let-7b, let- |
| | | 7c, let-7e, miR-15b, miR-23a, miR-24, |
| | | miR-27a, miR-27b, miR-93, miR-99b, |
| | | miR-100, miR-103, miR-125b, miR-140, |
| | | miR-145, miR-181a, miR-191, miR-195, |
| | | miR-199a, miR-320, miR-342, miR-451, |
| | | or miR-499; miR-1, miR-10a, miR-17-5p, |
| | | miR-19a, miR-19b, miR-20a, miR-20b, |
| | | miR-26b, miR-28, miR-30e-5p, miR-101, |
| | | miR-106a, miR-126, miR-222, miR-374, |
| | | miR-422b, or miR-423; MRP14, CD69; |
| | | CK-MB, cTnI (cardiac troponin), CRP, |
| | | BPN, IL-6, MCSF, CD40, CD40L |
| | miscellaneous | SFRP-3, NT-proBNP, troponin T, |
| | saliva | SKITHRIHWESASLL, AHKSEVAHRFK, |
| | | uroguanylin, BNP |
| | | miR-378, miR-497, miR-21, miR-15b, |
| | | miR-99a, miR 29a, miR-24, miR-30b, |
| | | miR-29c, miR-331.3p, miR-19a, miR-22, |
| | | miR-126, let-7b, miR-502.3, and miR-652; |
| | | IL-16, sFas, Fas ligand, MCP-3, HGF, |
| | | CTACK, EOTAXIN, adiponectin, IL-18, |
| | | TIMP.4, TIMP.1, CRP, VEGF, and EGF |
| | | C-reactive protein (CRP); myoglobin |
| | | (MYO), creatinine kinase myocardial band |
| | | (CK-MB), cardiac troponins (cTn), and |
| | | myeloperoxidase; TNF-α, and MMP-9; |
| | | CD40 |
| Vulnerable plaque | saliva | Amylase |
| | miscellaneous | L-6, MMP-9, PAPP-A, D-dimer, |
| | | fibrinogen, Lp-PLA2, SCD40L, Il-18, |
| | | oxLDL, GPx-1, MCP-1, P1GF, or CRP |
| High blood pressure | saliva | lysozyme |
| Fibromyalgia | miscellaneous | NR2D |
| Neuropathic Pain | miscellaneous | CCR2/4, CNP; ICAM-1, CGRP, TIMP-1, |
| | | CLR-1, HSP-27, FABP, or apolipoprotein |
| | | D; OX42, ED9 |
| Tiredness/fatigue | saliva | PPGKPQGPPPQGGNQPQGPPPPPGKPQ |
| | | (SEQ ID NO: 1); |
| | | GNPQGPSPQGGNKPQGPPPPPGKPQ |
| | | (SEQ ID NO: 2); |
| | | SPPGKPQGPPQQEGNKPQGPPPPGKPQ |
| | | (SEQ ID NO: 3) |
| | urine | endorepellin |
| | | human herpesvirus 6, human herpesvirus 7, |
| | | human cytomegalovirus, and Epstein-Barr |
| | | virus (EBV) |
| | miscellaneous | GGHPPPP (SEQ ID NO: 4), ESPSLIA |
| | | (SEQ ID NO: 5); |
| Stress | saliva | Cortisol, chromogranin A, alpha-amylase, |
| | | secretary IgA, lysozyme |
| | | dehydro-androsteronesulfate; 17- |
| | | ketosteroidsulfate; dehydro- |
| | | epiandrostronesulfate; corticosteroid, 17- |
| | | hydroxycorticosteroid, growth hormone, |
| | | oxytocin |
| | miscellaneous | aldose reductase, apoptosis signal- |
| | | regulating kinase 1, aquaporin 5, beta- |
| | | endorphin, betaine GABA transporter, |
| | | caspase recruitment domain protein 9, |
| | | caspase 8, cyclin D, cyclooxygenase 2, |
| | | cytochrome P450, cytochrome c, c-fos, c- |
| | | jun, epidermal growth factor receptor, |
| | | ferritin, glucocorticoid receptor, glucose |
| | | regulated protein 58, glucose regulated |
| | | protein 75, glutathione S-transferase p, |
| | | GroEL, heat shock protein 25/27, heat |
| | | shock protein 40, heat shock protein 60, |
| | | heat shock protein 70, heat shock protein |
| | | 90, heat shock transcription factor-1, heme |
| | | oxygenase-1, interleukin 1β, interleukin 6, |
| | | interleukin 8, interleukin 10, interleukin |
| | | 12, laminin, leptin receptor, matrix |
| | | metalloproteinase 9, metallothionein, Mek- |
| | | 1, Mekk-1, inducible nitric oxide synthase, |
| | | peripheral benzodiazepine receptor, p38 |
| | | MAPK, salivary alpha amylase, SAPK, |
| | | serotonin, serotonin receptor, substance P, |
| | | superoxide dismutase Mn, superoxide |
| | | dismutase Cu/Zn, superoxide dismutase |
| | | EC, transforming growth factor β, tumor |
| | | suppressor p53, and vasoactive intestinal |
| | | peptide |
| Malnutrition | Saliva | sIgA |
| Nutritional status | miscellaneous | Prealbumin, Albumin, Retinol-binding |
| | | protein (RBP), Transferrin, Acylation- |
| | | Stimulating Protein (ASP), Adiponectin, |
| | | Agouti-Related Protein (AgRP), |
| | | Angiopoietin-like Protein 4 (ANGPTL4, |
| | | FIAF), C-peptide, AFABP (Adipocyte |
| | | Fatty Acid Binding Protein, FABP4), |
| | | Acylation-Stimulating Protein (ASP), |
| | | EFABP (Epidermal Fatty Acid Binding |
| | | Protein, FABP5), Glicentin, Glucagon, |
| | | Glucagon-Like Peptide-1, Glucagon-Like |
| | | Peptide-2, Ghrelin, Insulin, Leptin, Leptin |
| | | Receptor, PYY, RELMs, Resistin, and |
| | | sTfR (soluble Transferrin Receptor) |
| Energy balance | Serum | AMPK |
| (protein excretion)/ | Urine, sweat, | pre-albumin, retinol binding protein, urea |
| energy status/ | feces | |
| metabolic state | miscellaneous | cholesterol, lipoproteins, insulin, insulin C |
| | | peptide, IGF binding proteins, e.g. IGF- |
| | | BPl, liver enzymes |
| Diabetes | Miscellaneous | 11-8, CTSS, ITGB2, HLA-DRA, CD53, |
| | | PLAG27, or MMP9; RBP4; |
| | Urine | 8-iso-prostaglandin F2α (8-iso-PGF2α), |
| | Urine | 11-dehydro-thromboxane B₂ (TXM) |
| | | C-peptide |
| | Miscellaneous | Advanced glycosylation end products |
| | | (AGEs), 1,5-anhydroglucitol, NGPTL3 and 4 |
| | | autoantibodies (Zn transporter 8, glutamic |
| | | acid decarboxylase (GAD)) |
| | Urine (serum, | ATP-binding cassette, sub-family C |
| | etc.)- | (CFTR/MRP), member 8; ATP-binding |
| | miscellaneous | cassette, sub-family C (CFTR/MRP), |
| | | member 9; angiotensin I converting |
| | | enzyme (peptidyl-dipeptidase A) 1; |
| | | adenylate cyclase activating polypeptide 1 |
| | | (pituitary); adiponectin, C1Q and collagen |
| | | domain containing; adiponectin receptor 1; |
| | | adiponectin receptor 2; adrenomedullin; |
| | | adrenergic, beta-2-, receptor, surface; |
| | | advanced glycosylation end product- |
| | | specific receptor; agouti related protein |
| | | homolog (mouse); angiotensinogen (serpin |
| | | peptidase inhibitor, clade A, member 8); |
| | | angiotensin II receptor, type 1; angiotensin |
| | | II receptor-associated protein; alpha-2-HS- |
| | | glycoprotein; v-akt murine thymoma viral |
| | | oncogene homolog 1; v-akt murine |
| | | thymoma viral oncogene homolog 2; |
| | | albumin; Alstrom syndrome 1; archidonate |
| | | 12-lipoxygenase; ankyrin repeat domain |
| | | 23; apelin, AGTRL 1 Ligand; |
| | | apolipoprotein A-I; apolipoprotein A-II; |
| | | apolipoprotein B (including Ag(x) |
| | | antigen); apolipoprotein E; aryl |
| | | hydrocarbon receptor nuclear translocator; |
| | | Aryl hydrocarbon receptor nuclear |
| | | translocator-like; arrestin, beta 1; arginine |
| | | vasopressin (neurophysin II, antidiuretic |
| | | hormone, Diabetes insipidus, |
| | | neurohypophyseal); |
| | | bombesin receptor subtype 3; betacellulin; |
| | | benzodiazepine receptor (peripheral); |
| | | complement component 3; complement |
| | | component 4A (Rodgers blood group); |
| | | complement component 4B (Childo blood |
| | | group); complement component 5; |
| | | Calpain-10; cholecystokinin; |
| | | cholecystokinin (CCK)-A receptor; |
| | | chemokine (C-C motif) ligand 2; CD14 |
| | | molecule; CD163 molecule; CD36 |
| | | molecule (thrombospondin receptor); |
| | | CD38 molecule; CD3d molecule, delta |
| | | (CD3-TCR complex); CD3g molecule, |
| | | gamma (CD3-TCR complex); CD40 |
| | | molecule, TNF receptor superfamily |
| | | member 5; CD40 ligand (TNF superfamily, |
| | | member 5, hyper-IgM syndrome); CD68 |
| | | molecule; cyclin-dependent kinase 5; |
| | | complement factor D (adipsin); CASP8 |
| | | and FADD-like apoptosis regulator; Clock |
| | | homolog (mouse); chymase 1, mast cell; |
| | | cannabinoid receptor 1 (brain); |
| | | cannabinoid receptor 2 (macrophage); |
| | | cortistatin; carnitine palmitoyltransferase |
| | | I; carnitine palmitoyltransferase II; |
| | | complement component (3b/4b) receptor 1; |
| | | complement component (3d/Epstein Barr |
| | | virus) receptor 2; CREB binding protein |
| | | (Rubinstein-Taybi syndrome); C-reactive |
| | | protein, pentraxin-related; CREB regulated |
| | | transcription coactivator 2; colony |
| | | stimulating factor 1 (macrophage); |
| | | cathepsin B; cathepsin L; cytochrome |
| | | P450, family 19, subfamily A, polypeptide |
| | | 1; Dio-2, death inducer-obliterator 1; |
| | | dipeptidyl-peptidase 4 (CD26, adenosine |
| | | deaminase complexing protein 2); |
| | | epidermal growth factor (beta- |
| | | urogastrone); early growth response 1; |
| | | epididymal sperm binding protein 1; |
| | | ectonucleotide; |
| | | pyrophosphatase/phosphodiesterase 1; |
| | | E1A binding protein p300; coagulation |
| | | factor XIII, A1 polypeptide; coagulation |
| | | factor VIII, procoagulant component |
| | | (hemophilia A); fatty acid binding protein |
| | | 4, adipocyte; Fas (TNF receptor |
| | | superfamily, member 6); Fas ligand (TNF |
| | | superfamily, member 6); free fatty acid |
| | | receptor 1; fibrinogen alpha chain; |
| | | forkhead box A2; forkhead box O1A; |
| | | ferritin; glutamate decarboxylase 2; |
| | | galanin; gastrin; glucagon; glucokinase; |
| | | gamma-glutamyltransferase 1; growth |
| | | hormone 1; ghrelin/obestatin |
| | | preprohormone; gastric inhibitory |
| | | polypeptide; gastric inhibitory polypeptide |
| | | receptor; glucagon-like peptide 1 receptor; |
| | | guanine nucleotide binding protein (G |
| | | protein), beta polypeptide 3; glutamic- |
| | | pyruvate transaminase (alanine |
| | | aminotransferase); gastrin releasing peptide |
| | | (bombesin); gelsolin (amyloidosis, |
| | | Finnish type); hemoglobin; hemoglobin, |
| | | beta; hypocretin (orexin); neuropeptide; |
| | | precursor; hepatocyte growth factor |
| | | (hepapoietin A; scatter factor); hepatocyte |
| | | nuclear factor 4, alpha; haptoglobin; |
| | | hydroxysteroid (11-beta); dehydrogenase |
| | | 1; heat shock 70 kDa protein 1B; islet |
| | | amyloid polypeptide; intercellular |
| | | adhesion molecule 1 (CD54), human |
| | | rhinovirus receptor; interferon, gamma; |
| | | insulin-like growth factor 1 (somatomedin |
| | | C); insulin-like growth factor 2 |
| | | (somatomedin A); insulin-like growth |
| | | factor binding protein 1; insulin-like |
| | | growth factor binding protein 3; inhibitor |
| | | of kappa light polypeptide gene enhancer |
| | | in B-cells, kinase beta; interleukin 10; |
| | | interleukin 18 (interferon-gamma-inducing |
| | | factor); interleukin 1, alpha; interleukin 1, |
| | | beta; interleukin 1 receptor antagonist; |
| | | interleukin 2; interleukin 6 (interferon, beta |
| | | 2); interleukin 6 receptor; interleukin 8; |
| | | inhibin, beta A (activin A, activin AB |
| | | alpha polypeptide); insulin; insulin |
| | | receptor; insulin promoter factor-1; insulin |
| | | receptor substrate 1; insulin receptor |
| | | substrate-2; potassium inwardly-rectifying |
| | | channel, subfamily J, member 11; |
| | | potassium inwardly-rectifying channel, |
| | | subfamily J, member 8; klotho; kallikrein |
| | | B, plasma (Fletcher factor) 1; leptin |
| | | (obesity homolog, mouse); leptin receptor; |
| | | legumain; lipoprotein, Lp(a); lipoprotein |
| | | lipase; v-maf musculoaponeurotic |
| | | brosarcoma oncogene homolog A (avian); |
| | | mitogen-activated protein kinase 8; |
| | | interacting protein 1; mannose-binding |
| | | lectin (protein C) 2, soluble (opsonic |
| | | defect); melanocortin 4 receptor; melanin- |
| | | concentrating hormone receptor 1; matrix |
| | | metallopeptidase 12 (macrophage elastase); |
| | | matrix metallopeptidase 14 (membrane- |
| | | inserted); matrix metallopeptidase 2 |
| | | (gelatinase A, 72 kDa gelatinase, 72 kDa |
| | | type IV collagenase); matrix |
| | | metallopeptidase 9 (gelatinase B, 92 kDa |
| | | gelatinase, 92 kDa type IV collagenase); |
| | | nuclear receptor co-repressor 1; neurogenic |
| | | differentiation 1; nuclear factor of kappa |
| | | light polypeptide gene enhancer in B-cells |
| | | 1(p105); nerve growth factor, beta |
| | | polypeptide; non-insulin-dependent |
| | | Diabetes Mellitus (common, type 2) 1; |
| | | non-insulin-dependent Diabetes Mellitus |
| | | (common, type 2) 2; Noninsulin-dependent |
| | | Diabetes Mellitus 3; nischarin (imidazoline |
| | | receptor); NF-kappaB repressing factor; |
| | | neuronatin; nitric oxide synthase 2A; |
| | | Niemann-Pick disease, type C2; natriuretic |
| | | peptide precursor B; nuclear receptor |
| | | subfamily 1, group D, member 1; nuclear |
| | | respiratory factor 1; oxytocin, prepro- |
| | | (neurophysin I); purinergic receptor P2Y, |
| | | G-protein coupled, 10; purinergic receptor |
| | | P2Y, G-protein coupled, 12; purinergic |
| | | receptor P2Y, G-protein coupled, 2; |
| | | progestagen-associated endometrial; |
| | | protein (placental protein 14, pregnancy- |
| | | associated endometrial alpha-2-globulin, |
| | | alpha uterine protein); paired box gene 4; |
| | | pre-B-cell colony enhancing factor 1; |
| | | phosphoenolpyruvate carboxykinase 1 |
| | | (PEPCK1); proprotein convertase; |
| | | subtilisin/kexin type 1; placental growth |
| | | factor, vascular; endothelial growth factor- |
| | | related protein; phosphoinositide-3-kinase, |
| | | catalytic, alpha polypeptide; |
| | | phosphoinositide-3-kinase, regulatory |
| | | subunit 1 (p85 alpha); |
| | | phospholipase A2, group XIIA; |
| | | phospholipase A2, group IID; plasminogen |
| | | activator, tissue; patatin-like phospholipase |
| | | domain containing 2; proopiomelanocortin |
| | | (adrenocorticotropin/beta-lipotropin/alpha- |
| | | melanocyte stimulating hormone/beta- |
| | | melanocyte stimulating hormone/beta- |
| | | endorphin); paraoxonase 1 ESA, PON, |
| | | Paraoxonase; peroxisome proliferative |
| | | activated receptor, alpha; peroxisome |
| | | proliferative activated receptor, delta; |
| | | peroxisome proliferative activated |
| | | receptor, gamma; peroxisome proliferative |
| | | activated receptor, gamma, coactivator 1; |
| | | protein phosphatase 1, regulatory |
| | | (inhibitor) subunit 3A (glycogen and |
| | | sarcoplasmic reticulum binding subunit, |
| | | skeletal muscle); protein phosphatase 2A, |
| | | regulatory subunit B′ (PR 53); protein |
| | | kinase, AMP-activated, beta 1 non- |
| | | catalytic subunit; protein kinase, cAMP- |
| | | dependent, catalytic, alpha; protein kinase |
| | | C, epsilon; proteasome (prosome, |
| | | macropain) 26S subunit, non-ATPase, 9 |
| | | (Bridge-1); prostaglandin E synthase; |
| | | prostaglandin-endoperoxide synthase 2 |
| | | (prostaglandin G/H synthase and |
| | | cyclooxygenase); protein tyrosine |
| | | phosphatase, mitochondrial 1; Peptide YY |
| | | retinol binding protein 4, plasma (RBP4); |
| | | regenerating islet-derived 1 alpha |
| | | (pancreatic stone protein, pancreatic thread |
| | | protein); resistin; ribosomal protein S6 |
| | | kinase, 90 kDa, polypeptide 1; Ras-related |
| | | associated with Diabetes; serum amyloid |
| | | A1; selectin E (endothelial adhesion |
| | | molecule 1); serpin peptidase inhibitor, |
| | | clade A (alpha-1 antiproteinase, |
| | | antitrypsin), member 6; serpin peptidase |
| | | inhibitor, clade E (nexin, plasminogen |
| | | activator inhibitor type 1), member 1; |
| | | serum/glucocorticoid regulated kinase; sex |
| | | hormone-binding globulin; thioredoxin |
| | | interacting protein; |
| | | solute carrier family 2, member 10; solute |
| | | carrier family 2, member 2; solute carrier |
| | | family 2, member 4; solute carrier family 7 |
| | | (cationic amino acid transporter, y+ |
| | | system), member 1(ERR); SNF1-like |
| | | kinase 2; suppressor of cytokine signaling |
| | | 3; v-src sarcoma (Schmidt-Ruppin A-2) |
| | | viral oncogene homolog (avian); sterol |
| | | regulatory element binding transcription |
| | | factor 1; solute carrier family 2, member 4; |
| | | somatostatin receptor 2; somatostatin |
| | | receptor 5; transcription factor 1, hepatic; |
| | | LF-B1, hepatic nuclear factor (HNF1); |
| | | transcription factor 2, hepatic, LF-B3, |
| | | variant hepatic nuclear factor; transcription |
| | | factor 7-like 2 (T-cell specific, HMG-box); |
| | | transforming growth factor, beta 1 |
| | | (Camurati-Engelmann disease); |
| | | transglutaminase 2 (C polypeptide, protein- |
| | | glutamine-gamma-glutamyltransferase); |
| | | thrombospondin 1; thrombospondin, type I, |
| | | domain containing 1; tumor necrosis factor |
| | | (TNF superfamily, member 2); tumor |
| | | necrosis factor (TNF superfamily, member |
| | | 2); tumor necrosis factor receptor |
| | | superfamily, member 1A; tumor necrosis |
| | | factor receptor superfamily, member 1B; |
| | | tryptophan hydroxylase 2; thyrotropin- |
| | | releasing hormone; transient receptor |
| | | potential cation channel, subfamily V, |
| | | member 1; thioredoxin interacting protein; |
| | | thioredoxin reductase 2; urocortin 3 |
| | | (stresscopin); uncoupling protein 2 |
| | | (mitochondrial, proton carrier); upstream |
| | | transcription factor 1; urotensin 2; vascular |
| | | cell adhesion molecule 1; vascular |
| | | endothelial growth factor; vimentin; |
| | | vasoactive intestinal peptide; vasoactive |
| | | intestinal peptide receptor 1; vasoactive |
| | | intestinal peptide receptor 2; von |
| | | Willebrand factor; Wolfram syndrome 1 |
| | | (wolframin); X-ray repair complementing |
| | | defective repair in Chinese hamster cells 6; |
| | | c-peptide; cortisol; vitamin D3; estrogen; |
| | | estradiol; digitalis-like factor; |
| | | oxyntomodulin; dehydroepiandrosterone |
| | | sulfate (DHEAS); serotonin (5- |
| | | hydroxytryptamine); anti-CD38 |
| | | autoantibodies; gad65 autoantibody; |
| | | Angiogenin, ribonuclease, RNase A |
| | | family, 5; Hemoglobin A1c; Intercellular |
| | | adhesion molecule 3 (CD50); interleukin 6 |
| | | signal transducer (gp130, oncostatin M |
| | | receptor); selectin P (granule embrane |
| | | protein 140 kDa, antigen CD62); TIMP |
| | | metallopeptidase inhibitor; Proinsulin; |
| | | endoglin; |
| | | interleukin 2 receptor, beta; insulin-like |
| | | growth factor binding protein 2; insulin- |
| | | like growth factor 1 receptor; fructosamine, |
| | | N-acetyl-beta-d-glucosaminidase, |
| | | pentosidine, advanced glycation end |
| | | product, beta2-microglobulin, pyrraline |
| Metabolic | Serum | GFAP autoantibodies |
| syndrome/prediabetes | | |
| Alcohol | saliva | aminotransferases, gamma- |
| abuse/dependence | | glutamyltransferase, ethanol, ethyl |
| | | glucuronide, sialic acid, β-hexosaminidase |
| | | A, oral peroxidase, methanol, |
| | | diethylene/ethylene glycol, α-amylase, |
| | | clusterin, haptoglobin, heavy/light chains |
| | | of immunoglobulins and transferrin; α- |
| | | fucosidase (FUC), α-mannosidase (MAN), |
| | | β-galactosidase (GAL), and β- |
| | | glucuronidase (GLU) |
| Non-alcoholic fatty | miscellaneous | cytokeratin CK-18 (M65 antigen), caspase- |
| liver disease | | cleaved CK-18 (M30-antigen), resistin, |
| | | adiponectin, visfatin, insulin, tumor |
| | | necrosis factor-alpha (TNF-α), interleukin |
| | | 6 (IL-6), or interleukin 8 (IL-8) |
| | Serum | aspartate aminotransferase (AST) and |
| | | alanine aminotransferase (ALT); gamma- |
| | | glutamyltransferase (GGT), |
| | | immunoglobulin A, carbohydrate-deficient |
| | | transferrin (CDT), glutamic oxaloacetic |
| | | transaminase (GOT), glutamic pyruvic |
| | | transaminase (GPT), bilirubin |
| Cystic fibrosis | saliva | amylase |
| | | cathepsin-D, lactate dehydrogenase |
| Ectodermal dysplasia | saliva | alpha-amylase |
| sarcoidosis | serum | IL-6, TNF-α, IFN-α, IL-17, IP-10, MIG, |
| | | HGF, VEGF, TNF-RII, G-CSF, IFN-γ, |
| | | MCP-1, RANTES and IL-5 |
| Asthma | Saliva | eotaxin-1/CCL11, RANTES/CCL5, and |
| | | IL-5; IL-β, IL-6, MCP-1/CCL2, and IL- |
| | | 8/CXCL8; IP-10/CXCL10 |
| Periodontitis/dental | | aspartate aminotransferase (AST) and |
| caries | | alkaline phosphatase (ALP), uric acid and |
| | | albumin; 12-HETE; MMP-8, TIMP-1, and |
| | | ICTP |
| Muscle damage | Serum, urine | Myoglobin, creatine kinase (CK), lactate |
| | | dehydrogenase (LDH), aldolase, troponin, |
| | | carbonic anhydrase type 3 and fatty acid- |
| | | binding protein (FABP), transaminases |
| Infection | miscellaneous | IL-32, NXNL1, PSMA7, C6orf61, EMP1, |
| (Mycobacterium | | CLIC1, LACTB and DUSP3, LOC389541, |
| tuberculosis) | | MIDI IP 1, KLRC3, KLF9, FBXQ32, |
| | | C50RF29, CHUK, LOC652062, |
| | | C6ORF60, MTMR 11, sCD170; IFN- |
| | | gamma; IL-Iβ, IL-6, IL-8, IL-10, IL-12p70, |
| | | sCD4, SCD25, SCD26, sCD32b/c, SCD50, |
| | | SCD56, sCD66a, SCD83, sCD85j, SCD95, |
| | | SCD106, sCD120b, sCD121b, SCD127, |
| | | SCD154, SCD222, SCD226, sCDw329 |
| | | and TNF alpha; VEGF, AAT, CRP, IL- |
| | | IRA, TIMP-1, IL-18, A2Macro, |
| | | Haptoglobin ICAM-1, VCAM-1, SCF, IL- |
| | | 17, Fibrinogen, beta-2-macroglobulin, |
| | | TNF-alpha, C3 and TNFR2, GPR117, |
| | | TAZ, HSDL 1, HIP 1 (host); |
| Infection | saliva | MUC-5B and MUC 7 |
| (Helicobacter pylori) | | |
| Infection (Candida | saliva | Hsp70, calprotectin, histatins, mucins, |
| species) | | basic proline rich proteins and peroxidases |
| | | (host); |
| Infection (influenza) | miscellaneous | Hemagglutinin (H1), neuraminidase (N1); |
| | | C-reactive protein, [RNA:] DNA cross-link |
| | | repair 1A, PSO2 homolog, synaptonemal |
| | | complex protein 3, v-maf |
| | | musculoaponeurotic fibrosarcoma |
| | | oncogene family, chitinase 3-like 3, matrix |
| | | metalloproteinase 12, ATP-binding |
| | | cassette, sub-family E (OABP), member 1, |
| | | ATP-binding cassette, sub-family F |
| | | (GCN20), member 1, feminization 1 |
| | | homolog a (C. elegans), general |
| | | transcription factor II H. polypeptide 2, |
| | | forkhead box P1, zinc finger protein 282, |
| | | arginyl-tRNA synthetase-like, |
| | | Mitochondrial ribosomal protein L48, |
| | | ribosomal protein S4, X-linked, eukaryotic |
| | | translation elongation factor 1 alpha 1, |
| | | proteaseome (prosome, macropain) 28 |
| | | subunit 3, GLE1 RNA export mediator-like |
| | | (yeast), small nuclear ribonucleoprotein |
| | | polypeptide A′, cleavage and |
| | | polyadenylation specific factor 2, |
| | | ribosomal protein L27a,, thioredoxin |
| | | domain containing 4 (endoplasmic |
| | | reticulum), flap structure specific |
| | | endonuclease 1, ADP-ribosylation factor- |
| | | like 6 interacting protein 2, cytidine 5′- |
| | | triphosphate synthase 2, glutathione S- |
| | | transferase, mu 5, phospholipase D1, |
| | | aspartate-beta-hydroxylase, leukotriene A4 |
| | | hydrolase, cytochrome P450 family 17, |
| | | subfamily a, polypeptide 1, thioredoxin |
| | | interacting protein, carbonyl reductase 2, |
| | | alpha globin regulatory element containing |
| | | gene, male-specific lethal-2 homolog |
| | | (Drosophila), RAB1, member RAS |
| | | oncogene family, protein tyrosine |
| | | phosphatase, non-receptor type 21, |
| | | potassium voltage-gated channel, lsk- |
| | | related subfamily, gene 3, Bcl2-associated |
| | | athanogene 3, lymphocyte cytosolic protein |
| | | 2, pore forming protein-like, tumor |
| | | necrosis factor receptor superfamily, |
| | | member 19, filamin beta, microtubule-actin |
| | | crosslinking factor 1, keratin complex 1, |
| | | acidic, gene 18, keratin complex 1, acidic, |
| | | gene 19, mesoderm development candiate |
| | | 2, tubulin, alpha 4,, glutathione peroxidase |
| | | 1, integrin linked kinase, guanine |
| | | nucleotide binding protein, alpha inhibiting |
| | | 2, cyclin L2, tubulin, alpha 2, DEAD (Asp- |
| | | Glu-Ala-Asp) box polypeptide 5, |
| | | programmed cell death 4, proteasome |
| | | (prosome, macropain) 26S subunit, non- |
| | | ATPase 8, signal sequence receptor, beta, |
| | | RAD23b homolog (host); |
| Infection (HIV-1) | Urine, serum | p24, gp41, gp120 |
| Infection (Hepatitis B | miscellaneous | Core, Envelope, Surface (Ay), |
| virus) | | |
| Infection (Hepatitis C | miscellaneous | Core, NS3, NS4, NS5, |
| virus) | | |
| Infection (Hepatitis E | miscellaneous | orf2 3 KD, orf2 6 KD, orf3 3 KD |
| virus) | | |
| Infection (Vibrio | miscellaneous | Cholera Toxin |
| cholerae) | | |
| Infection | miscellaneous | Diphtheria toxin |
| (Corynebacterium | | |
| diphtheria) | | |
| Infection (Epstein- | miscellaneous | EA, VCA, NA |
| Barr virus) | | |
| Infection (Herpes | miscellaneous | gD |
| simplex virus HSV-1) | | |
| Infection (Herpes | miscellaneous | gG |
| simplex virus HSV-2) | | |
| Infection (Clostridium | miscellaneous | Tetanus toxin |
| tetani) | | |
| Infection (Treponema | miscellaneous | 15 kd, p47 |
| pallidum) | | |
| Infection (Entamoeba | saliva | M17 |
| histolytica) | | |
| Infection (Toxoplasma | serum | a2-HS glycoprotein and apB glycoprotein |
| gondii) | | (host); TGME49 052280, |
| | | TGME49_021500, TGME49J) 19630, |
| | | TGME49_061720 and TGME49_076220; |
| Infection (Dengue | miscellaneous | IL-10, fibrinogen, C4A, immunoglobulin, |
| virus) | | tropomyosin, albumin, SCSb-9 |
| | | complement complex (host); NS-1 |
| Infection | miscellaneous | stratifin, cullin 1, selenoprotein K, metal |
| (Streptococcus | | response element binding transcription |
| pneumonia) | | factor 2, prostaglandin E synthase 2, HLA- |
| | | B associated transcript 4, zinc finger |
| | | protein (C2H2 type) 276, GCIP-interacting |
| | | protein p29, mitochondrial ribosomal |
| | | protein L20, aryl hydrocarbon receptor |
| | | nuclear translocator-like, secretory carrier |
| | | membrane protein 1, nuclear receptor |
| | | subfamily 5, group A, member 2, NIMA |
| | | (never in mitosis gene a)-related expressed, |
| | | kinase 7, ribosomal protein L28, ribosomal |
| | | protein S25, lysosomal-associated protein |
| | | transmembrane 5, neural precursor cell |
| | | expressed, developmentally, down-regulted |
| | | gene 4, alpha glucosidase 2, alpha neutral |
| | | subunit, coatomer protein complex, subunit |
| | | beta 2 (beta prime), ribosomal protein L3, |
| | | NADH dehydrogenase (ubiquinone) 1 |
| | | alpha, subcomplex, assembly factor 1, |
| | | isoprenylcysteine carboxyl |
| | | methyltransferase,, cytoplasmic |
| | | polyadenylation element binding protein 3, |
| | | mannoside acetylglucosaminyltransferase |
| | | 1, RNA-binding region (RNP1, RRM) |
| | | containing 1,, folate receptor 4 (delta), |
| | | ATPase, H+ transporting, lysosomal 50/57 kDa, |
| | | V1, subunit H, zinc finger, DHHC |
| | | domain containing 6, phosphoribosyl |
| | | pyrophosphate synthetase-associated, |
| | | protein 2, |
| | | choline/ethanolaminephosphotransferase |
| | | 1,, solute carrier family 38, member 1, |
| | | ATP synthase, H+ transporting, |
| | | mitochondrial F0, complex, subunit f, |
| | | isoform 2, glucose phosphate isomerase 1, |
| | | 2′-5′ oligoadenylate synthetase 1A, |
| | | tyrosine hydroxylase, hemoglobin alpha, |
| | | adult chain 1, selenoprotein P, plasma, 1, |
| | | acetyl-Coenzyme A dehydrogenase, long- |
| | | chain, mannosidase, beta A, lysosomal,, |
| | | deltex 3 homolog (Drosophila), ras |
| | | homolog gene family, member AB, |
| | | estrogen receptor 1 (alpha), |
| | | phosphoglycerate kinase 1,, keratin |
| | | complex 2, basic, gene 8, emerin, |
| | | nucleoporin 153, formin 2, prothymosin |
| | | alpha, synapsin I,, cullin 4B, regulator of |
| | | chromosome condensation (RCC1) and, |
| | | BTB (POZ) domain containing protein 1,, |
| | | immediate early response 5, SAM domain |
| | | and HD domain, 1, tumor rejection antigen |
| | | gp96, lymphocyte antigen 6 complex, locus |
| | | E,, DAZ associated protein 2, general |
| | | transcription factor II I, RNA polymerase |
| | | II transcrptional coactivator, SWI/SNF- |
| | | related, matrix-associated actin-dependent, |
| | | regulator of chromatin, subfamily a, |
| | | containing DEAD/H, box 1, structure |
| | | specific recognition protein 1, ankyrin |
| | | repeat and FYVE domain containing 1, |
| | | SET translocation, myocyte enhancer |
| | | factor 2A, homeo box D9, H2A histone |
| | | family, member Z, cellular nucleic acid |
| | | binding protein,, golgi reassembly |
| | | stacking protein 2, cathepsin L, eukaryotic |
| | | translation initiation factor 5, ubiquitin |
| | | specific protease 9, X chromosome, |
| | | proteasome (prosome, macropain) subunit, |
| | | alpha type 7, pescadillo homolog 1, |
| | | containing BRCT domain, (zebrafish), |
| | | heterogeneous nuclear ribonucleoprotein |
| | | K, DEAD (Asp-Glu-Ala-Asp) box |
| | | polypeptide 52, sorting nexin 5, cathepsin |
| | | B, DnaJ (Hsp40) homolog, subfamily B, |
| | | member 9, ribosomal protein s3a,, |
| | | cytoplasmic polyadenylation element |
| | | binding protein 4,5′-3′ exoribonuclease |
| | | 2, small nuclear ribonucleoprotein |
| | | polypeptide F,, arachidonate 5- |
| | | lipoxygenase activating protein, |
| | | cytochrome c oxidase, subunit VIc, |
| | | RIKubiquinol cytochrome c reductase core |
| | | protein 2, lactate dehydrogenase 2, B |
| | | chain, ubiquinol-cytochrome c reductase |
| | | core protein 1, ATP synthase, H+ |
| | | transporting, mitochondrial F0, complex, |
| | | subunit b, isoform 1, microsomal |
| | | glutathione S-transferase 1, ras homolog |
| | | gene family, member A, RAB7, member |
| | | RAS oncogene family, EGF-like module |
| | | containing, mucin-like, hormone, receptor- |
| | | like sequence 1, annexin A6, mitogen |
| | | activated protein kinase 3, tyrosine kinase, |
| | | non-receptor, 2, villin 2, tubulin, beta 5, |
| | | catenin src (host); Pneumolysin, |
| | | pneumococcal histidine triad D (PhtD), |
| | | pneumococcal histidine triad E (PhtE), |
| | | LytB, and pneumococcal choline-binding |
| | | protein A (PcpA) |
| Infection | miscellaneous | DnaK, L7/L12, P1, exotoxin |
| (Mycoplasma | | |
| pneumonia) | | |
| Infection | miscellaneous | gyrA, 16S rDNA, or flaA/flaB |
| (Campylobacter | | |
| jejuni) | | |
| Infection (Bacillus | miscellaneous | Lethal factor, HtrA (BA3660), NlpC/P60- |
| anthracis) | | domain endopeptidase (BA1952), BA0796 |
| | | locus (BA0796), SAP |
| Infection (West Nile | miscellaneous | |
| virus) | | |
| Infection (Human | miscellaneous | E6, E7 |
| papilloma virus) | | |
| Infection | urine | RNase 7 (host); |

In some instances, the present method is used to inform the subject from whom the sample is derived about a health condition thereof. Health conditions that may be diagnosed or measured by the present method, device and system include, but are not limited to: chemical balance; nutritional health; exercise; fatigue; sleep; stress; prediabetes; allergies; aging; exposure to environmental toxins, pesticides, herbicides, synthetic hormone analogs; pregnancy; menopause; and andropause. The following Table 3 provides a list of biomarker that can be detected using the present signal-amplifying nanosensor (when used in conjunction with an appropriate monoclonal antibody, nucleic acid, or other capture agent), and their associated health conditions.

TABLE 3
Diagnostic Markers

| Health Condition | Source | Marker |
|---|---|---|
| Diabetes | Saliva | pIgR, Arp 3, CA VI, and IL-1Ra; PLS-2, |
| | | LEI, and IGJ chain, resistin |
| | miscellaneous | ATP-binding cassette, sub-family C |
| | | (CFTR/MRP), member 8; ATP-binding |
| | | cassette, sub-family C (CFTR/MRP), |
| | | member 9; angiotensin I converting enzyme |
| | | (peptidyl-dipeptidase A) 1; adenylate cyclase |
| | | activating polypeptide 1 (pituitary); |
| | | adiponectin, C1Q and collagen domain |
| | | containing; adiponectin receptor 1; |
| | | adiponectin receptor 2; adrenomedullin; |
| | | adrenergic, beta-2-, receptor, surface; |
| | | advanced glycosylation end product-specific |
| | | receptor; agouti related protein homolog |
| | | (mouse); angiotensinogen (serpin peptidase |
| | | inhibitor, clade A, member 8); angiotensin II |
| | | receptor, type 1; angiotensin II receptor- |
| | | associated protein; alpha-2-HS-glycoprotein; |
| | | v-akt murine thymoma viral oncogene |
| | | homolog 1; v-akt murine thymoma viral |
| | | oncogene homolog 2; albumin; Alstrom |
| | | syndrome 1; archidonate 12-lipoxygenase; |
| | | ankyrin repeat domain 23; apelin, AGTRL 1 |
| | | Ligand; apolipoprotein A-I; apolipoprotein |
| | | A-II; apolipoprotein B (including Ag(x) |
| | | antigen); apolipoprotein E; aryl hydrocarbon |
| | | receptor nuclear translocator; Aryl |
| | | hydrocarbon receptor nuclear translocator- |
| | | like; arrestin, beta 1; arginine vasopressin |
| | | (neurophysin II, antidiuretic hormone, |
| | | Diabetes insipidus, neurohypophyseal); |
| | | bombesin receptor subtype 3; betacellulin; |
| | | benzodiazepine receptor (peripheral); |
| | | complement component 3; complement |
| | | component 4A (Rodgers blood group); |
| | | complement component 4B (Childo blood |
| | | group); complement component 5; Calpain- |
| | | 10; cholecystokinin; cholecystokinin (CCK)- |
| | | A receptor; chemokine (C-C motif) ligand 2; |
| | | CD14 molecule; CD163 molecule; CD36 |
| | | molecule (thrombospondin receptor); CD38 |
| | | molecule; CD3d molecule, delta (CD3-TCR |
| | | complex); CD3g molecule, gamma (CD3- |
| | | TCR complex); CD40 molecule, TNF |
| | | receptor superfamily member 5; CD40 |
| | | ligand (TNF superfamily, member 5, hyper- |
| | | IgM syndrome); CD68 molecule; cyclin- |
| | | dependent kinase 5; complement factor D |
| | | (adipsin); CASP8 and FADD-like apoptosis |
| | | regulator; Clock homolog (mouse); chymase |
| | | 1, mast cell; cannabinoid receptor 1 (brain); |
| | | cannabinoid receptor 2 (macrophage); |
| | | cortistatin; carnitine palmitoyltransferase I; |
| | | carnitine palmitoyltransferase II; |
| | | complement component (3b/4b) receptor 1; |
| | | complement component (3d/Epstein Barr |
| | | virus) receptor 2; CREB binding protein |
| | | (Rubinstein-Taybi syndrome); C-reactive |
| | | protein, pentraxin-related; CREB regulated |
| | | transcription coactivator 2; colony |
| | | stimulating factor 1 (macrophage); cathepsin |
| | | B; cathepsin L; cytochrome P450, family |
| | | 19, subfamily A, polypeptide 1; Dio-2, death |
| | | inducer-obliterator 1; dipeptidyl-peptidase 4 |
| | | (CD26, adenosine deaminase complexing |
| | | protein 2); epidermal growth factor (beta- |
| | | urogastrone); early growth response 1; |
| | | epididymal sperm binding protein 1; |
| | | ectonucleotide; |
| | | pyrophosphatase/phosphodiesterase 1; E1A |
| | | binding protein p300; coagulation factor |
| | | XIII, A1 polypeptide; coagulation factor |
| | | VIII, procoagulant component (hemophilia |
| | | A); fatty acid binding protein 4, adipocyte; |
| | | Fas (TNF receptor superfamily, member 6); |
| | | Fas ligand (TNF superfamily, member 6); |
| | | free fatty acid receptor 1; fibrinogen alpha |
| | | chain; forkhead box A2; forkhead box O1A; |
| | | ferritin; glutamate decarboxylase 2; galanin; |
| | | gastrin; glucagon; glucokinase; gamma- |
| | | glutamyltransferase 1; growth hormone 1; |
| | | ghrelin/obestatin preprohormone; gastric |
| | | inhibitory polypeptide; gastric inhibitory |
| | | polypeptide receptor; glucagon-like peptide |
| | | 1 receptor; guanine nucleotide binding |
| | | protein (G protein), beta polypeptide 3; |
| | | glutamic-pyruvate transaminase (alanine |
| | | aminotransferase); gastrin releasing peptide |
| | | (bombesin); gelsolin (amyloidosis, Finnish |
| | | type); hemoglobin; hemoglobin, beta; |
| | | hypocretin (orexin); neuropeptide; precursor; |
| | | hepatocyte growth factor (hepapoietin A; |
| | | scatter factor); hepatocyte nuclear factor 4, |
| | | alpha; haptoglobin; hydroxysteroid (11- |
| | | beta); dehydrogenase 1; heat shock 70 kDa |
| | | protein 1B; islet amyloid polypeptide; |
| | | intercellular adhesion molecule 1 (CD54), |
| | | human rhinovirus receptor; interferon, |
| | | gamma; insulin-like growth factor 1 |
| | | (somatomedin C); insulin-like growth factor |
| | | 2 (somatomedin A); insulin-like growth |
| | | factor binding protein 1; insulin-like growth |
| | | factor binding protein 3; inhibitor of kappa |
| | | light polypeptide gene enhancer in B-cells, |
| | | kinase beta; interleukin 10; interleukin 18 |
| | | (interferon-gamma-inducing factor); |
| | | interleukin 1, alpha; interleukin 1, beta; |
| | | interleukin 1 receptor antagonist; interleukin |
| | | 2; interleukin 6 (interferon, beta 2); |
| | | interleukin 6 receptor; interleukin 8; inhibin, |
| | | beta A (activin A, activin AB alpha |
| | | polypeptide); insulin; insulin receptor; |
| | | insulin promoter factor-1; insulin receptor |
| | | substrate 1; insulin receptor substrate-2; |
| | | potassium inwardly-rectifying channel, |
| | | subfamily J, member 11; potassium |
| | | inwardly-rectifying channel, subfamily J, |
| | | member 8; klotho; kallikrein B, plasma |
| | | (Fletcher factor) 1; leptin (obesity homolog, |
| | | mouse); leptin receptor; legumain; |
| | | lipoprotein, Lp(a); lipoprotein lipase; v-maf |
| | | musculoaponeurotic brosarcoma oncogene |
| | | homolog A (avian); |
| | | mitogen-activated protein kinase 8; |
| | | interacting protein 1; mannose-binding lectin |
| | | (protein C) 2, soluble (opsonic defect); |
| | | melanocortin 4 receptor; melanin- |
| | | concentrating hormone receptor 1; matrix |
| | | metallopeptidase 12 (macrophage elastase); |
| | | matrix metallopeptidase 14 (membrane- |
| | | inserted); matrix metallopeptidase 2 |
| | | (gelatinase A, 72 kDa gelatinase, 72 kDa |
| | | type IV collagenase); matrix |
| | | metallopeptidase 9 (gelatinase B, 92 kDa |
| | | gelatinase, 92 kDa type IV collagenase); |
| | | nuclear receptor co-repressor 1; neurogenic |
| | | differentiation 1; nuclear factor of kappa |
| | | light polypeptide gene enhancer in B-cells |
| | | 1(p105); nerve growth factor, beta |
| | | polypeptide; non-insulin-dependent Diabetes |
| | | Mellitus (common, type 2) 1; non-insulin- |
| | | dependent Diabetes Mellitus (common, type |
| | | 2) 2; Noninsulin-dependent Diabetes |
| | | Mellitus 3; nischarin (imidazoline receptor); |
| | | NF-kappaB repressing factor; neuronatin; |
| | | nitric oxide synthase 2A; Niemann-Pick |
| | | disease, type C2; natriuretic peptide |
| | | precursor B; nuclear receptor subfamily 1, |
| | | group D, member 1; nuclear respiratory |
| | | factor 1; oxytocin, prepro-(neurophysin I); |
| | | purinergic receptor P2Y, G-protein coupled, |
| | | 10; purinergic receptor P2Y, G-protein |
| | | coupled, 12; purinergic receptor P2Y, G- |
| | | protein coupled, 2; progestagen-associated |
| | | endometrial; protein (placental protein 14, |
| | | pregnancy-associated endometrial alpha-2- |
| | | globulin, alpha uterine protein); paired box |
| | | gene 4; pre-B-cell colony enhancing factor 1; |
| | | phosphoenolpyruvate carboxykinase 1 |
| | | (PEPCK1); proprotein convertase; |
| | | subtilisin/kexin type 1; placental growth |
| | | factor, vascular; endothelial growth factor- |
| | | related protein; phosphoinositide-3-kinase, |
| | | catalytic, alpha polypeptide; |
| | | phosphoinositide-3-kinase, regulatory |
| | | subunit 1 (p85 alpha); |
| | | phospholipase A2, group XIIA; |
| | | phospholipase A2, group IID; plasminogen |
| | | activator, tissue; patatin-like phospholipase |
| | | domain containing 2; proopiomelanocortin |
| | | (adrenocorticotropin/beta-lipotropin/alpha- |
| | | melanocyte stimulating hormone/beta- |
| | | melanocyte stimulating hormone/beta- |
| | | endorphin); paraoxonase 1 ESA, PON, |
| | | Paraoxonase; peroxisome proliferative |
| | | activated receptor, alpha; peroxisome |
| | | proliferative activated receptor, delta; |
| | | peroxisome proliferative activated receptor, |
| | | gamma; peroxisome proliferative activated |
| | | receptor, gamma, coactivator 1; protein |
| | | phosphatase 1, regulatory |
| | | (inhibitor) subunit 3A (glycogen and |
| | | sarcoplasmic reticulum binding subunit, |
| | | skeletal muscle); protein phosphatase 2A, |
| | | regulatory subunit B′ (PR 53); protein |
| | | kinase, AMP-activated, beta 1 non-catalytic |
| | | subunit; protein kinase, cAMP-dependent, |
| | | catalytic, alpha; protein kinase C, epsilon; |
| | | proteasome (prosome, macropain) 26S |
| | | subunit, non-ATPase, 9 (Bridge-1); |
| | | prostaglandin E synthase; prostaglandin- |
| | | endoperoxide synthase 2 (prostaglandin G/H |
| | | synthase and cyclooxygenase); protein |
| | | tyrosine phosphatase, mitochondrial 1; |
| | | Peptide YY retinol binding protein 4, plasma |
| | | (RBP4); regenerating islet-derived 1 alpha |
| | | (pancreatic stone protein, pancreatic thread |
| | | protein); resistin; ribosomal protein S6 |
| | | kinase, 90 kDa, polypeptide 1; Ras-related |
| | | associated with Diabetes; serum amyloid A1; |
| | | selectin E (endothelial adhesion molecule 1); |
| | | serpin peptidase inhibitor, clade A (alpha-1 |
| | | antiproteinase, antitrypsin), member 6; |
| | | serpin peptidase inhibitor, clade E (nexin, |
| | | plasminogen activator inhibitor type 1), |
| | | member 1; serum/glucocorticoid regulated |
| | | kinase; sex hormone-binding globulin; |
| | | thioredoxin interacting protein; |
| | | solute carrier family 2, member 10; solute |
| | | carrier family 2, member 2; solute carrier |
| | | family 2, member 4; solute carrier family 7 |
| | | (cationic amino acid transporter, y+ system), |
| | | member 1(ERR); SNF1-like kinase 2; |
| | | suppressor of cytokine signaling 3; v-src |
| | | sarcoma (Schmidt-Ruppin A-2) viral |
| | | oncogene homolog (avian); sterol regulatory |
| | | element binding transcription factor 1; |
| | | solute carrier family 2, member 4; |
| | | somatostatin receptor 2; somatostatin |
| | | receptor 5; transcription factor 1, hepatic; |
| | | LF-B1, hepatic nuclear factor (HNF1); |
| | | transcription factor 2, hepatic, LF-B3, |
| | | variant hepatic nuclear factor; transcription |
| | | factor 7-like 2 (T-cell specific, HMG-box); |
| | | transforming growth factor, beta 1 |
| | | (Camurati-Engelmann disease); |
| | | transglutaminase 2 (C polypeptide, protein- |
| | | glutamine-gamma-glutamyltransferase); |
| | | thrombospondin 1; thrombospondin, type I, |
| | | domain containing 1; tumor necrosis factor |
| | | (TNF superfamily, member 2); tumor |
| | | necrosis factor (TNF superfamily, member |
| | | 2); tumor necrosis factor receptor |
| | | superfamily, member 1A; tumor necrosis |
| | | factor receptor superfamily, member 1B; |
| | | tryptophan hydroxylase 2; thyrotropin- |
| | | releasing hormone; transient receptor |
| | | potential cation channel, subfamily V, |
| | | member 1; thioredoxin interacting protein; |
| | | thioredoxin reductase 2; urocortin 3 |
| | | (stresscopin); uncoupling protein 2 |
| | | (mitochondrial, proton carrier); upstream |
| | | transcription factor 1; urotensin 2; vascular |
| | | cell adhesion molecule 1; vascular |
| | | endothelial growth factor; vimentin; |
| | | vasoactive intestinal peptide; vasoactive |
| | | intestinal peptide receptor 1; vasoactive |
| | | intestinal peptide receptor 2; von Willebrand |
| | | factor; Wolfram syndrome 1 (wolframin); X- |
| | | ray repair complementing defective repair in |
| | | Chinese hamster cells 6; c-peptide; cortisol; |
| | | vitamin D3; estrogen; estradiol; digitalis-like |
| | | factor; oxyntomodulin; |
| | | dehydroepiandrosterone sulfate (DHEAS); |
| | | serotonin (5-hydroxytryptamine); anti-CD38 |
| | | autoantibodies; gad65 autoantibody; |
| | | Angiogenin, ribonuclease, RNase A family, |
| | | 5; Hemoglobin A1c; Intercellular adhesion |
| | | molecule 3 (CD50); interleukin 6 signal |
| | | transducer (gp130, oncostatin M receptor); |
| | | selectin P (granule embrane protein 140 kDa, |
| | | antigen CD62); TIMP metallopeptidase |
| | | inhibitor; Proinsulin; endoglin; |
| | | interleukin 2 receptor, beta; insulin-like |
| | | growth factor binding protein 2; insulin-like |
| | | growth factor 1 receptor; fructosamine, N- |
| | | acetyl-beta-d-glucosaminidase, pentosidine, |
| | | advanced glycation end product, beta2- |
| | | microglobulin, pyrraline |
| Metabolic | Serum | GFAP autoantibodies |
| syndrome/prediabetes | | |
| Kidney | saliva | Lactoferrin, uric acid, cortisol, alpha- |
| failure/disease | | amylase |
| | miscellaneous | ADBP-26, NHE3, KIM-1, |
| | | glutamyltransferase, N-acetyl-beta-D- |
| | | glucosaminidase, lysozyme, NGAL, L- |
| | | FABP, bikunin, urea, prostaglandins, |
| | | creatinine, alpha-1-microglobulin, retinol |
| | | binding protein, glutathione-S-transferases, |
| | | adiponectin, beta-2-macroglobuin, calbindin- |
| | | D, cysteine-rich angiogenic inducer 61, |
| | | endothelial/epithial growth factors, alpha-1- |
| | | acid glycoprotein (orosomucoid), |
| | | prealbumin, modified albumin, albumin, |
| | | transferrin, alpha-1-lipoprotein, alpha-1- |
| | | antitrypsin matrix metalloproteinases |
| | | (MMPs), alpha-1-fetoprotein, Tamm |
| | | Horsfall protein, homoarginine, interleukin |
| | | 18, monocyte chemotactic protein-1 (MCP- |
| | | 1), Lipocalin, VCAN, NRP1, CCL2, CCL19, |
| | | COL3A1, GZMM, alpha-galactosidase, |
| | | casein kinase 2, IP-10, Mig, 1-TAC, MIP-1α, |
| | | MIP-3α, and MIP-1β, alpha-2-glycoprotein- |
| | | Zinc, leucine-rich alpha-2-glycoprotein, |
| | | uromodulin, Pacsin 2, hepcidin-20, hepcidin- |
| | | 25, AIF-2, urinary type-IV collagen, |
| | | lipocalin-type prostaglandin D synthase (L- |
| | | PGDS), urinary neutrophil gelatinase- |
| | | associated lipocalin (uNGAL), Annexin A1, |
| | | Rab23, Shh, Ihh, Dhh, PTCH1, PTCH2, |
| | | SMO, Gli1, Gli2, Gli3, TLR4, cystatin C, |
| | | AQP1, AQP2, AQP3, NKCC2, NaPill, |
| | | DAHKSEVAHRFKD |
| | | [RNA:] SLC12A1, UMOD, vWF, MMP1, |
| | | MMP3, SLC22A6, SLC22A 8, SLC22A 12, |
| | | podocin, cubulin, LRP2, AQP9, and |
| | | albumin, carcinoembryonic antigen (CEA), |
| | | mucin, alpha-fetoprotein, tyrosinase, |
| | | melanoma associated antigen, mutated tumor |
| | | protein 53, p21, PUMA, prostate-specific |
| | | antigen (PSA) or thyroglobulin, von |
| | | Willebrand factor (VWF), thrombin, factor |
| | | VIII, plasmin, fibrin, osteopontin (SPP1), |
| | | Rab23, Shh, Ihh, Dhh, PTCH1, PTCH2, |
| | | SMO, Gli1, Gli2, Gli3 |
| Liver failure/disease | miscellaneous | Carnitine; Cholic Acid; Chenodeoxycholic, |
| | | Deoxycholic, Lithocholic, Glycocholic; |
| | | Prostaglandin E2; 13,14-dihydro-15-keto |
| | | Prostaglandin A2; Prostaglandin B2; |
| | | Prostaglandin F2a; 15-keto-Prostaglandin |
| | | F2α; 6-keto-Prostaglandin F1α; |
| | | Thromboxane B2; 11-dehydro-Thromboxane |
| | | B2; Prostaglandin D2; Prostaglandin J2; |
| | | 15-deoxy-Δ12,14-Prostaglandin J2; 11β- |
| | | Prostaglandin F2α; 5(S)- |
| | | Hydroxyeicosatetraenoic acid; 5(S)- |
| | | Hydroxyeicosapentaenoic acid; Leukotriene |
| | | B4; Leukotriene B5; Leukotriene C4; |
| | | Leukotriene D4; Leukotriene E4; |
| | | Leukotriene F4; 12(S)- |
| | | Hydroxyeicosatetraenoic acid; 12(S)- |
| | | Hydroxyeicosapentaenoic acid; 15(S)- |
| | | Hydroxyeicosatetraenoic acid; 15(S)- |
| | | Hydroxyeicosapentaenoic acid; Lipoxin A4; |
| | | 8(S)-Hydroxyeicosatetraenoic acid; 9- |
| | | Hydroxyeicosatetraenoic acid; 11- |
| | | Hydroxyeicosatetraenoic acid; 8-iso- |
| | | Prostaglandin F2α; 9- |
| | | Hydroxyoctadecadienoic acid; 13- |
| | | Hydroxyoctadecadienoic acid; 20(S)- |
| | | Hydroxyeicosatetraenoic acid; 9,10- |
| | | Epoxyoctadecenoic acid; 12,13- |
| | | Epoxyoctadecenoic acid; 12,13- |
| | | Dihydroxyoctadecenoic acid; 5,6- |
| | | Epoxyeicosatrienoic acid; 11,12- |
| | | Epoxyeicosatrienoic acid; 14,15- |
| | | Epoxyeicosatrienoic acid; 5,6- |
| | | Dihydroxyeicosatrienoic acid; 8,9- |
| | | Dihydroxyeicosatrienoic acid; 11,12- |
| | | Dihydroxyeicosatrienoic acid; 14,15- |
| | | Dihydroxyeicosatrienoic acid; 14,15- |
| | | Epoxyeicosatetraenoic acid; 17,18- |
| | | Epoxyeicosatetraenoic acid; 14,15- |
| | | Dihydroxyeicosatetraenoic acid; 17,18- |
| | | Dihydroxyeicosatetraenoic acid; 19,20- |
| | | Dihydroxydocosapentaenoic acid; |
| | | diacetylspermine, hemopexin, TLR4 |
| Heart failure | miscellaneous | SFRP-3, NT-proBNP, troponin T, |
| | | SKITHRIHWESASLL (SEQ ID NO: 6), |
| | | AHKSEVAHRFK (SEQ ID NO: 7), |
| | | uroguanylin, BNP |
| Cardiovascular health | miscellaneous | miR-378, miR-497, miR-21, miR-15b, miR- |
| | | 99a, miR 29a, miR-24, miR-30b, miR-29c, |
| | | miR-331.3p, miR-19a, miR-22, miR-126, |
| | | let-7b, miR-502.3, and miR-652 |
| | | IL-16, sFas, Fas ligand, MCP-3, HGF, |
| | | CTACK, EOTAXIN, adiponectin, IL-18, |
| | | TIMP.4, TIMP.1, CRP, VEGF, and EGF |
| | saliva | C-reactive protein (CRP); myoglobin |
| | | (MYO), creatinine kinase myocardial band |
| | | (CK-MB), cardiac troponins (cTn), and |
| | | myeloperoxidase; TNF-α, and MMP-9; |
| | | CD40 |
| High blood pressure | saliva | lysozyme |
| Tiredness/fatigue | urine | endorepellin |
| | saliva | PPGKPQGPPPQGGNQPQGPPPPPGKPQ |
| | | (SEQ ID NO: 1); |
| | | GNPQGPSPQGGNKPQGPPPPPGKPQ |
| | | (SEQ ID NO: 2); |
| | | SPPGKPQGPPQQEGNKPQGPPPPGKPQ |
| | | (SEQ ID NO: 3) |
| | urine | human herpesvirus 6, human herpesvirus 7, |
| | | human cytomegalovirus, and Epstein-Barr |
| | | virus (EBV) |
| | miscellaneous | GGHPPPP (SEQ ID NO: 4), ESPSLIA (SEQ |
| | | ID NO: 5); |
| Malnutrition | Saliva | sIgA |
| Depressive disorder | miscellaneous | Secretogranin, VGF |
| Alzheimer's disease | CSF, serum, | β-amyloid(1-42), β-amyloid(1-40), tau, |
| | saliva | phosphor-tau-181 |
| Stress | saliva | Cortisol, dehydro-androsteronesulfate; 17- |
| | | ketosteroidsulfate; dehydro- |
| | | epiandrostronesulfate; corticosteroid, 17- |
| | | hydroxycorticosteroid, chromogranin A, |
| | | alpha-amylase, secretary IgA, lysozyme, |
| | | growth hormone, oxytocin |
| | miscellaneous | aldose reductase, apoptosis signal-regulating |
| | | kinase 1, aquaporin 5, beta-endorphin, |
| | | betaine GABA transporter, caspase |
| | | recruitment domain protein 9, caspase 8, |
| | | cyclin D, cyclooxygenase 2, cytochrome |
| | | P450, cytochrome c, c-fos, c-jun, epidermal |
| | | growth factor receptor, ferritin, |
| | | glucocorticoid receptor, glucose regulated |
| | | protein 58, glucose regulated protein 75, |
| | | glutathione S-transferase p, GroEL, heat |
| | | shock protein 25/27, heat shock protein 40, |
| | | heat shock protein 60, heat shock protein 70, |
| | | heat shock protein 90, heat shock |
| | | transcription factor-1, heme oxygenase-1, |
| | | interleukin 1β, interleukin 6, interleukin 8, |
| | | interleukin 10, interleukin 12, laminin, leptin |
| | | receptor, matrix metalloproteinase 9, |
| | | metallothionein, Mek-1, Mekk-1, inducible |
| | | nitric oxide synthase, peripheral |
| | | benzodiazepine receptor, p38 MAPK, |
| | | salivary alpha amylase, SAPK, serotonin, |
| | | serotonin receptor, substance P, superoxide |
| | | dismutase Mn, superoxide dismutase Cu/Zn, |
| | | superoxide dismutase EC, transforming |
| | | growth factor β, tumor suppressor p53, and |
| | | vasoactive intestinal peptide |
| Circadian rhythm | saliva | melatonin |
| Bone turnover/ | Urine | Pyridinoline, deoxypyridinoline, collagen |
| Osteoporosis | | type 1 corss-linked N-telopeptide (NTX), |
| | | collagen type 1 corss-linked C-telopeptide |
| | | (CTX), bone sialoprotein (BSP), Tartrate- |
| | | resistant acid phosphatase 5b |
| | saliva | deoxypyridinium (D-PYR) and osteocalcin |
| | | (OC), hepatocyte growth factor and |
| | | interleukin-1 beta |
| Muscle damage | Serum, urine | Myoglobin, creatine kinase (CK), lactate |
| | | dehydrogenase (LDH), aldolase, troponin, |
| | | carbonic anhydrase type 3 and fatty acid- |
| | | binding protein (FABP), transaminases |
| Exercise/athletic | sweat | urea |
| activity | serum | Myostatin, follistatin-like related gene |
| | saliva | testosterone |
| Performance | miscellaneous | interleukin-6, interleukin-1 beta, G-CSF, |
| enhancement | | interferon-gamma, interleukin-8, interleukin- |
| | | 9, MCP-1, MIP-beta, and/or TNF alpha |
| Energy balance | Serum | AMPK |
| (protein excretion)/ | Urine, sweat, | pre-albumin, retinol binding protein, urea |
| energy status/ | feces | |
| metabolic state | miscellaneous | cholesterol, lipoproteins, insulin, insulin C |
| | | peptide, IGF binding proteins, e.g. IGF-BP1, |
| | | liver enzymes |
| Growth | Saliva | IGF-1 |
| Andropause | saliva | testosterone; testosterone precursors such as |
| | | pregnenolone, progesterone, 17- |
| | | hydroxypregnenolone, 17- |
| | | hydroxyprogesterone, |
| | | dehydroepiandrosterone (DHEA) and delta- |
| | | 4-androstene-3,17-dione; testosterone and |
| | | dihydrotestosterone metabolites such as the |
| | | 17-ketosteroids androsterone and |
| | | etiocholanolone, polar metabolites in the |
| | | form of diols, triols, and conjugates, as well |
| | | estradiol, estrogens, androsteindione, |
| | | cortisol, DHEA, FSH (follicle stimulating |
| | | hormone), LH (luteinizing hormone), and |
| | | GnRH (gonadotropin-releasing hormone) |
| Menopause | Saliva | Follicle stimulating hormone (FSH) |
| | | Estrogen and progesterone, testosterone, free |
| | | testosterone, and dehydroepiandrosterone |
| | | sulfate |
| | | (DHEAS), cortisol and dehydroepi |
| | | androsterone (DHEA) |
| Pregnancy/fetal | Saliva | progesterone |
| development | urine | human chorionic gonadotropin, |
| | | Levonorgestrel, alpha-fetoprotein |
| | serum | estradiol |
| Breast cancer | urine | 47D10 antigen, PTCD2, SLC25A20, |
| | | NFKB2, RASGRP2, PDE7A, MLL, |
| | | PRKCE, GPATC3, PRIC285 and GSTA4, |
| | | MIPEP, PLCB2, SLC25A19, DEF6, |
| | | ZNF236, C18orf22, COX7A2, DDX11, |
| | | TOP3A, C9orf6, UFC1, PFDN2, KLRD1, |
| | | LOC643641, HSP90AB1, CLCN7, |
| | | TNFAIP2, PRKCE, MRPL40, FBF1, |
| | | ANKRD44, CCT5, USP40, UBXD4, |
| | | LRCH1, MRPL4, SCCPDH, STX6, |
| | | LOC284184, FLJ23235, GPATC3, CPSF4, |
| | | CREM, HIST1H1D, HPS4, FN3KRP, |
| | | ANKRD16, C8 orf16, ATF71P2, PRIC285 |
| Prostate cancer | Serum/saliva | Prostate specific antigen (PSA) |
| | Urine | PCA3, GOLPH2, SPINK1, TMPRSS2:ERG |
| Infections | | See Table 2 |
| Dental | Saliva | aspartate aminotransferase (AST) and |
| caries/periodontal | | alkaline phosphatase (ALP), uric acid and |
| disease | | albumin; 12-HETE; MMP-8, TIMP-1, and |
| | | ICTP |
| Heavy metal | saliva | lead, cadmium |
| poisoning | | |
| Drugs/drug | saliva | marijuana, Cocaine (crystalline tropane |
| metabolites | | alkaloid), methamphetamine, amphetamine, |
| | | heroin, methyltestosterone, mesterolone, |
| | | morphine, cyclophosphamide metabolites, |
| | | Haloperidol, barbiturates; antipyrine, |
| | | caffeine, cisplatin, cyclosporine, diazepam, |
| | | digoxin, methadone, phenytoin, theophylline, |
| | | tolbutamide. Nicotine/cotinine, cannabis |
| | urine | trichloroethanol glucuronide, Anabolic |
| | | steroids, Androstenedione, Benzodiazepines, |
| | | Chlordiazepoxide, Lorazepam, Zidovudine |
| Allergies | saliva | Allergen-specific IgAs (see Tables 7 and 9) |

In some instances, the biomarker that can be detected by the present method is an antibody in a sample, e.g., a diagnostic sample, that is probative for diagnosing a disease or health condition of the subject from which the sample is derived. A signal-amplifying nanosensor configured to detect an antibody analyte may contain an antibody epitope to which the antibody analyte specifically binds as a capture agent. In some cases, the disease or health condition is related to an autoimmune disease, in which antibodies against its own body (autoantibodies) induce an autoimmune response. In some embodiments, the antibody analyte of interest is an IgA, IgM, IgE, IgD, or IgG antibody. In some instances, a labeling agent may contain a moiety that binds specifically to regions of an antibody analyte that is specific to the particular type of antibody. For example, a labeling agent containing peptide M, SSL7 or Jacalin may bind specifically to IgA, and a labeling agent containing Protein G may bind specifically to IgG. Protein L may be used to bind to all types of antibodies.

Tables 4 provides a list of autoantibody targets, which can be used, in whole or as an epitope fragment, as a capture agent in the present method to measure the amount of the epitope-binding antibody analyte in a sample and thereby diagnose the associated disease or health condition, e.g., an autoimmune disease. In some cases, the disease or health condition is related to an immune response to an allergen. Table 5 provides a list of allergens, which can be used, in whole or as an epitope fragment, as a capture agent in the present method to measure the amount of the epitope-binding antibody analyte in a sample and thereby diagnose the associated disease or health condition, e.g., an allergy. In certain instances, the disease or health condition is related to an infectious disease, where the infectious agent may be diagnosed based on information including the measured amount of antibodies against one or more epitopes derived from the infectious agent (e.g., lipopolysaccharides, toxins, proteins, etc). Tables 6 provides a list of infectious-agent derived epitopes which can be used, in whole or as an epitope fragment, as a capture agent in the present method to measure the amount of the epitope-binding antibody analyte in a sample and thereby diagnose the associated disease or health condition, e.g., an infection. Other epitopes or antigens that may be suitable for use in the present diagnostic method are described in, e.g., PCT App. Pub. No. WO 2013164476, which is incorporated herein by reference. It will also be clear to one with ordinary skill in the art that the subject signal-amplifying nanosensors may be configured to capture and detect many more antibody analytes that that are diagnostic of a disease or health condition. The signal-amplifying nanosensor may be configured so that epitopes present on the signal-amplifying nanosensor are not cross-reactive, i.e., are bound by antibodies that bind non-specifically to many epitopes present on the signal-amplifying nanosensor.

TABLE 4
Diagnostic Autoantibody Epitopes

| Disease/condition | Autoantibody Targets |
|---|---|
| Cancer | ACAA2; ANXA13; AQP2; ASPA; BCL2; BCL2L1; BIK; |
| | CD160; CD37; CDK4; CDK6; CHEK2; CITED2; CNN2; |
| | CTSC; CTSZ; CycE2; ELK1; FGF10; FN1; GATA3; GJA1; |
| | GNRH1; GRB2, HBB; HBE1; HIST2H2AA; HPRT1; ID2; |
| | IER2; IFI27; IFITM1; IFITM2; IL15; IL18; IL8; IL9; KRT16; |
| | LALBA; LDHA; LDHB; LECT1; MAFK; Mage3; MAGEA3; |
| | MMP2; NPPB; OAS1, p21; p53; PCNA; PENK; PEX3; PHB; |
| | PHYH; PI3; PKBα; PLN; S100A7; SCAMP1; SCGB1A1; |
| | SLC38A5; SNRP2; SNX9; SST; SSTR2; TACSTD1; TNNC2; |
| | TOB1; TSG101; VDRIP; WNT2, p62 and Koc; ZFP161, |
| | Ubiquilin-1, HOX-B6, YB-1, Osteonectin, ILF3 |
| Squamous cell lung carcinoma | protein kinase C and p53-binding protein (TP53 BP), |
| | lymphoid blast crisis oncogene (LBC), |
| Small cell lung cancer | SOX families B1 and B2, MUC-1, |
| Lung cancer | MUC-1, p53, surviving, LAMR1, annexin I, 14-3-3-theta; |
| | AKR1B10; GOT2; HNRPR; PDIA3; NME2; RTN4; HI1FX; |
| | G3BP; HSPCA; ACTN4; PGP9.5; |
| Colorectal cancer | MUC-1, surviving, p-53; translationally controlled tumor |
| | protein; HSPC218; Ribosomal protein S18; v-Fte-1; v-Fos |
| | transformation effector protein; MAGEA3, SSX2, NY-ESO-1, |
| | HDAC5, MBD2, TRIP4, NY-CO-45, KNSL6, HIP1R, Seb4D, |
| | KIAA1416, and LMNA; UCHL3 |
| Hepatocellular carcinoma | fibrillarin and p330d/CENP-F, insulin-like growth factor II |
| | mRNA-binding proteins (IMP) 1, IMP3 and p53, NOR-90, |
| | nucleophosmin/protein B23, cyclin B1, DNA topoisomerase II |
| | (topo II), p62, HCC1, SG2NA, MAGE-C2, AF146731; |
| | AF219119; AF146019; Ligatin; AF220416; AF218421; |
| | AF257175; AF244135; AF243495; AF287265; AF258340; |
| | AF270491; AF286340; small nuclear RNA-associated sm-like |
| | protein; Dna J protein; CENP-F; translationally controlled |
| | tumor protein; LDH-A; Albumin; Hsp89αΔN; SEC63; |
| | AF100141; 14.5 kDa protein; GCF2; Metallopanstimulin 1; |
| | SMP-30 D31815; Cg1 protein,; C3VS protein; F1-ATPase, β |
| | subunit; Human ribosomal protein L10; Pre-apolipoprotein |
| | CIII; Galactose-1-phosphate-uridyl-transferase (GALT); |
| | DNA polymerase Δ, small subunit; Mitochondrial DNA |
| Renal cancer | AF257175; small nuclear RNA-associated sm-like protein; |
| | Dna J protein; smooth muscle protein 22-alpha (SM22-alpha); |
| | carbonic anhydrase I (CAI) |
| Acute leukemia | Rho GDP dissociation inhibitor 2, γ-actin, F-actin capping |
| | protein (CAPZA1), heterogeneous nuclear ribonucleoprotein L |
| | (hnRNP L), tubulin-α 6, PCNA |
| Chronic lymphocytic leukemia | KIAA1641; PIPMT; FosB; ZNF268; SEBD4; Ikaros; |
| | p75/LDEGF; CHIP; PYGB; ZNF148; KIAA0336; RPL11; |
| | FMNL; HGRG8 |
| non-Hodgkin's lymphoma | CENP-F, |
| Multiple myeloma | NY-ESO-1 |
| melanoma | NY-ESO-1, MAGE-1, BAGE, GAGE, MART-1/melan A, |
| | gp100, and tyrosinase |
| Pancreatic cancer | Calreticulin, DEAD-box protein 48 (DDX48) |
| Ovarian cancer | ACSBG1, AFP, CSNK1A1 L, DHFR, MBNL1, TP53, PRL, |
| | PSMC1, PTGFR, PTPRA, RAB7L1, and SCYL3, her2/neu, |
| | MUC1, c-myc, ECPKA, and NY-ESO-1, p53, UBQLN1, |
| | HOXB6, TOP2A, putative helicase-RUVBL (RUVBL), |
| | HMBA-inducible (HEXIM1), DDX5 and HDCMA |
| Prostate cancer | Bcl2, NY-ESO-1, survival protein lens epithelium-derived |
| | growth factor p75 (LEDGF/p75), PRDX6/AOP2, clusterin, |
| | DJ-1, superoxide dismutase, alcohol dehydrogenase, HSP70, |
| | HSP27/HSPB1, lactoylglutathione lyase, glucose-regulated |
| | protein-78 kDa (GRP78), p62, Koc, and IMP1, α-Methylacyl- |
| | coenzyme A racemase and 5-α-reductase, AKRIA1; Brd2; |
| | C17 orf 25; CAPZA1; c-MYC; Cyclin A; Cyclin B1; Cyclin |
| | D1; Drebrin; eIF4G1; HIP1; HSPA8; Lactoylglutathione |
| | lyase; MAD-CT-1; MAD-CT-2; No55; P53; P62; P90; PP4R; |
| | PIP; PSA; RPL13a; RPL22; Survivin; Syntenin 1; TDP-43; |
| | VCP; vWF; Lage-1, and Xage-1; bromo domain-containing |
| | protein 2 (BRD2), ribosomal proteins L22 and L13a, |
| | XP_373908 |
| Breast cancer | p53, c-myc, NY-ESO-1, BRCA1, BRCA2, HER2, MUC1, |
| | IGFBP-2, TOPO2α, ribosomal protein S6, eukaryotic |
| | elongation factor 2, eukaryotic elongation factor 2 kinase, and |
| | heat shock protein 90 (HSP90), Ku protein, topoisomerase I, |
| | and the 32-kDa subunit of replication protein A; CENP-F; |
| | AF146731; int-2, pentraxin I, integrin beta5, cathepsin L2 and |
| | S3 ribosomal protein; RNA-binding protein regulatory subunit |
| | (RS), DJ-1 oncogene, glucose-6-phosphate dehydrogenase, |
| | heat shock 70-kDa protein 1 (HS71), and dihydrolipoamide |
| | dehydrogenase |
| Nasopharyngeal carcinoma | MAGE, HSP70, Fibronectin, CD44, EBV antigens |
| Oral cancer | Cyclin B1, p53 |
| Oral squamous cell carcinoma | p53 |
| Head and neck squamous cell | CASP-8, SART-1, TREX1, 3′ repair exonuclease; BRAP |
| carcinoma | (BRCA1 associated): Nuclear localization protein; Trim 26 |
| | zinc finger domains; GTF21 transcription factor. Murine |
| | homolog TF11-1; NSEP1 (YB-1) transcription factor; MAZ |
| | transcription factor associated with c-myc; SON (DBP-5; |
| | KIAA1019; NREBP DNA binding protein); NACA nascent |
| | polypeptide-associated complex; NUBP2 nucleotide binding |
| | protein; EEF2 Translation elongation factor 2; GU2 Putative |
| | RNA helicase; RPLI3A ribosomal protein; SFRS21P |
| | (CASP11; SIP1; SRRP1290 splicing factor); RPS12 |
| | ribosomal protein; MGC2835 RNA helicase; TMF1, TATA |
| | modulatory factor; PRC1 regulator of cytokinesis; KRT14 |
| | keratin 14; Viniculin; H2AFY histone family member; SLK |
| | (KIAA02304) Ste related kinase; NOL3 (ARC) nuclear |
| | protein 3, apoptosis repressor; DNAJA2 member of Hsp40 |
| | family; DNAJA1 member of HSP40 family; LINE-1 |
| | retrotransposon; MOG (HSPC 165) Homolog of yeast |
| | protein; LIMS1 (PINCH): LIM and senescent antigen-like |
| | domain; COPB2 coatomer protein complex subunit protein; |
| | FLJ22548 hypothetical protein; C21orf97; FLJ21324; |
| | MGC15873; SSNA1 Sjogrens syndrome nuclear autoantigen |
| | 1; KIAA0530, zinc finger domain; rat stannin; hypothetical |
| | protein DKFZp4340032; human FLJ23089; PC326 |
| Esophageal cancer | NY-ESO-1; SURF1, HOOK2, CENP-F, ZIC2, hCLA-iso, Ki- |
| | 1/57, enigma, HCA25a, SPK, LOC146223 and |
| | AGENCOURT_7565913 |
| Metabolic syndrome/prediabetes | GFAP |
| Diabetes | Zn transporter 8, glutamic acid decarboxylase (GAD), CD38, |
| | gad65, IA2, insulin, MRPS31, ICA1, L-type voltage gated |
| | calcium channel; SNRPB2; DDX42; C11orf63; TCOF1; |
| | TSSK2; KDM4B; PDGFB; LTK; RPL14; VIM; GTF2I; |
| | BCL2L13; LARP6; DKFZP434K028; USP39; SERBP1; |
| | CCL19; GAD2; MCM10; ZNF688; PTEN; RP6-166C19.11; |
| | GIPC1; TIGD1; CCDC131; HTF9C; SOX5; MCF2L; |
| | TRAF3IP1; 6CKINE; ACY3; AMMECR1L; ARHGAP9; |
| | ASNS; BATF2; BMX; C9ORF25; CDC2; CHGB; CXORF38; |
| | CXORF56; DMD; ECHDC1; EIF3F; EPHA2; ERMN; |
| | FAM136A; (includes; EG: 84908); FILIP1; FLT1; GART; |
| | GIMAP6; GNG7; GTF2F1; HGS; IFI6; KDM4B; LACE1; |
| | LGALS1; LGALS7; LIMS2; LTK; LUC7L; NCAPG; |
| | (includes; EG: 64151); NME6; NUPL1; PAK4; PDE4DIP; |
| | PSIP1; RAB20; RNGTT; RPS3; SPG20; TALDO1; TBRG1; |
| | THAP1; TRAF3IP2; UBL4A; ZC3HC1; ZNF131; |
| | RAD51AP1; HADH; (HADH); C11orf16; (C11orf16); TAC3; |
| | ABR; ECE1; PPP1R2; GRINL1A; ABR; C19orf44; |
| | MUSTN1; ETHE1; BMI1; BAZ2B; ; TBC1D22A; |
| | CAMK2N2; ASS1; CCNY; MARK2; RAD51AP1; RAB38; |
| | RIOK1; HSP90AA1; C11orf74; ARID3A; LMOD1; |
| | CAPRIN1; ITGB3BP; MND1; SGK; NADK; MED9; LDHA; |
| | ARHGAP26; ANKRA2; CRY2; IL23A; DUSP14; ZBTB44; |
| | SIRT1; SLC2A3; GPR172B; CCDC89; BATF; HMOX1; |
| | ARRDC1; USF2; GBGT1; EDC3; SGIP1; GCGR; ZRANB2; |
| | NLGN4Y; GJB6; CDK10; PSG1; CCDC74A; DENND1C; |
| | MAP2K6 |
| Autoimmune heart disease | cardiac troponin I (cTnI) |
| Immunoglobulin A nephropathy | PRKD1, MATN2, DDX17, UBE2W, CDKN1 B, SOD2, |
| | FLOT2, IQCK, BLZF1, BRD9, CDS2, EFNA3, EIF4A2, |
| | FLU, LIMCH1, MAGEA4, MEF2D, MLLT6, MRPL28, |
| | MUTED, NKAIN4, PCTK1, PLXNA1, PODN, POLH, |
| | PRKD2, RNF1 1 3A, SEPT5, TNS1, TOM1, TRPV4, |
| | USP12, ZMYM3, CIAPIN1, GDI2, HSPA8, SERPINA5 and |
| | TGM1 |
| End stage renal disease | IGLC1; IGHG1; EDC3; IGHG1; APEX2; CD3D; TRIM21; |
| | IGKV1-5; IGHG3; CTLA-FC; CD7; CLIP4; MAPRE1; |
| | SNRPB2; IGHG1; ZBTB44; CD3D; IGHG1; TRAM1; ERR |
| | beta-; LBD; CNBP; OLFM1; IGHM; SIRT5; CEP290; |
| | PHLDA1 |
| Glomerular nephritis | |
| Addison's disease | 21-hydroxylase, P450-17α-hydroxylase (17OH) and P450-side |
| | chain cleavage (SCC) |
| Primary ovarian insufficiency | Jo-1, proteinase 3 (PR3) |
| Sjögren's syndrome | IgA, IgG, IgM autoantibodies; IgA, lactoferrin and beta2- |
| | microglobulin; lysozyme C, and cystatin C, amylase and |
| | carbonic anhydrase |
| | SSA/Ro; LA/SS-B |
| Systemic lupus erythematosus | CDC25B, APOBEC3G, ARAF, BCL2A1, CLK1, CREB1, |
| (SLE) | CSNK1G1, CSNK2A1, CWC27, DLX4, DPPA2, EFHD2, |
| | EGR2, ERCC2, EWSR1, EZH2, FES, FOS, FTHL17, GEM, |
| | GNA15, GNG4, HMGB2, HNRNPUL1, HOXB6, ID2, IFI35, |
| | IGF2BP3, IGHG1, JUNB, KLF6, LGALS7, LIN28A, |
| | MLLT3, NFIL3, NRBF2, PABPC1, PATZ1, PCGF2, |
| | PPP2CB, PPP3CC, PRM1, PTK2, PTPN4, PYGB, RET, |
| | RPL18A, RPS7, RRAS, SCEL, SH2B1, SMAD2, STAM, |
| | TAF9, TIE1, UBA3, VAV1, WT1, ZAP70, or ZNRD1 |
| | KIT, C6orf93, RPL34, DOM3Z, COPG2, DNCL12, RRP41; |
| | FBXO9; RALBP1, PIAS2; EEF1D; CONI; KATNB1; |
| | POLR2E; CCT3; KIAA0643; RPL37A, GTF2H2; |
| | MAP2K5; CDK3; RPS6KA1; MARK4, MTO1; |
| | MGC42105; NFE2L2; WDR45L, STK4, PFKFB3; |
| | NTRK3; MLF1; TRIM37, ACTL7B, RPL18A, CKS1B; |
| | TUBA1, NME6, SUCLA2, IGHG1, PRKCBP1; BAG3; |
| | TCEB3; RPL15, SSX4; MAP2K7; EEF1G; RNF38, |
| | PHLDA2, KCMF1; NUBP2, VPS45A |
| | SSA/Ro; dsDNA; Smith; histones; thrombin; v-Fos |
| | transformation effector protein, tryptase, Sm antigen, beta 2; |
| | cardiolipin; glycoprotein I β2; Endothelial PC/activated PC |
| | receptor; human gamma enolase |
| CREST syndrome | centromere |
| Systemic sclerosis | Type I topoisomerase |
| Primary biliary cirrhosis | nucleoporin 62, Sp100 nuclear antigen, nucleoporin 210 kDa, |
| | mitochondria, mitochondrial pyruvate dehydrogenase (PDH) |
| | or E3 binding protein |
| Dermatitis herpetiformis | eTG |
| Miller-Fisher Syndrome | ganglioside GQ1B |
| Wegener's granulomatosis | c-ANCA |
| Neuropathies | ganglioside GD3, ganglioside GM1, GA1, GM2, MAG |
| microscopic polyangiitis | p-ANCA |
| Polymyositis | Signal recognition particles |
| scleromyositis | exosome complex Signal recognition particles |
| myasthenia gravis | nicotinic acetylcholine receptor Signal recognition particles, |
| | muscle-specific kinase (MUSK) Signal recognition particles |
| Lambert-Eaton myasthenic | voltage-gated calcium channel (P/Q-type) |
| syndrome | |
| Hashimoto's thyroiditis | thyroid peroxidase |
| Graves' disease | TSH receptor |
| paraneoplastic cerebellar | Hu, Yo (cerebellar Purkinje Cells), amphiphysin |
| syndrome | |
| encephalitis | voltage-gated potassium channel (VGKC), N-methyl-D- |
| | aspartate receptor (NMDA) |
| Sydenham's chorea | basal ganglia neurons |
| antiphospholipid syndrome | glycoprotein 1 (2GP1), Endothelial PC/activated PC receptor |
| Systemic vasculitis | proteinase 3 (PR3) and myeloperoxidase (MPO) |
| Neuromyelitis | aquaporin-4 |
| Allergies | Allergen-specific IgAs |
| Rheumatoid arthritis | Rheumatoid factor, cyclic citrullinated protein; human |
| | cartilage gp39 peptides and type II collagen; citrullinated |
| | fibrinogen, citrullinated vimentin, citrulline-substituted |
| | filaggrin peptides, hnRNP-A2/B1, BiP, tryptase |
| Asthma | tryptase |
| Multiple sclerosis | myelin basic protein, spectrin, fodrin, myelin oligodentrocyte |
| | glycoprotein, proteolipid protein (PLP), 2′,3′-cyclic |
| | nucleotide-phosphodiesterase (CNP), Glc(α1,4)Glc(α) |
| | (GAGA4), Glc(α1,6)Glc(α) (GAGA6) |
| amyotrophic lateral sclerosis | HMGB1 |
| (ALS) | |
| Idiopathic thrombocytopenic | platelet glycoprotein (GP) IIb/IIIa, GPIb/IX, GPIa/IIa |
| purpura | |
| Thrombosis | thrombomodulin |
| Cardiovascular disease | Endothelial PC/activated PC receptor; IL-1 alpha, alpha- |
| | actinin-2 (aActn2); alpha-Myosin Heavy Chain (alpha-MHC-S |
| | 1); SI fragment of alpha-Myosin Heavy Chain 6 (alpha- |
| | MHC6-S1); alpha-Myosin Heavy Chain 7 (MyHC7) |
| post-streptococcal disease such | ELAVL2, ELAVL3, ELAVL4, Nova-1, Nova-2, Cdr1, Cdr2; |
| as PANDAS, post-GABHS | and Cdr3 |
| glomerulonephritis, rheumatic | |
| fever, autism and Syndenham's | |
| chorea | |
| Parkinson's Disease | alpha-synuclein; myelin basic protein (MBP), proteolipid |
| | protein (PLP), myelin oligodendrocyte glycoprotein (MOG), |
| | myelin associated glycoprotein (MAG), oligodendrocytes |
| | specific protein (OSP) |
| pernicious anemia | Vitamin B₁₂ |

TABLE 5
Allergen Epitopes

| Source | Allergen |
|---|---|
| mites | Acas13, Blot1, Blot3, Blot4, Blot5, Blot6, Blot10, Blot11, Blot12, |
| | Blot13, Blot19; American house dust mite (Derf1, Derf2, Derf3, |
| | Derf7, Derf10, Derf11, Derf14, Derf15, Derf16, Derf17, Derf18w); |
| | house dust mite (Derm1); European house dust mite (Derp1, Derp2, |
| | Derp3, Derp4, Derp5, Derp6, Derp7, Derp8, Derp9, Derp10, Derp11, |
| | Derp14, Derp20, Derp21); mite (Eurm2; Eurm14); storage mite |
| | (Glyd2, Lepd2, Lepd5, Lepd7, Lepd10, Lepd13, Tyrp2, Tyrp13); |
| | Dermatophagoides farinae (Derf1.0101, Derf1.0102, Derf1.0103, |
| | Derf1.0104, Derf1.0105, Derf2.0101, Derf2.0102, Derf2.0103, |
| | Derf2.0104, Derf2.0105, Derf2.0106, Derf2.0107, Derf2.0108, |
| | Derf2.0109, Derf2.0110, Derf2.0111, Derf2.0112, Derf2.0113, |
| | Derf2.0114, Derf2.0115, Derf2.0116, Derf2.0117); Dermatophagoides |
| | pteronyssinus (Derp1.0101, Derp1.0102, Derp1.0103, Derp1.0104, |
| | Derp1.0105, Derp1.0106, Derp1.0107, Derp1.0108, Derp1.0109, |
| | Derp1.0110, Derp1.0111, Derp1.0112, Derp1.0113, Derp1.0114, |
| | Derp1.0115, Derp1.0116, Derp1.0117, Derp1.0118, Derp1.0119, |
| | Derp1.0120, Derp1.0121, Derp1.0122, Derp1.0123, Derp2.0101, |
| | Derp2.0102, Derp2.0103, Derp2.0104, Derp2.0105, Derp2.0106, |
| | Derp2.0107, Derp2.0108, Derp2.0109, Derp2.0110, Derp2.0111, |
| | Derp2.0112, Derp2.0113); Euroglyphus maynei (Eurm2.0101, |
| | Eurm2.0102); Glycyphagus domesticus (Glyd2.0101, Glyd2.0201); |
| | and Lepidoglyphus destructor (Lepd2.0101, Lepd2.0101, Lepd2.0101, |
| | Lepd2.0102, Lepd2.0201, Lepd2.0202) |
| Pollen | Short Ragweed (Ambrosia artemisiifolia) allergen, Amb a 1, Amba2, |
| | Amba3, Amba5, Amba6, Amba7, Amba8, Amba9, Amba10; Betula |
| | verrucosa allergen, Bet v 1, Phleum pratense allergen, Phl p 5), giant |
| | ragweed (Ambt5); mugwort (Artv1, Artv2, Artv3, Artv4, Artv5, |
| | Artv6); sunflower (Hela1, Hela2, Hela3); Mercurialis annua (Mera1); |
| | lamb's-quarters, pigweed (Chea1); white goosefoot (Chea2, Chea3); |
| | Russian-thistle (Salk1); Rosy periwinkle (Catr1); English plantain |
| | (Plal1); Japanese hop (Humj1); Parietaria judaica (Parj1, Parj2, Parj3); |
| | Parietaria officinalis (Parol); Ambrosia artemisiifolia (Amba8.0101, |
| | Amba8.0102, Amba9.0101, Amba9.0102); Plantago lanceolata |
| | (Plal1.0101, Plal1.0102, Plal1.0103); and Parietaria judaica |
| | (Parj1.0101, Parj1.0102, Parj1.0201, Par2.0101, Parj2.0102, |
| | Parj3.0101, Parj3.0102), Bermuda grass (Cynd1, Cynd7, Cynd12, |
| | Cynd15, Cynd22w, Cynd23, Cynd24); orchard grass (Dacg1, Dacg2, |
| | Dacg3, Dacg5); meadow fescue (Fesp4w); velvet grass (Holl1); rye |
| | grass (Lolp1, Lolp2, Lolp3, Lolp5, Lolp11); canary grass (Phaa1); |
| | Timothy (Phlp1, Phlp2, Phlp4, Phlp5, Phlp6, Phlp11, Phlp12, Phlp13); |
| | Kentucky blue grass (Poap1, Poap5); Johnson grass (Sorh1); Cynodon |
| | dactylon (Cynd1.0101, Cynd1.0102, Cynd1.0103, Cynd1.0104, |
| | Cynd1.0105, Cynd1.0106, Cynd1.0107, Cynd1.0201, Cynd1.0202, |
| | Cynd1.0203, Cynd1.0204); Holcus lanatus (Holl1.0101, Holl1.0102); |
| | Lolium perenne (Lolp1.0101, Lolp1.0102, Lolp1.0103, Lolp5.0101, |
| | Lolp5.0102); Phleum pretense (Phlp1.0101, Phlp1.0102, Phlp4.0101, |
| | Phlp4.0201, Phlp5.0101, Phlp5.0102, Phlp5.0103, Phlp5.0104, |
| | Phlp5.0105, Phlp5.0106, Phlp5.0107, Phlp5.0108, Phlp5.0201, |
| | Phlp5.0202); and Secale cereale (Secc20.0101, Secc20.0201), Alder |
| | (Alng1); Birch (Betv1, Betv2, Betv3, Betv4, Betv6, Betv7); hornbeam |
| | (Carb1); chestnut (Cass1, Cass5, Cass8); hazel (Cora1, Cora2, Cora8, |
| | Cora9, Cora10, Cora11); White oak (Quea1); Ash (Frae1); privet |
| | (Ligv1); olive (Olee1, Olee2, Olee3, Olee4, Olee5, Olee6, Olee7, |
| | Olee8, Olee9, Olee10); Lilac (Syrv1); Sugi (Cryj1, Cryj2); cypress |
| | (Cupa1); common cypress (Cups1, Cups3w); mountain cedar (Juna1, |
| | Juna2, Juna3); prickly juniper (Juno4); mountain cedar (Juns1); |
| | eastern red cedar (Junv1); London plane tree (Plaa1, Plaa2, Plaa3); |
| | date palm (Phod2); Betula verrucosa (Betv1.0101, Betv1.0102, |
| | Betv1.0103, Betv1.0201, Betv1.0301, Betv1.0401, Betv1.0402, |
| | Betv1.0501, Betv1.0601, Betv1.0602, Betv1.0701, Betv1.0801, |
| | Betv1.0901, Betv1.1001, Betv1.1101, Betv1.1201, Betv1.1301, |
| | Betv1.1401, Betv1.1402, Betv1.1501, Betv1.1502, Betv1.1601, |
| | Betv1.1701, Betv1.1801, Betv1.1901, Betv1.2001, Betv1.2101, |
| | Betv1.2201, Betv1.2301, Betv1.2401, Betv1.2501, Betv1.2601, |
| | Betv1.2701, Betv1.2801, Betv1.2901, Betv1.3001, Betv1.3101, |
| | Betv6.0101, Betv6.0102); Carpinus betulus (Carb1.0101, Carb1.0102, |
| | Carb1.0103, Carb1.0104, Carb1.0105, Carb1.0106, Carb1.0106, |
| | Carb1.0106, Carb1.0106, Carb1.0107, Carb1.0107, Carb1.0108, |
| | Carb1.0201, Carb1.0301, Carb1.0302); Corylus avellana (Cora1.0101, |
| | Cora1.0102, Cora1.0103, Cora1.0104, Cora1.0201, Cora1.0301, |
| | Cora1.0401, Cora1.0402, Cora1.0403, Cora1.0404); Ligustrum |
| | vulgare (Ligv1.0101, Ligv1.01.02); Olea europea (Olee1.0101, |
| | Olee1.0102, Olee1.0103, Olee1.0104, Olee1.0105, Olee1.0106, |
| | Olee1.0107); Syringa vulgaris (Syrv1.0101, Syrv1.0102, Syrv1.0103); |
| | Cryptomeria japonica (Cryj2.0101, Cryj2.0102); and Cupressus |
| | sempervirens (Cups1.0101, Cups1.0102, Cups1.0103, Cups1.0104, |
| | Cups1.0105) |
| mold | Alternaria alternata allergen, Alt a 1, Alta3, Alta4, Alta5, Alta6, |
| | Alta7, Alta8, Alta10, Alta12, Alta13, Aspergillus fumigatus allergen, |
| | Asp f 1, Aspf2, Aspf3, Aspf4, Aspf5, Aspf6, Aspf7, Aspf8, Aspf9, |
| | Aspf10, Aspf11, Aspf12, Aspf13, Aspf15, Aspf16, Aspf17, Aspf18, |
| | Aspf22w, Aspf23, Aspf27, Aspf28, Aspf29); Aspergillus niger |
| | (Aspn14, Aspn18, Aspn25); Aspergillus oryzae (Aspo13, Aspo21); |
| | Penicillium brevicompactum (Penb13, Penb26); Penicillium |
| | chrysogenum (Pench13, Pench18, Pench20); Penicillium citrinum |
| | (Penc3, Penc13, Penc19, Penc22w, Penc24); Penicillium oxalicum |
| | (Peno18); Fusarium culmorum (Fusc1, Fusc2); Trichophyton rubrum |
| | (Trir2, Trir4); Trichophyton tonsurans (Trit1, Trit4); Candida albicans |
| | (Canda1, Canda3); Candida boidinii (Candb2); Psilocybe cubensis |
| | (Psic1, Psic2); shaggy cap (Copc1, Copc2, Copc3, Copc5, Copc7); |
| | Rhodotorula mucilaginosa (Rhom1, Rhom2); Malassezia furfur |
| | (Malaf2, Malaf3, Malaf4); Malassezia sympodialis (Malas1, Malas5, |
| | Malas6, Malas7, Malas8, Malas9, Malas10, Malas11, Malas12, |
| | Malas13); Epicoccum purpurascens (Epip1); and Alternaria alternate |
| | (Alta1.0101, Alta1.0102), Aspergillus versicolor antigen, S. chartarum |
| | antigen), Cladosporium herbarum (Clah2, Clah5, Clah6, Clah7, Clah8, |
| | Clah9, Clah10, Clah12); Aspergillus flavus (Aspf113); |
| mammals | Bos domesticus dander allergen, Bos d 2, Bosd3, Bosd4, Bosd5, |
| | Bosd6, Bosd7, Bosd8, Bosd2.0101, Bosd2.0102, Bosd2.0103, Canis |
| | familiaris allergen, Can f 1, Canf2, Canf3, Canf4, Equus |
| | caballus allergen, Equc1, Equc2, Equc3, Equc4, Equc5, Felis |
| | domesticus allergen, Fel d 1, Feld2, Feld3, Feld4, Feld5w, Feld6w, |
| | Feld7w, guinea pig (Cavp1, Cavp2); Mouse Urinary Protein (MUP, |
| | Musm1) allergen, Mus m 1, Rat Urinary Protein (RUP, Ratn1) |
| | allergen, Ratn 1., Equus caballus (Equc2.0101, Equc2.0102)) |
| Insects | Mosquito (Aeda1, Aeda2); honey bee (Apim1, Apim2, Apim4, |
| | Apim6, Apim7); bumble bee (Bomp1, Bomp4); German cockroach |
| | (Blag1, Blag2, Blag4, Blag5, Blag6, Blag7, Blag8); American |
| | cockroach (Pera1, Pera3, Pera6, Pera7); midge (Chit1-9, Chit1.01, |
| | Chit1.02, Chit2.0101, Chit2.0102, Chit3, Chit4, Chit5, Chit6.01, |
| | Chit6.02, Chit7, Chit8, Chit9); cat flea (Ctef1, Ctef2, Ctef3); pine |
| | processionary moth (Thap1); silverfish (Leps1); white face hornet |
| | (Dolm1, Dolm2, Dolm5); yellow hornet (Dola5); wasp (Pola1, Pola2, |
| | Pola5, Pole1, Pole5, Polf5, Polg5, Polm5, Vesvi5); Mediterranean |
| | paper wasp (Poldi, Pold4, Pold5); European hornet (Vespc1, Vespc5); |
| | giant asian hornet (Vespm1, Vespm5); yellowjacket (Vesf5, Vesg5, |
| | Vesm1, Vesm2, Vesm5, Vesp5, Vess5, Vesv1, Vesv2, Vesv5); |
| | Australian jumper ant (Myrp1, Myrp2); tropical fire ant (Solg2, |
| | Solg4); fire ant (Soli2, Soli3, Soli4); Brazilian fire ant (Sols2); |
| | California kissing bug (Triap1); Blattella germanica (Blag1.0101, |
| | Blag1.0102, Blag1.0103, Blag1.02, Blag6.0101, Blag6.0201, |
| | Blag6.0301); Periplaneta Americana (Pera1.0101, Pera1.0102, |
| | Pera1.0103, Pera1.0104, Pera1.02, Pera3.01, Pera3.0201, Pera3.0202, |
| | Pera3.0203, Pera7.0101, Pera7.0102); Vespa crabo (Vespc5.0101, |
| | Vespc5.0101); and Vespa mandarina (Vesp m 1.01, Vesp m 1.02) |
| Rubber | rubber (latex)(Hevb1, Hevb2, Hevb3, Hevb4, Hevb5, Hevb6.01, |
| | Hevb6.02, Hevb6.03, Hevb7.01, Hevb7.02, Hevb8, Hevb9, Hevb10, |
| | Hevb11, Hevb12, Hevb13); Hevea brasiliensis (Hevb6.01, |
| | Hevb6.0201, Hevb6.0202, Hevb6.03, Hevb8.0101, Hevb8.0102, |
| | Hevb8.0201, Hevb8.0202, Hevb8.0203, Hevb8.0204, Hevb10.0101, |
| | Hevb10.0102, Hevb10.0103, Hevb11.0101, Hevb11.0102) |
| Others | Nematode (Anis1, Anis2, Anis3, Anis4); pigeon tick (Argr1); worm |
| | (Ascs1); papaya (Carp1); soft coral (Denn1); human autoallergens |
| | (Homs1, Homs2, Homs3, Homs4, Homs5); obeche (Trips1) |

TABLE 6
Infectious Agent-derived Epitopes

| Infectious Agent | Epitope |
|---|---|
| Mycobacterium tuberculosis | isocitrate dehydrogenase (ICDs) |
| Influenza virus | Hemagglutinin (H1), neuraminidase (N1) |
| Dengue virus | envelope (E) |
| Toxoplasma gondii | microneme proteins, SAG1, SAG2, GRA1, GRA2, |
| | GRA4, GRA6, GRA7, GRA3, ROP1, ROP2, p30, MIC3, |
| | MIC2, M2AP, p29, p35, p66 |
| Entamoeba histolytica | M17, neutral thiol proteinase |
| Streptococcus pneumonia | Pneumolysin, pneumococcal histidine triad D (PhtD), |
| | pneumococcal choline-binding protein A (PepA), |
| | pneumococcal histidine triad E (PhtE), LytB |
| Mycoplasma pneumonia | exotoxin |
| Epstein-Barr virus | VCA |
| Helicobacter pylori | CagA, Vacuolating protein, ureB, hsp60, ureH, urea, |
| | ferritin like protein |
| Campylobacter jejuni | PEB1, PEB3 |
| Bacillus anthracis | SAP |
| SARS virus | RNA-dependent replicases Ia and Ib, spike (S) protein, |
| | small envelope (E) protein, membrane (M) protein, and |
| | nucleocapsid (N) protein |
| Ebola virus | Nucleoprotein N |
| Schmallenberg virus | N nucleoprotein |
| enterovirus 71 | VPl protein |
| Japanese Encephalitis virus | soluble E protein, envelope E protein |
| Ross River virus | soluble E2 protein |
| Mayaro virus | soluble E2 protein |
| Equine Encephalitis viruses | soluble E2 protein |
| Akabane virus | N nucleoprotein |
| human betacoronavirus | Nucleoprotein N, protein S |
| Hepatitis C virus | protein C, core antigen |
| Hepatitis E virus | protein C |
| Plasmodium falciparum | MSP-1 + AMA-1 protein |
| Leptospira interrogans | HbpA, LruA, LruB, or LipL32 |

In some instances, the biomarker to be detected using the present method is a micro RNA (miRNA) biomarker that is associated with a disease or a health condition. The following Table 7 provides a list of miRNA biomarker that can be detected using the present signal-amplifying nanosensor (when used in conjunction with an appropriate complementary nucleic acid, or other capture agent), and their associated diseases/health conditions.

TABLE 7
Diagnostic miRNA Markers

| Disease/Condition | Marker* |
|---|---|
| Breast cancer | miR-10b, miR-21, miR-125b, miR-145, miR-155, miR-191, miR- |
| | 382, MiR-1, miR-133a, miR-133b, miR-202, miR-1255a, miR-671- |
| | 3p, miR-1827, miR-222, miR-744, miR-4306, miR-151-3p, miR-130, |
| | miR-149, miR-652, miR-320d, miR-18a, miR-181a, miR-3136, miR- |
| | 629, miR-195, miR-122, miR-375, miR-184, miR-1299, miR381, |
| | miR-1246, miR-410, miR-196a, miR-429, miR-141, miR-376a, miR- |
| | 370, miR-200b, miR-125a-5p, miR-205, miR-200a, miR-224, miR- |
| | 494, miR-216a, miR-654-5p, miR-217, miR-99b, miR-885-3p, miR- |
| | 1228, miR-483-5p, miR-200c, miR-3065-5p, miR-203, miR-1308, |
| | let-7a, miR-17-92, miR-34a, miR-223, miR-150, miR-15b, miR- |
| | 199a-5p, miR-33a, miR-423-5p, miR-424, let-7d, miR-103, miR-23b, |
| | miR-30d, miR-425, miR-23a, miR-26a, miR-339-3p, miR-127-3p, |
| | miR-148b, miR-376a, miR-376c, miR-409-3p, miR-652, miR- 801 |
| | (miR-92a, miR-548d-5p, miR-760, miR-1234, miR-18b, miR-605, |
| | miR-193b, miR-29) |
| Leukemia | miR-98, miR-155, miR-21, let-7, miR-126, miR-196b, miR-128, |
| | miR-195, miR-29a, miR-222, miR-20a, miR-150, miR-451, miR- |
| | 135a, miR-486-5p, miR-92, miR-148a, miR-181a, miR-20a, miR- |
| | 221, miR-625, miR-99b |
| | (miR-92a, miR-15, miR-16, miR-15a, miR-16-1, miR-29) |
| Multiple myeloma | miR-15a, miR-16, miR-193b-365, miR-720, miR-1308, miR-1246, |
| | miR-1, miR-133a, miR-221, miR-99b, Let-7e, miR-125a-5p, miR- |
| | 21, miR-181a/b, miR-106b-25, miR-32, miR-19a/b, miR-17-92, miR- |
| | 17, miR-20, miR-92, miR-20a, miR-148a, miR-153, miR-490, miR- |
| | 455, miR-642, miR-500, miR-296, miR-548d, miR-373, miR-554, |
| | miR-888, miR-203, miR-342, miR-631, miR-200a, miR-34c, miR- |
| | 361, miR-9*, miR-200b, miR-9, miR-151, miR-218, miR-28-3p, |
| | miR-200c, miR-378, miR-548d-5p, miR-621, miR-140-5p, miR-634, |
| | miR-616, miR-130a, miR-593, miR-708, miR-200a*, miR-340, miR- |
| | 760, miR-188-5p, miR-760, miR-885-3p, miR-590-3p, miR-885-5p, |
| | miR-7, miR-338, miR-222, miR-99a, miR-891a, miR-452, miR-98, |
| | miR-629, miR-515-3p, miR-192, miR-454, miR-151-3p, miR-141, |
| | miR-128b, miR-1227, miR-128a, miR-205, miR-27b, miR-608, miR- |
| | 432, miR-220, miR-135a, miR-34a, miR-28, miR-412, miR-877, |
| | miR-628-5p, miR-532-3p, miR-625, miR-34b, miR-31, miR-106b, |
| | miR-146a, miR-210, miR-499-5p, miR-140, miR-188, miR-610, |
| | miR-27a, miR-142-5p, miR-603, miR-660, miR-649, miR-140-3p, |
| | miR-300, miR-335, miR-206, miR-20b, miR-130b, miR-183, miR- |
| | 652, miR-133b, miR-191, miR-212, miR-194, miR-100m miR- |
| | 1234m miR-182m miR-888, miR-30e-5p, miR-574, miR-135b, miR- |
| | 125b, miR-502m miR-320, miR548-421, miR-129-3p, miR-190b, |
| | miR-18a, miR-549, 338-5p, miR-756-3p, miR-133a, miR-521, miR- |
| | 486-3p, miR-553, miR-452*, miR-628-3p, miR-620, miR-566, miR- |
| | 892a, miR- miR-339-5p, miR-628, miR-520d-5p, miR-297, miR-213, |
| | miR-519e*, miR-422a, miR-198, miR-122a, miR-1236, miR-548c- |
| | 5p, miR-191*, miR-583, miR-376c, miR-34c-3p, miR-453, miR-509, |
| | miR-124a, miR-505, miR-208, miR-659, miR-146b, miR-518c, miR- |
| | 665, miR-324-5p, miR-152, miR-548d, miR-455-3p |
| | (miR-15a, miR-373*, miR-378*, miR-143, miR-337, miR-223, miR- |
| | 369-3p, miR-520g, miR-485-5p, miR-524, miR-520h, miR-516-3p, |
| | miR-519d, miR-371-3p, miR-455, miR-520b, miR-518d, miR-624, |
| | miR-296, miR-16) |
| monoclonal | miR-21, miR-210, miR-9*, miR-200b, miR-222, miR-376 |
| gammopathy of | (miR-339, miR-328) |
| undetermined | |
| significance | |
| Myelodisplastic | (Let-7a, miR-16) |
| syndrome | |
| Lymphoma | miR-155, miR-210, miR-21, miR-17-92, miR-18a, miR-181a, miR- |
| | 222, miR-20a/b, miR-194, miR-29, miR-150, miR-155, miR-223, |
| | miR-221, let-7f, miR-146a, miR-15, miR-16-1, miR-34b/c, miR-17-5p |
| | (miR-20b, miR-184, miR-200a/b/c, miR-205, miR-34a, miR-29a, |
| | miR-29b-1, miR-139, miR-345, miR-125a, miR-126, miR-26a/b, |
| | miR-92a, miR-20a, miR-16, miR-101, miR-29c miR-138, miR-181b) |
| Lung cancer | let-7c, miR-100, miR-10a, miR-10b, miR-122a, miR-125b, miR-129, |
| | miR-148a, miR-150, miR-17-5p, miR-183, miR-18a*, miR-18b, |
| | miR-190, miR-192, miR-193a, miR-196b, miR-197, miR-19a, miR- |
| | 19b, miR-200c, miR-203, miR-206, miR-20b, miR-210, miR-214, |
| | miR-218, miR-296, miR-30a-3p, miR-31, miR-346, miR-34c, miR- |
| | 375, miR-383, miR-422a, miR-429, miR-448, miR-449, miR-452, |
| | miR-483, miR-486, miR-489, miR-497, miR-500, miR-501, miR- |
| | 507, miR-511, miR-514, miR-516-3p, miR-520d, miR-527, miR-7, |
| | miR-92, miR-93, miR-99a, miR-25, miR-223, miR-21, miR-155, |
| | miR-556, miR-550, miR-939, miR-616*, miR-146b-3p and miR-30c-1*, |
| | miR-142-5p, miR-328, miR-127, miR-151, miR-451, miR-126, |
| | miR-425-5p, miR-222, miR-769-5p, miR-642, miR-202, miR-34a |
| | (let-7a, let-7d, let-7e, let-7g, let-7i, miR-1, miR-103, miR-106a, miR- |
| | 125a, miR-130a, miR-130b, miR-133a, miR-145, miR-148b, miR- |
| | 15a, miR-15b, miR-17-3p, miR-181d, miR-18a, miR-196a, miR-198, |
| | miR-199a, miR-199a*, miR-212, miR-22, miR-221, miR-23a, miR- |
| | 23b, miR-26a, miR-27a, miR-27b, miR-29b, miR-30b, miR-30d, |
| | miR-30e-3p, miR-320, miR-323, miR-326, miR-331, miR-335, miR- |
| | 339, miR-374, miR-377, miR-379, miR-410, miR-423, miR-433, |
| | miR-485-3p, miR-485-5p, miR-487b, miR-490, miR-491, miR-493, |
| | miR-493-3p, miR-494, miR-496, miR-502, miR-505, miR-519d, |
| | miR-539, miR-542-3p, miR-98) |
| Colorectal cancer | miR-29a, miR-17-3p, miR-92, miR-21, miR-31, miR-155, miR-92a, |
| | miR-141, mir-202, mir-497, mir-3065, mir-450a-2, mir-3154, mir- |
| | 585, mir-3175, mir-1224, mir-3117, mir-1286 |
| | (miR-34) |
| Prostate cancer | miR-141, miR-375, miR-16, miR-92a, miR-103, miR-107, miR-197, |
| | miR-485-3p, miR-486-5p, miR-26a, miR-92b, miR-574-3p, miR- |
| | 636, miR-640, miR-766, miR-885-5p, miR-141, miR-195, miR-375, |
| | miR-298, miR-346, miR-1-1, miR-1181, miR-1291, miR-133a-l, |
| | miR-133b, miR-1469, miR-148*, miR-153, miR-182, miR-182*, |
| | miR-183, miR- 183*, miR-185, miR-191, miR-192, miR-1973, miR- |
| | 200b, miR-205, miR-210, miR-33b*, miR-3607-5p, miR-3621, miR- |
| | 378a, miR-429, miR-494, miR-582, miR-602, miR-665, miR-96, |
| | miR-99b*, miR-100, miR-125b, miR-143, miR-200a, miR-200c, |
| | miR-222, miR-296, and miR-425-5p |
| Ovarian cancer | miR-21, miR-92, miR-93, miR-126, miR-29a, miR-141, miR- |
| | 200a/b/c, miR-203, miR-205, miR-214, miR-221, miR-222, miR- |
| | 146a, miR-150, miR- 193a-5p, miR-31, miR-370, let-7d, miR-508- |
| | 5p, miR-152, miR-509-3-5p, miR-508-3p, miR-708, miR-431, miR- |
| | 185, miR-124, miR-886-3p, hsa-miR-449, hsa-miR-135a, hsa-miR- |
| | 429, miR-205, miR-20b, hsa-miR-142-5p, miR-29c, miR-182 |
| | (miR-155, miR-127, miR-99b) |
| Cervical cancer | miR-21, miR-9, miR-200a, miR-497 |
| | (miR-143, miR-203, miR-218) |
| Esophageal carcinoma | miR-21, hsa-miR-200a, hsa-miR-345, hsa-miR-373*, hsa-miR-630, |
| | hsa-miR-663, hsa-miR-765, hsa-miR-625, hsa-miR-93, hsa-miR- |
| | 106b, hsa-miR-155, hsa-miR-130b, hsa-miR-30a, hsa-miR-301a, |
| | hsa-miR-15b |
| | (miR-375) |
| Gastric cancer | miR-17-5p, miR-21, miR-106a, miR-106b, miR-187, miR-371-5p, |
| | miR-378 |
| | (let-7a, miR-31, miR-192, miR-215, miR-200/141) |
| Pancreatic cancer, | miR-210, miR-21, miR-155, miR-196a, miR-1290, miR-20a, miR- |
| ductal adenocarcinoma | 24, miR-25, miR-99a, miR-185, miR-191, miR-18a, miR-642b-3p, |
| | miR-885-5p, miR-22-3p, miR-675, miR-212, miR-148a*, miR-148, |
| | miR-187, let-7g*, miR-205, miR-944, miR-431, miR-194*, miR- |
| | 769-5p, miR-450b-5p, miR-222, miR-222*, miR-146, miR-23a*, |
| | miR-143*, miR-216a, miR-891a, miR-409-5p, miR-449b, miR-330- |
| | 5p, miR-29a*, miR-625 |
| Hepatocellular | miR-500, miR-15b, miR-21, miR-130b, miR-183, miR-122, miR- |
| carcinoma | 34a, miR-16, miR-221, miR-222 |
| Melanoma | miR-150, miR-15b, miR-199a-5p, miR-33a, miR-423-5p, miR-424, |
| | miR- let-7d, miR-103, miR-23b, miR-30d, miR-425, miR-222, miR- |
| | 23a, miR-26a, miR-339-3p |
| Squamous cell | miR-184a |
| carcinoma | |
| Bladder cancer | miR-126, miR-182 (urine), miR-16, miR-320 |
| | (miR-143, miR-145, miR-200/141) |
| Renal cancer | miR-1233, miR-199b-5p, miR-130b |
| | (miR-10b, miR-139-5p) |
| Oral cancer | miR-31, miR-24, miR-184; miR-34c; miR-137; miR-372; miR-124a; |
| | miR-21; miR-124b; miR-31; miR-128a; miR-34b; miR-154; miR- |
| | 197; miR-132; miR-147; miR-325; miR-181c; miR-198; miR-155; |
| | miR-30a-3p; miR-338; miR-17-5p; miR-104; miR-134; miR-213 |
| | (miR-200a, miR-125a, miR-133a; miR-99a; miR-194; miR-133; |
| | miR-219; miR-100; miR-125; miR-26b; miR-138; miR-149; miR- |
| | 195; miR-107; and miR-139 (saliva)) |
| Head and neck cancer | miR-455-3p, miR-455-5p, miR-130b, miR-130b*, miR-801, miR- |
| | 196a, miR-21, miR-31 |
| Endometrial cancer | miR-503, miR-424, miR-29b, miR-146a, miR-31 |
| Testicular cancer | miR-372, miR-373 |
| Glioblastoma | miR-21, miR-221, miR-222 |
| Thyroid cancer | miR-187, miR-221, miR-222, miR-146b, miR-155, miR-224, miR- |
| | 197, miR-192, miR-328, miR-346, miR-512-3p, miR-886-5p, miR- |
| | 450a, miR-301 b, miR-429, miR-542-3p, miR-130a, miR-146b-5p, |
| | miR-199a-5p, miR-193a-3p, miR-152, miR-199a-3p/miR-199b-3p, |
| | miR-424, miR-22, miR-146a, miR-339-3p, miR-365, let-7i*, miR- |
| | 363*, miR-148a, miR-299-3p, let-7a*, miR-200b, miR-200c, miR- |
| | 375, miR-451 , miR-144, let-7i, miR-1826, miR-1201, miR-140-5p, |
| | miR-126, miR-126*, let-7f-2*, miR-148b, miR-21*, miR-342- 3p, |
| | miR-27a, miR-145*, miR-513b, miR-101, miR-26a, miR-24, miR- |
| | 30a*, miR-377, miR-518e7, miR-519a7, miR-519b-5p, miR-519c- |
| | 5p, miR-5227, miR-523*, miR-222*, miR-452, miR-665, miR-584, |
| | miR-492, miR-744, miR-662, miR-219-2-3p, miR-631 and miR-637, |
| | miRPIus-E1078, miR-19a, miR-501-3p, miR-17, miR-335, miR- |
| | 106b, miR-15a, miR-16, miR-374a, miR-542-5p, miR-503, miR- |
| | 320a, miR-326, miR-330-3p, |
| | miR-1, miR-7b, miR-26b, miR-106a, miR-139, miR-141, miR-143, |
| | miR-149, miR-182, miR-190b, miR-193a, miR-193b, miR-211, miR- |
| | 214, miR-218, miR-302c*, miR-320, miR-324, miR-338, miR-342, |
| | miR-367, miR-378, miR-409, miR-432, miR-483, miR-486, miR- |
| | 497, miR-518f, miR-574, miR-616, miR-628, miR-663b, miR-888, |
| | miR-1247, miR-1248, miR-1262, and miR-1305 |
| | miR-21, miR-25, miR-32, miR-99b*, miR-125a, miR-125b, miR- |
| | 138, miR-140, miR-181a, miR-213, miR-221, miR-222, and miR-345 |
| Ischemic heart disease/ | miR-1, miR-30c, miR-133, miR-145, miR-208a/b, miR-499, miR- |
| Myocardial infarction | 663b, miR-1291 |
| | (miR-126, miR-197, miR-223) |
| Heart failure | miR-29b, miR-122, miR-142-3p, miR-423-5p, miR-152, miR-155, |
| | miR-497 |
| | (miR-107, miR-125b, miR-126, miR-139, miR-142-5p, miR-497) |
| Stroke | miR-124, miR-145 |
| | (miR-210) |
| Coronary artery disease | miR-21, miR-27b, miR-130a, miR-134, miR-135a, miR-198, miR- |
| | 210, miR-370 |
| | (miR-17, miR-92a, miR-126, miR-145m miR-155m miR-181a, miR- |
| | 221, miR-222) |
| Diabetes | miR-9, miR-28-3p, miR-29a, miR-30d, miR-34a, miR-124a, miR- |
| | 146a, miR-375, miR-503, 144 |
| | (miR-15a, miR-20b, miR-21, miR-24, miR-126, miR-191, miR-197, |
| | 223, miR-320, miR-486) |
| Hypertension | Hcmv-miR-UL112, Let-7e |
| | (miR-296-5p) |
| Chronic HCV infection | miR-155, miR-122, miR-125b, miR-146a, miR-21 |
| Liver injury | miR-122, miR-192 |
| Sepsis | miR-146a, miR223 |
| Arthritis | miR-125a-5p, miR-24, miR-26a, miR-9, miR-25, miR-98, miR-146a, |
| | miR-124a, miR-346, miR-223, miR-155 |
| | (miR-132, miR-146) |
| Systemic lupus | (miR-200a/b/c, miR-205, miR-429, miR-192, miR-141, miR-429, |
| erythematosus | miR-192 (urine or serum)) |
| Chron disease | miR-199a-5p, miR-362-3p, miR-532-3p, miR-plus-E1271, miR-340* |
| | (miR-149*, miR-plus-F1065) |
| Ulcerative colitis | miR-28-5p, miR-151-5p, miR-199-5p, miR-340*, miR-plus-E1271, |
| | miR-103-2*, miR-362-3p, miR-532-3p |
| | (miR-505) |
| Asthma | miR-705, miR-575, let-7d, miR-173p, miR-423-5p, miR-611, miR- |
| | 674, let-7f-1, miR-23b, miR-223, miR-142-3p, let-7c, miR-25, miR- |
| | 15b, let-7g, and miR-542-5p, miR-370 |
| | (miR-325, miR-134, miR-198, miR-721, miR-515-3p, miR-680, |
| | miR-601, miR-206, miR-202, miR-671, miR-381, miR-630, miR- |
| | 759, miR-564, miR-709, miR-513, miR-298) |
| Chronic pulmonary | miR-148a, miR-148b, miR-152 |
| disease | |
| Idiopathic pulmonary | miR-199a-5p |
| fibrosis | |
| Alzheimer's disease | (miR-137, miR-181c, miR-9, miR-29a/b) |
| Duchenne muscular | miR-1, miR-133a, miR-206 |
| dystrophy | |
| Multiple sclerosis | miR-633, miR-181c-5p (CSF), miR-17-5p, miR-193a, miR-326, |
| | miR-650, miR-155, miR-142-3p, miR-146a, miR-146b, miR-34a, |
| | miR-21, miR-23a, miR-199a, miR-27a, miR-142-5p, miR-193a, |
| | miR-15a, miR-200c, miR-130a, miR-223, miR-22, miR-320, miR- |
| | 214, miR-629, miR-148a, miR-28, miR-195, miR-135a, miR-204, |
| | miR-660, miR-152, miR-30a-5p, miR-30a-3p, miR-365, miR-532, |
| | let-7c, miR-20b, miR-30d, miR-9, hsa-mir-18b, hsa-mir-493, hsa- |
| | mir-599, hsa-mir-96, hsa-mir-193, hsa-mir-328, hsa-mir-409-5p, |
| | hsa-mir-449b, hsa-mir-485-3p, hsa-mir-554 |
| | (miR-922 (CSF), miR-497, miR-1 and miR-126, miR-656, miR-184, |
| | miR-139, miR-23b, miR-487b, miR-181c, miR-340, miR-219, miR- |
| | 338, miR-642, miR-181b, miR-18a, miR-190, miR-213, miR-330, |
| | miR-181d, miR-151, miR-140) |
| Preeclampsia | miR-210 |
| | (miR-152) |
| Gestational diabetes | (miR-29a, miR-132) |
| Platelet activity | miR-126, miR-197, miR-223, miR-24, miR-21 |
| Pregnancy/placenta- | miR-526a, miR-527, miR-520d-5p, miR-141, miR-149, miR-299-5p, |
| derived | miR-517a |
| Drug treatment for | miR-130a, miR-146b, miR-143, miR-145, miR-99b, miR-125a, miR- |
| immunomodulation | 204, miR-424, miR-503 |
| Aging | (miR-151a-3p, miR-181a-5p, miR-1248) |

*miRNA markers in parentheses are downregulated

The subject method also finds use in validation assays. For example, validation assays may be used to validate or confirm that a potential disease biomarker is a reliable indicator of the presence or absence of a disease across a variety of individuals. The short assay times for the subject method may facilitate an increase in the throughput for screening a plurality of samples in a minimum amount of time.

In some instances, the subject method can be used without requiring a laboratory setting for implementation. In comparison to the equivalent analytic research laboratory equipment, the subject method provides comparable analytic sensitivity in a portable, hand-held system. In some cases, the mass and operating cost are less than the typical stationary laboratory equipment. In addition, the subject method can be utilized in a home setting for over-the-counter home testing by a person without medical training to detect one or more analytes in samples. The subject method may also be utilized in a clinical setting, e.g., at the bedside, for rapid diagnosis or in a setting where stationary research laboratory equipment is not provided due to cost or other reasons.

As noted above, a subject signal-amplifying nanosensor can be used to detect nucleic acids in a sample. A subject signal-amplifying nanosensor may be employed in a variety of drug discovery and research applications in addition to the diagnostic applications described above. For example, a subject signal-amplifying nanosensor may be employed in a variety of applications that include, but are not limited to, diagnosis or monitoring of a disease or condition (where the presence of an nucleic acid provides a biomarker for the disease or condition), discovery of drug targets (where, e.g., an nucleic acid is differentially expressed in a disease or condition and may be targeted for drug therapy), drug screening (where the effects of a drug are monitored by assessing the level of an nucleic acid), determining drug susceptibility (where drug susceptibility is associated with a particular profile of nucleic acids) and basic research (where is it desirable to identify the presence a nucleic acid in a sample, or, in certain embodiments, the relative levels of a particular nucleic acids in two or more samples).

In certain embodiments, relative levels of nucleic acids in two or more different nucleic acid samples may be obtained using the above methods, and compared. In these embodiments, the results obtained from the above-described methods are usually normalized to the total amount of nucleic acids in the sample (e.g., constitutive RNAs), and compared. This may be done by comparing ratios, or by any other means. In particular embodiments, the nucleic acid profiles of two or more different samples may be compared to identify nucleic acids that are associated with a particular disease or condition.

In some examples, the different samples may consist of an “experimental” sample, i.e., a sample of interest, and a “control” sample to which the experimental sample may be compared. In many embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment of the invention, the experimental material is cells susceptible to infection by a pathogen such as a virus, e.g., human immunodeficiency virus (HIV), etc., and the control material is cells resistant to infection by the pathogen. In another embodiment of the invention, the sample pair is represented by undifferentiated cells, e.g., stem cells, and differentiated cells.

As described above, aspects of the subject method include providing or receiving a report that indicates the measured amount of the analyte, e.g., a biomarker, in the sample. In some cases, where the sample is a diagnostic sample, the report may also include a range of measured values for the biomarker in an individual free of or at low risk of having the disease or condition, wherein the measured amount of the biomarker in the diagnostic sample obtained from the subject relative to the range of measured values obtained from healthy individuals is diagnostic of a disease or condition. In such instances, if the measured value of the biomarker in a sample provided by a subject falls outside the range of expected values for the biomarker in a healthy individual, the subject may have a higher chance of being predisposed to or having the disease or condition. In some cases, the measured amount of the biomarker and the range of values obtained from healthy individuals are normalized to a predetermined standard to allow comparison.

In certain aspects, the report may indicate to the subject the presence or absence of a biomarker, the concentration of a biomarker, the presence or absence of disease or a condition, the probability or likelihood that the subject has a disease or a condition, the likelihood of developing a disease or a condition, the change in likelihood of developing a disease or a condition, the progression of a disease or a condition, etc. The disease or condition reported may include, but are not limited to: cancer; inflammatory disease, such as arthritis; metabolic disease, such as diabetes; ischemic disease, such as stroke or heart attack; neurodegenerative disease, such as Alzheimer's Disease or Parkinson's Disease; organ failure, such as kidney or liver failure; drug overdose; stress; fatigue; muscle damage; pregnancy-related conditions, such as non-invasive prenatal testing, etc. In certain embodiments, the report contains instructions urging or recommending the patient to take action, such as seek medical help, take medication, stop an activity, start an activity, etc. The report may include an alert. One example of an alert may be if an error is detected on the device, or if an analyte concentration exceeds a predetermined threshold. The content of the report may be represented in any suitable form, including text, graphs, graphics, animation, color, sound, voice, and vibration.

In certain embodiment, the report provides an action advice to the user of the subject device, e.g., a mobile phone. The advices will be given according to the test data by the devices (e.g. detectors plus mobile phone) together with one or several data sets, including but not limited to, the date preloaded on the mobile devices, data on a storage device that can be accessed, where the storage device can be locally available or remotely accessible.

The advices include, but not limited to, one of the following: (i) normal (have a good day), (ii) should be monitored frequently; (iii) the following parameters should be checked closely (and list the parameters), (iv) should check every day, because subject's specific parameters on the boarder lines, (v) should visit doctor within certain days, because specific parameters are mild above to the threshold; (vi) should see doctor immediately, and (vii) should go to an emergency room immediately.

In some embodiments, when the device concludes that a subject needs to see a physician or go an emergency room, the device automatically sends such request to a physician and an emergency room.

In some embodiments, when the automatically sent request by the devices are not responded by a physician or an emergency room, the device will repeatedly send the request in certain time interval.

In certain embodiments, the report may provide a warning for any conflicts that may arise between an advice based on information derived from a sample provided by a subject and any contraindications based on a health history or profile of the subject.

In certain embodiments, the subject method includes diagnosing a subject based on information including the measured amount of the biomarker in the sample provided by the subject. In addition to data related to the measured biomarker in the sample (e.g., type of biomarker, amount of biomarker in the sample), the information used to to diagnose a subject may also include other data related to the subject, including but not limited to the age, sex, height, weight, or individual and/or family medical history, etc. of the subject.

In some embodiments, the diagnosing step includes sending data comprising the measured amount of the biomarker to a remote location and receiving a diagnosis from the remote location. Diagnosing the subject based on information including the biomarker detected by the signal-amplifying nanosensor may be achieved by any suitable means. In certain embodiments, the diagnosing is done by a health care professional who may be with the subject or may be at the remote location. In other embodiments, a health care professional has access to the data transmitted by the device at a third location that is different from the remote location or the location of the subject. A health care professional may include a person or entity that is associated with the health care system. A health care professional may be a medical health care provider. A health care professional may be a doctor. A health care professional may be an individual or an institution that provides preventive, curative, promotional or rehabilitative health care services in a systematic way to individuals, families and/or communities. Examples of health care professionals may include physicians (including general practitioners and specialists), dentists, physician assistants, nurses, midwives, pharmaconomists/pharmacists, dietitians, therapists, psychologists, chiropractors, clinical officers, physical therapists, phlebotomists, occupational therapists, optometrists, emergency medical technicians, paramedics, medical laboratory technicians, medical prosthetic technicians, radiographers, social workers, and a wide variety of other human resources trained to provide some type of health care service. A health care professional may or may not be certified to write prescriptions. A health care professional may work in or be affiliated with hospitals, health care centers and other service delivery points, or also in academic training, research and administration. Some health care professionals may provide care and treatment services for patients in private homes. Community health workers may work outside of formal health care institutions. Managers of health care services, medical records and health information technicians and other support workers may also be health care professionals or affiliated with a health care provider.

In some embodiments, the health care professional may already be familiar with the subject or have communicated with the subject. The subject may be a patient of the health care professional. In some instances, the health care professional may have prescribed the subject to undergo a clinical test. In one example, the health care professional may be the subject's primary care physician. The health care professional may be any type of physician for the subject (including general practitioners, and specialists).

Thus, a health care professional may analyze or review the report generated by the device that acquired the light signal from a signal-amplifying nanosensor device, or the data transmitted from the device and/or the results of an analysis performed at a remote location. In certain embodiments, the health care professional may send to the subject instructions or recommendations based on the data transmitted by the device and/or analyzed at the remote location.

### Environmental Testing

As summarized above, the present method may find use in analyzing an environmental sample, e.g., a sample from water, soil, industrial waste, etc., for the presence of environmental markers. An environmental marker may be any suitable marker, such as those shown in Table 8, below, that can be captured by a capturing agent that specifically binds the environmental marker in a signal-amplifying nanosensor configured with the capturing agent. The environmental sample may be obtained from any suitable source, such as a river, ocean, lake, rain, snow, sewage, sewage processing runoff, agricultural runoff, industrial runoff, tap water or drinking water, etc. In some embodiments, the presence or absence, or the quantitative level of the environmental marker in the sample may be indicative of the state of the environment from which the sample was obtained. In some cases, the environmental marker may be a substance that is toxic or harmful to an organism, e.g., human, companion animal, plant, etc., that is exposed to the environment. In some cases, the environmental marker may be an allergen that may cause allergic reactions in some individuals who are exposed to the environment. In some instances, the presence or absence, or the quantitative level of the environmental marker in the sample may be correlated with a general health of the environment. In such cases, the general health of the environment may be measured over a period of time, such as week, months, years, or decades.

In some embodiments, the present method further includes receiving or providing a report that indicates the safety or harmfulness for a subject to be exposed to the environment from which the sample was obtained based on information including the measured amount of the environmental marker. The information used to assess the safety risk or health of the environment may include data other than the type and measured amount of the environmental marker. These other data may include the location, altitude, temperature, time of day/month/year, pressure, humidity, wind direction and speed, weather, etc. The data may represent an average value or trend over a certain period (minutes, hours, days, weeks, months, years, etc.), or an instantaneous value over a shorter period (milliseconds, seconds, minutes, etc.).

The report may be generated by the device configured to read the signal-amplifying nanosensor, or may be generated at a remote location upon sending the data including the measured amount of the environmental marker. In some cases, an expert may be at the remote location or have access to the data sent to the remote location, and may analyze or review the data to generate the report. The expert may be a scientist or administrator at a governmental agency, such as the US Centers for Disease Control (CDC) or the US Environmental Protection Agency (EPA), a research institution, such as a university, or a private company. In certain embodiments, the expert may send to the user instructions or recommendations based on the data transmitted by the device and/or analyzed at the remote location.

TABLE 8
Environmental Markers

| CLASS/SOURCE | MARKER |
|---|---|
| Synthetic hormone | 17beta-estradiol (E2), estrone (El), estrogen (ES: El + E2 + estriol |
| analogues | (E3)), 1 7alfa-ethynylestradiol (EE2), 4-nonylphenpol, testosterone |
| Halogenated | p,p′-DDE, p,p′-DDD, p,p′-DDT, o,p′-DDE, o,p′-DDE, o,p′-DDT, |
| hydrocarbons | o,p′-DDD, chlordane, nonachlor, oxychlordane, heptachlor, heptachlor |
| | epoxide, pentachloroanisole, hexachlorobenzene, heptachlorbenzene, |
| | o,p′-methoxychlor, p,p′-methoxychlor, Hexachlorocyclopentadiene |
| Pesticides | manganese ethylene-bis-dithiocarbamate, diazinon, chlorphyrifos, |
| | carbofuran, carbaryl, malathion, dieldrin, fipronil, desulfinylfipronil, |
| | fipronil sulfide, fipronil sulfone, aldicarb, aldicarb sulfone, aldicarb |
| | sulfoxide, carbaryl, 3-hydroxycarbofuran, methiocarb, methomyl,, |
| | oxamyl, propoxur, alpha-HCH, gamma-HCH, beta-HCH, delta-HCH, |
| | azinphos-methyl, chlorpyrifos, disulfoton, parathion, fonofos, |
| | ethoprop, parathion-methyl, phorate, terbufos, cis-permethrin, trans- |
| | permethrin, propargite, aldrin, chloroneb, endosulfan I, endrin, isodrin, |
| | mirex, toxaphene, lindane, O-ethyl O-4-nitrophenyl phenylphosphono- |
| | thioate (EPN), fenitrothion, pirimiphos-methyl, deltamethrin |
| Herbicide | acetochlor, alachlor, metolachlor, atrazine, deethylatrazine, cyanazine, |
| | terbuthylazine, terbutryn, metribuzin, bentazon, EPTC, triflualin, |
| | molinate norflurazon, simazine, prometon, promteryn, tebuthiuron, |
| | 2,4-D, diuron, dacthal, bromacil, deisopropyl atrazine, |
| | hydroxyatrazine, deethylhydroxyatrazine, deisopropylhydroxyatrazine, |
| | acetochlor ESA, acetochlor OA, alachlor ESA, alachlor OA, |
| | metolachlor ESA, metolachlor OA, 2,6-diethylaniline, napropamide, |
| | pronamide, propachlor, propanilm butylate, pebulate, propham, |
| | thiobencarb, triallate, dacthal, dacthal monoacid, 2,4-DB, |
| | dischlorprop, MCPA, MCPB, 2,4,5-T, 2,4,5-TP, benfluralin, |
| | ethalfluralin, oryzalin, pendimethalin, trifluralin, bentazon, |
| | norflurazon, acifluorfen, chloramben methyl ester, clopyralid, |
| | dicamba, picloram, dinoseb, DNOC, chlorothalonil, dichlobenil, 2,6- |
| | dichlorobenzamide (BAM), triclopyr, bromoxynil, bromacil, terbacil, |
| | fenuron, fluometuron, linuron, neburon, dalapon, diquat, endothall, |
| | Glyphosate, N-dealkylated triazines, mecoprop |
| Industrial material/waste | chromated copper arsenate, Carbon tetrachloride, Chlorobenzene, p- |
| | Dichlorobenzene, 1,2-Dichloroethanem, 1,1-Dichloroethylene, cis- |
| | 1,2-Dichloroethylene, trans-1,2-Dichloroethylene, Dichloromethane, |
| | Di(2-ethylhexyl) adipate, Di(2-ethylhexyl) phthalate, Dibutyl |
| | phthalate (DBP), diethyl phthalate (DEP), dicyclohexyl phthalate |
| | (DCHP), Dioxin (2,3,7,8-TCDD), Epichlorohydrin, Ethylene |
| | dibromide, Polychlorinated biphenyls, Pentachlorophenol, styrene, |
| | Tetrachloroethylene, Toluene diisocyanate (TDI), 1,2,4- |
| | Trichlorobenzene, 1,1,1-Trichloroethane, 1,1,2-Trichloroethane, |
| | Trichloroethylene, perchloroethylene, Vinyl chloride, Xylenes, |
| | alkylphenol (AP), AP + APE, bisphenol A (BPA), benzene, Xylene, |
| | Toluene, Styrene, Toluidine, 2-(p-Tolyl)ethylamine, Ethylbenzene, 2- |
| | Methyl-naphthalene, and Propyl-benzene, PAH (polynuclear aromatic |
| | hydrocarbons) |
| Drinking water | Bromate, Chlorite, Haloacetic acids, Total Trihalomethanes, |
| | Chloramines, Chlorine, Chlorine dioxide, Benzo(a)pyrene, 4-tert- |
| | octylphenol |
| Household waste/ | Acrylamide, linear alkylbenzene sulfonates (LAS), alkyl ethoxylates |
| Sewage runoff | (AE), alkylphenol ethoxylates (APE), triclosan |
| Poison/toxins | N-methylamino-L-alanine (BMAA), Clostridium botulinum |
| | neurotoxins, BoNT A, B, D, E, Ricin A, B, tetanus toxin, diphtheria |
| | toxin, pertussis toxin |
| Heavy metal | mercury/methylmercury, lead/tetraethyl lead, zinc, copper, nickel, |
| | cadmium, chromium(VI)/chromate, aluminum, iron, arsenic, cobalt, |
| | selenium, silver, antimony, thallium, polonium, radium, tin, |
| | metallothionein (in carp liver tissue) |
| Other metals/inorganic | Lithium, beryllium, manganese, barium, cyanide, fluoride |
| chemicals | |
| Pathogens/microbes | Anthrax (LF), Giardia lamblia, Legionella, Total Coliforms (including |
| (antigen in pretheses) | fecal coliform and E. Coli), Viruses (enteric) stapylococci (e.g., |
| | Staphylococcus epidermidis and Staphylococcus aureus (enterotoxin |
| | A, B, C, G, I, cells, TSST-1), Enterrococcus faecalis, Pseudomonas |
| | aeruginosa, Escherichia coli (Shiga-like toxin, F4, F5, H, K, O, |
| | bacteriophage K1, K5, K13), other gram-positive bacteria, and gram- |
| | negative bacilli. Clostridium difficile (Toxin A, B) |
| | Bacteroidetes, Cryptosporidium parvum (GP900, p68 or cryptopain, |
| | oocyst), Candida albicans |
| | Bacillus anthracis, Bacillus stearothermophilus |
| | Norovirus, Listeria monocytogenes (internalin), Leptospira |
| | interrogans, Leptospira biflexa, Clostridium perfringens (Epsilon |
| | toxin), Salmonella typhimurium, Yersinia pestis (F1, V antigens), |
| | Aspergillus flavus (aflatoxin), Aspergillus parasiticus (aflatoxin), |
| | avian influenza virus, Ebola virus (GP), Histoplasma capsulatum, |
| | Blastomyces dermatitidis (A antigen) |
| | Gram-positive bacteria (teichoic acid), Gram-ngative bacteria (such as |
| | Pseudomonas aeruginosa, Klebsiella pneumoniae, Salmonella |
| | enteriditis, Enterobacter aerogenes, Enterobacter hermanii, Yersinia |
| | enterocolitica and Shigella sonnei)(LPS), Polio virus, Influenza type A |
| | virus |
| | Disease specific prion (PrP-d) |
| Allergens | mite (Acas13, Blot1, Blot3, Blot4, Blot5, Blot6, Blot10, Blot11, |
| | Blot12, Blot13, Blot19); American house dust mite (Derf1, Derf2, |
| | Derf3, Derf7, Derf10, Derf11, Derf14, Derf15, Derf16, Derf17, |
| | Derf18w); house dust mite (Derm1); European house dust mite |
| | (Derp1, Derp2, Derp3, Derp4, Derp5, Derp6, Derp7, Derp8, Derp9, |
| | Derp10, Derp11, Derp14, Derp20, Derp21); mite (Eurm2; Eurm14); |
| | storage mite (Glyd2, Lepd2, Lepd5, Lepd7, Lepd10, Lepd13, Tyrp2, |
| | Tyrp13); Dermatophagoides farinae (Derf1.0101, Derf1.0102, |
| | Derf1.0103, Derf1.0104, Derf1.0105, Derf2.0101, Derf2.0102, |
| | Derf2.0103, Derf2.0104, Derf2.0105, Derf2.0106, Derf2.0107, |
| | Derf2.0108, Derf2.0109, Derf2.0110, Derf2.0111, Derf2.0112, |
| | Derf2.0113, Derf2.0114, Derf2.0115, Derf2.0116, Derf2.0117); |
| | Dermatophagoides pteronyssinus (Derp1.0101, Derp1.0102, |
| | Derp1.0103, Derp1.0104, Derp1.0105, Derp1.0106, Derp1.0107, |
| | Derp1.0108, Derp1.0109, Derp1.0110, Derp1.0111, Derp1.0112, |
| | Derp1.0113, Derp1.0114, Derp1.0115, Derp1.0116, Derp1.0117, |
| | Derp1.0118, Derp1.0119, Derp1.0120, Derp1.0121, Derp1.0122, |
| | Derp1.0123, Derp2.0101, Derp2.0102, Derp2.0103, Derp2.0104, |
| | Derp2.0105, Derp2.0106, Derp2.0107, Derp2.0108, Derp2.0109, |
| | Derp2.0110, Derp2.0111, Derp2.0112, Derp2.0113); Euroglyphus |
| | maynei (Eurm2.0101, Eurm2.0102); Glycyphagus domesticus |
| | (Glyd2.0101, Glyd2.0201); and Lepidoglyphus destructor |
| | (Lepd2.0101, Lepd2.0101, Lepd2.0101, Lepd2.0102, Lepd2.0201, |
| | Lepd2.0202) |
| | Pollen (Short Ragweed (Ambrosia artemisiifolia) allergen, Amb a 1, |
| | Amba2, Amba3, Amba5, Amba6, Amba7, Amba8, Amba9, Amba10; |
| | Betula verrucosa allergen, Bet v 1, Phleum pratense allergen, Phl p 5), |
| | giant ragweed (Ambt5); mugwort (Artv1, Artv2, Artv3, Artv4, Artv5, |
| | Artv6); sunflower (Hela1, Hela2, Hela3); Mercurialis annua (Mera1); |
| | lamb's-quarters, pigweed (Chea1); white goosefoot (Chea2, Chea3); |
| | Russian-thistle (Salk1); Rosy periwinkle (Catr1); English plantain |
| | (Plal1); Japanese hop (Humj1); Parietaria judaica (Parj1, Parj2, Parj3); |
| | Parietaria officinalis (Paro1); Ambrosia artemisiifolia (Amba8.0101, |
| | Amba8.0102, Amba9.0101, Amba9.0102); Plantago lanceolata |
| | (Plal1.0101, Plal1.0102, Plal1.0103); and Parietaria judaica |
| | (Parj1.0101, Parj1.0102, Parj1.0201, Par2.0101, Parj2.0102, |
| | Parj3.0101, Parj3.0102), Bermuda grass (Cynd1, Cynd7, Cynd12, |
| | Cynd15, Cynd22w, Cynd23, Cynd24); orchard grass (Dacg1, Dacg2, |
| | Dacg3, Dacg5); meadow fescue (Fesp4w); velvet grass (Holl1); rye |
| | grass (Lolp1, Lolp2, Lolp3, Lolp5, Lolp11); canary grass (Phaa1); |
| | Timothy (Phlp1, Phlp2, Phlp4, Phlp5, Phlp6, Phlp11, Phlp12, Phlp13); |
| | Kentucky blue grass (Poap1, Poap5); Johnson grass (Sorh1); Cynodon |
| | dactylon (Cynd1.0101, Cynd1.0102, Cynd1.0103, Cynd1.0104, |
| | Cynd1.0105, Cynd1.0106, Cynd1.0107, Cynd1.0201, Cynd1.0202, |
| | Cynd1.0203, Cynd1.0204); Holcus lanatus (Holl1.0101, Holl1.0102); |
| | Lolium perenne (Lolp1.0101, Lolp1.0102, Lolp1.0103, Lolp5.0101, |
| | Lolp5.0102); Phleum pretense (Phlp1.0101, Phlp1.0102, Phlp4.0101, |
| | Phlp4.0201, Phlp5.0101, Phlp5.0102, Phlp5.0103, Phlp5.0104, |
| | Phlp5.0105, Phlp5.0106, Phlp5.0107, Phlp5.0108, Phlp5.0201, |
| | Phlp5.0202); and Secale cereale (Secc20.0101, Secc20.0201), Alder |
| | (Alng1); Birch (Betv1, Betv2, Betv3, Betv4, Betv6, Betv7); hornbeam |
| | (Carb1); chestnut (Cass1, Cass5, Cass8); hazel (Cora1, Cora2, Cora8, |
| | Cora9, Cora10, Cora11); White oak (Quea1); Ash (Frae1); privet |
| | (Ligv1); olive (Olee1, Olee2, Olee3, Olee4, Olee5, Olee6, Olee7, |
| | Olee8, Olee9, Olee10); Lilac (Syrv1); Sugi (Cryj1, Cryj2); cypress |
| | (Cupa1); common cypress (Cups1, Cups3w); mountain cedar (Juna1, |
| | Juna2, Juna3); prickly juniper (Juno4); mountain cedar (Juns1); |
| | eastern red cedar (Junv1); London plane tree (Plaa1, Plaa2, Plaa3); |
| | date palm (Phod2); Betula verrucosa (Betv1.0101, Betv1.0102, |
| | Betv1.0103, Betv1.0201, Betv1.0301, Betv1.0401, Betv1.0402, |
| | Betv1.0501, Betv1.0601, Betv1.0602, Betv1.0701, Betv1.0801, |
| | Betv1.0901, Betv1.1001, Betv1.1101, Betv1.1201, Betv1.1301, |
| | Betv1.1401, Betv1.1402, Betv1.1501, Betv1.1502, Betv1.1601, |
| | Betv1.1701, Betv1.1801, Betv1.1901, Betv1.2001, Betv1.2101, |
| | Betv1.2201, Betv1.2301, Betv1.2401, Betv1.2501, Betv1.2601, |
| | Betv1.2701, Betv1.2801, Betv1.2901, Betv1.3001, Betv1.3101, |
| | Betv6.0101, Betv6.0102); Carpinus betulus (Carb1.0101, Carb1.0102, |
| | Carb1.0103, Carb1.0104, Carb1.0105, Carb1.0106, Carb1.0106, |
| | Carb1.0106, Carb1.0106, Carb1.0107, Carb1.0107, Carb1.0108, |
| | Carb1.0201, Carb1.0301, Carb1.0302); Corylus avellana (Cora1.0101, |
| | Cora1.0102, Cora1.0103, Cora1.0104, Cora1.0201, Cora1.0301, |
| | Cora1.0401, Cora1.0402, Cora1.0403, Cora1.0404); Ligustrum |
| | vulgare (Ligv1.0101, Ligv1.01.02); Olea europea (Olee1.0101, |
| | Olee1.0102, Olee1.0103, Olee1.0104, Olee1.0105, Olee1.0106, |
| | Olee1.0107); Syringa vulgaris (Syrv1.0101, Syrv1.0102, Syrv1.0103); |
| | Cryptomeria japonica (Cryj2.0101, Cryj2.0102); and Cupressus |
| | sempervirens (Cups1.0101, Cups1.0102, Cups1.0103, Cups1.0104, |
| | Cups1.0105) |
| | mold (Alternaria alternata allergen, Alt a 1, Alta3, Alta4, Alta5, |
| | Alta6, Alta7, Alta8, Alta10, Alta12, Alta13, Aspergillus fumigatus |
| | allergen, Asp f 1, Aspf2, Aspf3, Aspf4, Aspf5, Aspf6, Aspf7, Aspf8, |
| | Aspf9, Aspf10, Aspf11, Aspf12, Aspf13, Aspf15, Aspf16, Aspf17, |
| | Aspf18, Aspf22w, Aspf23, Aspf27, Aspf28, Aspf29); Aspergillus |
| | niger (Aspn14, Aspn18, Aspn25); Aspergillus oryzae (Aspo13, |
| | Aspo21); Penicillium brevicompactum (Penb13, Penb26); Penicillium |
| | chrysogenum (Pench13, Pench18, Pench20); Penicillium citrinum |
| | (Penc3, Penc13, Penc19, Penc22w, Penc24); Penicillium oxalicum |
| | (Peno18); Fusarium culmorum (Fusc1, Fusc2); Trichophyton rubrum |
| | (Trir2, Trir4); Trichophyton tonsurans (Trit1, Trit4); Candida albicans |
| | (Candal, Canda3); Candida boidinii (Candb2); Psilocybe cubensis |
| | (Psic1, Psic2); shaggy cap (Copc1, Copc2, Copc3, Copc5, Copc7); |
| | Rhodotorula mucilaginosa (Rhom1, Rhom2); Malassezia furfur |
| | (Malaf2, Malaf3, Malaf4); Malassezia sympodialis (Malas1, Malas5, |
| | Malas6, Malas7, Malas8, Malas9, Malas10, Malas11, Malas12, |
| | Malas13); Epicoccum purpurascens (Epip1); and Alternaria alternate |
| | (Alta1.0101, Alta1.0102), Aspergillus versicolor antigen, S. chartarum |
| | antigen), Cladosporium herbarum (Clah2, Clah5, Clah6, Clah7, Clah8, |
| | Clah9, Clah10, Clah12); Aspergillus flavus (Aspf113); |
| | animals (Bos domesticus dander allergen, Bos d 2, Bosd3, Bosd4, |
| | Bosd5, Bosd6, Bosd7, Bosd8, Bosd2.0101, Bosd2.0102, Bosd2.0103, |
| | Canis familiaris allergen, Can f 1, Canf2, Canf3, Canf4, Equus |
| | caballus allergen, Equc1, Equc2, Equc3, Equc4, Equc5, Felis |
| | domesticus allergen, Fel d 1, Feld2, Feld3, Feld4, Feld5w, Feld6w, |
| | Feld7w, guinea pig (Cavp1, Cavp2); Mouse Urinary Protein (MUP, |
| | Musm1) allergen, Mus m 1, Rat Urinary Protein (RUP, Ratn1) |
| | allergen, Rat n 1., Equus caballus (Equc2.0101, Equc2.0102)) |
| | Mosquito (Aeda1, Aeda2); honey bee (Apim1, Apim2, Apim4, |
| | Apim6, Apim7); bumble bee (Bomp1, Bomp4); German cockroach |
| | (Blag1, Blag2, Blag4, Blag5, Blag6, Blag7, Blag8); American |
| | cockroach (Pera1, Pera3, Pera6, Pera7); midge (Chit1-9, Chit1.01, |
| | Chit1.02, Chit2.0101, Chit2.0102, Chit3, Chit4, Chit5, Chit6.01, |
| | Chit6.02, Chit7, Chit8, Chit9); cat flea (Ctef1, Ctef2, Ctef3); pine |
| | processionary moth (Thap1); silverfish (Leps1); white face hornet |
| | (Dolm1, Dolm2, Dolm5); yellow hornet (Dola5); wasp (Pola1, Pola2, |
| | Pola5, Pole1, Pole5, Polf5, Polg5, Polm5, Vesvi5); Mediterranean |
| | paper wasp (Pold1, Pold4, Pold5); European hornet (Vespc1, Vespc5); |
| | giant asian hornet (Vespm1, Vespm5); yellowjacket (Vesf5, Vesg5, |
| | Vesm1, Vesm2, Vesm5, Vesp5, Vess5, Vesv1, Vesv2, Vesv5); |
| | Australian jumper ant (Myrp1, Myrp2); tropical fire ant (Solg2, |
| | Solg4); fire ant (Soli2, Soli3, Soli4); Brazilian fire ant (Sols2); |
| | California kissing bug (Triap1); Blattella germanica (Blag1.0101, |
| | Blag1.0102, Blag1.0103, Blag1.02, Blag6.0101, Blag6.0201, |
| | Blag6.0301); Periplaneta Americana (Pera1.0101, Pera1.0102, |
| | Pera1.0103, Pera1.0104, Pera1.02, Pera3.01, Pera3.0201, Pera3.0202, |
| | Pera3.0203, Pera7.0101, Pera7.0102); Vespa crabo (Vespc5.0101, |
| | Vespc5.0101); and Vespa mandarina (Vesp m 1.01, Vesp m 1.02) |
| | Nematode (Anis1, Anis2, Anis3, Anis4); pigeon tick (Argr1); worm |
| | (Ascs1); papaya (Carp1); soft coral (Denn1); rubber (latex)(Hevb1, |
| | Hevb2, Hevb3, Hevb4, Hevb5, Hevb6.01, Hevb6.02, Hevb6.03, |
| | Hevb7.01, Hevb7.02, Hevb8, Hevb9, Hevb10, Hevb11, Hevb12, |
| | Hevb13); human autoallergens (Homs1, Homs2, Homs3, Homs4, |
| | Homs5); obeche (Trips1); and Hevea brasiliensis (Hevb6.01, |
| | Hevb6.0201, Hevb6.0202, Hevb6.03, Hevb8.0101, Hevb8.0102, |
| | Hevb8.0201, Hevb8.0202, Hevb8.0203, Hevb8.0204, Hevb10.0101, |
| | Hevb10.0102, Hevb10.0103, Hevb11.0101, Hevb11.0102) |

### Foodstuff Testing

As summarized above, the present method may find use in analyzing a foodstuff sample, e.g., a sample from raw food, processed food, cooked food, drinking water, etc., for the presence of foodstuff markers. A foodstuff marker may be any suitable marker, such as those shown in Table 9, below, that can be captured by a capturing agent that specifically binds the foodstuff marker in a signal-amplifying nanosensor configured with the capturing agent. The environmental sample may be obtained from any suitable source, such as tap water, drinking water, prepared food, processed food or raw food, etc. In some embodiments, the presence or absence, or the quantitative level of the foodstuff marker in the sample may be indicative of the safety or harmfulness to a subject if the food stuff is consumed. In some embodiments, the foodstuff marker is a substance derived from a pathogenic or microbial organism that is indicative of the presence of the organism in the foodstuff from which the sample was obtained. In some embodiments, the foodstuff marker is a toxic or harmful substance if consumed by a subject. In some embodiments, the foodstuff marker is a bioactive compound that may unintentionally or unexpectedly alter the physiology if consumed by the subject. In some embodiments, the foodstuff marker is indicative of the manner in which the foodstuff was obtained (grown, procured, caught, harvested, processed, cooked, etc.). In some embodiments, the foodstuff marker is indicative of the nutritional content of the foodstuff. In some embodiments, the foodstuff marker is an allergen that may induce an allergic reaction if the foodstuff from which the sample is obtained is consumed by a subject.

In some embodiments, the present method further includes receiving or providing a report that indicates the safety or harmfulness for a subject to consume the food stuff from which the sample was obtained based on information including the measured level of the foodstuff marker. The information used to assess the safety of the foodstuff for consumption may include data other than the type and measured amount of the foodstuff marker. These other data may include any health condition associated with the consumer (allergies, pregnancy, chronic or acute diseases, current prescription medications, etc.).

The report may be generated by the device configured to read the signal-amplifying nanosensor, or may be generated at a remote location upon sending the data including the measured amount of the foodstuff marker. In some cases, a food safety expert may be at the remote location or have access to the data sent to the remote location, and may analyze or review the data to generate the report. The food safety expert may be a scientist or administrator at a governmental agency, such as the US Food and Drug Administration (FDA) or the CDC, a research institution, such as a university, or a private company. In certain embodiments, the food safety expert may send to the user instructions or recommendations based on the data transmitted by the device and/or analyzed at the remote location.

TABLE 9
Foodstuff Markers

| Source/Class | Marker/target |
|---|---|
| Pathogens/microbes | Bacillus anthracis (LF), Giardia lamblia, Legionella, Total |
| | Coliforms (including fecal coliform and E. Coli), Viruses |
| | (enteric) stapylococci (e.g., Staphylococcus epidermidis and |
| | Staphylococcus aureus (enterotoxin A, B, C, G, I, cells, TSST-1), |
| | Enterrococcus faecalis, Pseudomonas aeruginosa, Escherichia |
| | coli (Shiga-like toxin, F4, F5, H, K, O, bacteriophage K1, K5, |
| | K13), other gram-positive bacteria, and gram-negative bacilli. |
| | Clostridium difficile (Toxin A, B), Bacteroidetes, |
| | Cryptosporidium parvum (GP900, p68 or cryptopain, oocyst), |
| | Candida albicans, Bacillus anthracis, Bacillus |
| | stearothermophilus, Bacillus cereus, Bacillus licheniformis, |
| | Bacillus subtilis, Bacillus pumilus, Bacillus badius, Bacillus |
| | globigii, Salmonella typhimurium, Escherichia coli O157:H7, |
| | Norovirus, Listeria monocytogenes (internalin), Leptospira |
| | interrogans, Leptospira biflexa, Campylobacter jejuni, |
| | Campylobacter coli, Clostridium perfringens, Aspergillus flavus |
| | (aflatoxins), Aspergillus parasiticus (aflatoxins), Ebola virus |
| | (GP), Histoplasma capsulatum, Blastomyces dermatitidis (A |
| | antigen), Gram-positive bacteria (teichoic acid), Gram-ngative |
| | bacteria (such as Pseudomonas aeruginosa, Klebsiella |
| | pneumoniae, Salmonella enteriditis, Enterobacter aerogenes, |
| | Enterobacter hermanii, Yersinia enterocolitica and Shigella |
| | sonnei)(LPS), Polio virus, Influenza type A virus, Disease |
| | specific prion (PrP-d), Hepatitis A virus, Toxoplasma gondii, |
| | Vibrio cholera, Vibrio parahaemolyticus, Vibrio vulnificus, |
| | Enterococcus faecalis, Enterococcus faecium |
| Toxins/carcinogens | N-methylamino-L-alanine (BMAA), Clostridium botulinum |
| | neurotoxins, BoNT A, B, Ricin A, B; diphtheria toxin; |
| | Aristolochic acid; Colchicine, Ochratoxin A, Sterigmatocystin, |
| | Ergotamine, Fumonisins, Fusarin C, domoic acid, Brevetoxin, |
| | Mycotoxins |
| Halogenated | Heptachlor, chlordane |
| hydrocarbons | |
| Heavy metals | Lead, mercury, cadmium |
| Allergens | peanut (Ara h 1, Ara h 2, Ara h 6), fish, shellfish, mollusks, |
| | shrimp (D. pteronyssinus tropomyosin allergen, Der p 10) Cod |
| | (Gadc1); Atlantic salmon (Sals1); domestic cattle milk (Bosd4, |
| | Bosd5, Bosd6, Bosd7, Bosd8); chicken/egg (Gald1, Gald2, |
| | Gald3, Gald4, Gald5); shrimp (Mete1); shrimp (Pena1, Peni1); |
| | black tiger shrimp (Penm1, Penm2); squid (Todp1), brown |
| | garden snail (Helas1); abalone (Halm1); edible frog (Rane1, |
| | Rane2); oriental mustard (Braj1); rapeseed (Bran1); cabbage |
| | (Brao3); turnip (Brar1, Brar2); barley (Horv15, Horv16, Horv17, |
| | Horv21); rye (Secc20); wheat (Tria18, Tria19, Tria25, Tria26, |
| | gliadin); corn (Zeam14, Zeam25); rice (Orys1), celery (Apig1, |
| | Apig4, Apig5); carrot (Dauc1, Dauc4); hazelnut (Cora1.04, |
| | Cora2, Cora8); strawberry (Fraa1, Fraa3, Fraa4); apple (Mald1, |
| | Mald2, Mald3, Mald4); pear (Pyrc1, Pyrc4, Pyrc5); avocado |
| | (Persa1); apricot (Pruar1, Pruar3); sweet cherry (Pruav1, Pruav2, |
| | Pruav3, Pruav4); European plum (Prud3); almond (Prudu4); |
| | peach (Prup3, Prup4); asparagus (Aspao1); saffron crocus |
| | (Cros1, Cros2); lettuce (Lacs1); grape (Vitv1); banana (Musxp1); |
| | pineapple (Anac1, Anac2); lemon (Cit13); sweet orange (Cits1, |
| | Cits2, Cits3); litchi (Litc1); yellow mustard (Sinai); soybean |
| | (Glym1, Glym2, Glym3, Glym4); mung bean (Vigr1); peanut |
| | (Arah1, Arah2, Arah3, Arah4, Arah5, Arah6, Arah7, Arah8); |
| | lentil (Lend, Lenc2); pea (Piss1, Piss2); kiwi (Actc1, Actc2); |
| | bell pepper (Capa1w, Capa2); tomato (Lyce1, Lyce2, Lyce3); |
| | potato (Solat1, Solat2, Solat3, Solat4); Brazil nut (Bere1, Bere2); |
| | black walnut (Jugn1, Jugn2); English walnut (Jugr1, Jugr2, |
| | Jugr3); Cashew (Anao1, Anao2, Anao3); Castor bean (Ricc1); |
| | sesame (Sesi1, Sesi2, Sesi3, Sesi4, Sesi5, Sesi6); muskmelon |
| | (Cucm1, Cucm2, Cucm3); Chinese-date (Zizm1); Anacardium |
| | occidentale (Anao1.0101, Anao1.0102); Apium graveolens |
| | (Apig1.0101, Apig1.0201); Daucus carota (Dauc1.0101, |
| | Dauc1.0102, Dauc1.0103, Dauc1.0104, Dauc1.0105, |
| | Dauc1.0201); Citrus sinensis (Cits3.0101, Cits3.0102); Glycine |
| | max (Glym1.0101, Glym1.0102, Glym3.0101, Glym3.0102); |
| | Lens culinaris (Lenc1.0101, Lenc1.0102, Lenc1.0103); Pisum |
| | sativum (Piss1.0101, Piss1.0102); Lycopersicon esculentum |
| | (Lyce2.0101, Lyce2.0102); Fragaria ananassa (Fraa3.0101, |
| | Fraa3.0102, Fraa3.0201, Fraa3.0202, Fraa3.0203, Fraa3.0204, |
| | Fraa3.0301); Malus domestica (Mald1.0101, Mald1.0102, |
| | Mald1.0103, Mald1.0104, Mald1.0105, Mald1.0106, |
| | Mald1.0107, Mald1.0108, Mald1.0109, Mald1.0201, |
| | Mald1.0202, Mald1.0203, Mald1.0204, Mald1.0205, |
| | Mald1.0206, Mald1.0207, Mald1.0208, Mald1.0301, |
| | Mald1.0302, Mald1.0303, Mald1.0304, Mald1.0401, |
| | Mald1.0402, Mald1.0403, Mald3.0101w, Mald3.0102w, |
| | Mald3.0201w, Mald3.0202w, Mald3.0203w, Mald4.0101, |
| | Mald4.0102, Mald4.0201, Mald4.0202, Mald4.0301, |
| | Mald4.0302); Prunus avium (Pruav1.0101, Pruav1.0201, |
| | Pruav1.0202, Pruav1.0203); and Prunus persica (Prup4.0101, |
| | Prup4.0201) |
| Synthetic hormone | 17beta-estradiol (E2), estrone (El), estrogen (ES: El + E2 + |
| analogues | estradiol (E3)), 1 7alfa-ethynylestradiol (EE2), 4-nonylphenpol, |
| | testosterone, Diethylstilbestrol (DES), recombinant bovine |
| | growth hormone (rBGH) |
| Pesticides | Dieldrin, carbaryl, chlorpyrifos, parathion, aldrin, endosulfan I, |
| | endrin, toxaphene, O-ethyl O-4-nitrophenyl phenylphosphono- |
| | thioate |
| | (EPN), fenitrothion, pirimiphos-methyl, thiabendazole, |
| | methiocarb, Carbendazim, deltamethrin, Avermectin, Carbaryl, |
| | Cyanazine, Kresoxim, resmethrin, kadethrin, cyhalothrin, |
| | biphenthrin, fenpropathrin, allethrin and tralomethrin; aromatic- |
| | substituted alkanecarboxylic acid esters such as fenvarerate, |
| | flucythrinate, fluvalinate and cycloprothrin; and non-ester |
| | compounds such as etofenprox, halfenprox (MTI-732), 1-(3- |
| | phenoxyphenyl)-4-(4-ethoxyphenyl)-4-methylpentane (MTI- |
| | 790), 1-(3-phenoxy-4-fluorophenyl)-4-(4-ethoxyphenyl)-4- |
| | methylpentane (MTI-800), dimethyl-(4-ethoxyphenyl)-(3- |
| | phenoxybenzyloxy)silane (SSI-116), silafluofen and PP-682, |
| | carbofuran, triazophos |
| Herbicide | atrazine, deethylatrazine, cyanazine, terbuthylazine, terbutryn, |
| | molinate, simazine, prometon, promteryn, hydroxyatrazine, 2,6- |
| | dichlorobenzamide (BAM), N-dealkylated triazines, mecoprop, |
| | thiram, acetochlor, alachlor, Chlorothalonil, Chlorsulfuron, |
| | Fenoxaprop ethyl, Linuron, monuron, diuron, Quizalofop-ethyl, |
| | Imazalil, Iprodione, Iprovalicarb, Myclobutanil |
| Industrial | Dioxin (2,3,7,8-TCDD), 4-tert-octylphenol, bisphenol A (BPA), |
| material/waste | Styrene, Di(2-ethylhexyl) phthalate, Dibutyl phthalate (DBP), |
| | benzophenone, benzene, trichloroethylene, polychlorinated |
| | biphenyl (PCB), nonylphenol, p-cresol, melamine, xylene |
| Antibiotics | 3-Amino-5-morpholinomethyl-2-oxazolidone (AMOZ; tissue |
| | bound metabolite of furaltadone), oxytetracycline, |
| | rolitetracycline, Actinomycin D, Amikacin sulfate, |
| | Aminoglycosides, nitrofuran (AOZ), Chloramphenicol, |
| | Doxycycline, Streptomycin, gentamicin, neomycin, kanamycin, |
| | sulfamethazine, enrofloxacin, sulfadiazine, enrofloxacin |
| Food coloring/ | Tartrazine, ethoxyquin, erythritol, penicillin, Fluoroquinolone, |
| additive/ | Malachite Green/Leucomalachite Green, C.I. Solvent Yellow 14 |
| preservative | (Sudan I), |
| Food preparation | Acrylamide, 2-amino-3-methylimidazo(4,5-f)quinolone, |
| | Benzo[a]pyrene |
| Nutritional content | Vitamins A (retinol), B12 (cobalmins), B6 (pyridoxine), B1 |
| | (thiamin), B2 (riboflavin), B3 (niacin), B5 (D-pantothenic acid), |
| | B7 (biotin), B9 (folic acid), C, D, E (alpha-tocopherol); |
| Other | Caffeine, Ovine myofibril proteins, Etodolac |

### Kits

Aspects of the present disclosure include a kit that find use in performing the present method, as described above. In certain embodiments the kit includes a signal-amplifying nanosensor configured to specifically bind an analyte, e.g., an analyte selected from Tables 1, 2, 3, 7, 8, or 9, or an antibody analyte that binds specifically to an epitope listed in Tables 4, 5 and 6. In certain embodiments, the kit includes instructions for practicing the subject methods using a hand held device, e.g., a mobile phone. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Another means would be a computer readable medium, e.g., diskette, CD, DVD, Blu-Ray, computer-readable memory, etc., on which the information has been recorded or stored. Yet another means that may be present is a website address which may be used via the Internet to access the information at a removed site. The kit may further include a software for implementing a method for measuring an analyte on a device, as described herein, provided on a computer readable medium. Any convenient means may be present in the kits.

In some embodiments, the kit includes a detection agent that includes a detectable label, e.g. a fluorescently labeled antibody or oligonucleotide that binds specifically to an analyte of interest, for use in labeling the analyte of interest. The detection agent may be provided in a separate container as the signal-amplifying nanosensor, or may be provided in the signal-amplifying nanosensor.

In some embodiments, the kit includes a control sample that includes a known detectable amount of an analyte that is to be detected in the sample. The control sample may be provided in a container, and may be in solution at a known concentration, or may be provided in dry form, e.g., lyophilized or freeze dried. The kit may also include buffers for use in dissolving the control sample, if it is provided in dry form.

### EXEMPLARY EMBODIMENTS

### Example 1: Ultra-Sensitive, Rapid, Fluorescence Assay Platform for Disease/Cancer Early Diagnosis and Personalized Medicine

1. Overview. An assay platform, disk-coupled dots-on-pillar antenna array (D2PA)-Assay, that has demonstrated the detection of biomarkers (proteins or DNAs) with a sensitivity of 4-6 orders of magnitude higher than the existing best commercial technology has been developed. The developed assay platform can be broadly applied to sensitivity enhancement of nearly all fluorescence/luminescence based assays, and is fast, simple-to-use, and low cost. Already, it has demonstrated such sensitivity enhancement in detecting the biomarkers of Alzheimer's disease (AD), prostate cancers and breast cancer. The ultrasensitive assay platform also has enormous applications in other areas in human healthcare (allergy, food safety, etc) and other bio/chemical sensing areas (animal, agriculture, bio-threat detections, etc.)

2. Technology. Protein and DNA detection is universal and vital in biological study and medical diagnosis. Fluorescent assay (immuno or DNA), which identifies a targeted protein or DNA biomarker (i.e., analyte) by selectively tagging it with a detection agent (antibody or detecting DNA) labeled with fluorophores, is one of the most widely used and most sensitive methods. When excited by light, the fluorophore's fluorescent intensity is related to the existence and the concentration of the biomarker.

Fluorescence can be enhanced by metallic nanostructures through light focusing. The developed assay platform uses a special nanostructure surface, termed “disk-coupled dots-on-pillar antenna array” (D2PA), that couples subwavelength-size small metallic nanoparticles for focusing light with wavelength-size 3D antennas for good light absorption and radiation, drastically enhancing fluorescence for a given excitation power and hence fluorophore detection sensitivity (3 to 5 orders of magnitude). One example of the D2PA consists of a periodic dielectric pillar array (200 nm pitch and ˜100 nm diameter), a metallic disk (˜135 nm diameter) on top of each pillar, a metallic backplane on the foot of the pillars, subwavelength metallic nanodots randomly located on the pillar walls, and nanogaps between these metal components (FIG. 3). The metallic disk and the metallic back plane form a 3D cavity antenna.

FIG. 3. Immuno or DNA assay platform (D2PA assay) and Beta-amyloid (AR) Immunoassay. (a) Schematic. D2PA assay plate at the bottom of a standard 96 well plate. (b) Zoomed-in. (c) Schematic, (d) top view and (e) close-up of scanning electron micrograph of the D2PA. And (f) Schematic of a fluorescent sandwich immunoassay placed on the bio-functioned D2PA plate (the coupling layer is DSU and Protein A)

Furthermore, technologies that can place the biomarkers at “hot-spots” (the highest enhancement locations), whereby these developed technologies further increase detection sensitivity by another 10 to 100 fold (so total 4 to 6 orders of magnitude), and technologies that can manufacture such structures uniformly, in large volume, and low cost, were developed.

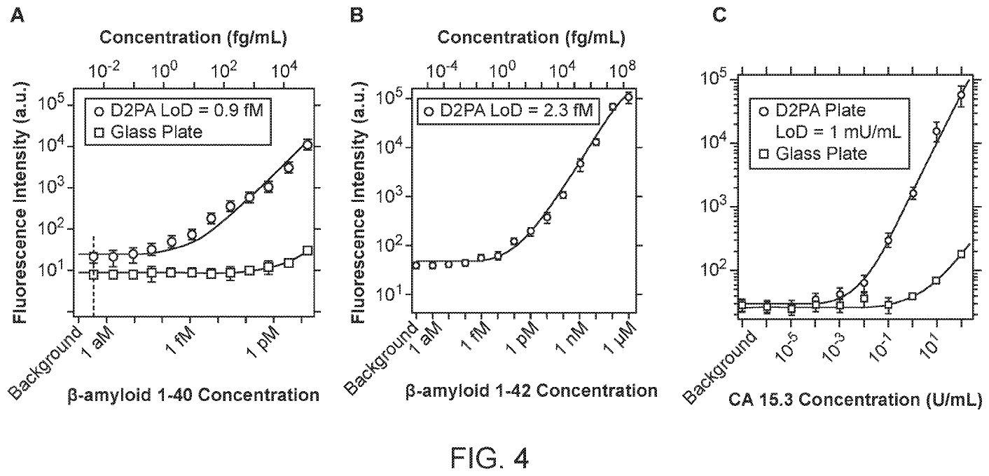

To form a biomarker assay, a coupling agent layer was coated on top of D2PA and then capture agent. After having captured the targeted biomarkers by the capture agent, labeled detection agent were used to selectively bond and identify the captured biomarker. For a given biomarker, a selective pair of capture and detection agents is used. Since the fluorescence enhancement in D2PA-Assay does not modify assay chemistry but only light radiation physics, such fluorescence enhancement can be broadly applied to all existing fluorescence assays. For example, in the detecting AD biomarker, Aα-42/40, commercial “Aβ-42/40 ELISA kits” (Covance USA) were purchased, where the enzyme and the substrate were not used, but rather commercial streptavidinconjugated fluorescence (IRDye800CW) labels (Rockland USA) were attached to the detection agent. The rest of the kit was used as provided by the manufacturer. Similar assays on D2PA plate for detection of prostate specific antigen (PSA), and CA15.3 cancer and carcinoembryonic antigen (CEA) biomarkers were also implemented (FIG. 4).

FIG. 4 Immunoassay standard curves for different biomarkers on D2PA. (a) Measured fluorescence response of A040 standard on D2PA plate (circle) and glass plate (square). LoD=0.2 fM (D2PA) and 10 pM (glass), respectively (50,000 enhancement). (b) Aβ 42 LoD=2.3 fM with a broad dynamic range of 6 orders of magnitude. (c) CA15.3 LoD=0.001 U/mL for D2PA plate and 5 U/mL for glass plate. (5,000×). An ultra-sensitive assay of the present disclosure allows (a) discovery of new biomarkers, (b) detection of a known biomarker in a different body fluid, where biomarker concentration much lower but sampling is much easier (noninvasively) (e.g. replace cerebrospinal fluid (CSF) sampling by saliva); and (c) diagnosis a test using smart phone rather than fancy ultra-high resolution reader.

3. Noninvasive early detection of Alzheimer's disease (AD). The concentrations of beta-amyloid (Aβ)-42 and tau in cerebrospinal fluid (CSF) are key biomarkers to diagnosis AD. However, the procedure for extracting CSF is very aggressive, requires specially trained professionals, has certain risks, and produce only a very small amount of CSF each time. Thus it would be advantageous to measure A3-42 concentration in saliva for AD diagnosis. The D2PA Aβ-42 assay has a LoD of 2.3 fg/mL (basic model) and 92 ag/mL (advanced model), which are ˜ 500 and 11,000 fold higher than previous methods.

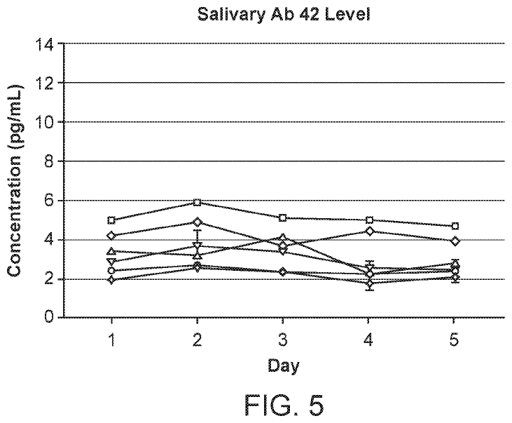

Using D2PA assay, the Aβ-42 concentration in saliva of 6 healthy males (all volunteers) in five consecutive days was measured (FIG. 5). The measured Aβ-42 concentrations were very consistent and stable in saliva, indicating the Aβ-42 in saliva is a good marker in AD study.

FIG. 5. 5-consecutive-day monitoring of salivary Beta Amyloid 1-42 level from 6 healthy human subjects. morning. The average 5-day variance of the subjects are 13.3%.

The following steps are proposed: (a) expand the size of saliva testing pool (having different genders, age variations, life style variation, etc), (b) expand the AD biomarkers tested beyond Aβ-42 (tau, ApoE, BNP, etc) for better diagnosis accuracy, and (c) in collaboration with National Alzheimer's disease Centers, get the saliva from the AD patients, test AD biomarkers using D2PA assay, and compare with their CSF test and clinical tests. These studied will provide solid evidence if the Aβ-42 and other protein markers in saliva can be used in early detection of AD.

4. Noninvasive Early detection of breast cancer. CA15.3 is a tumor marker associated with mammary tumors. Increased levels of CA15.3 in serum have been observed in patients with breast cancer. It has been clinically approved to use CA15.3 for the monitoring, prognosis, and early detection of cancer recurrence. High elevated level of CA15.3, can provide valuable information for the early detection of the disease. Use of saliva is much simpler than serum and can be administrated by patients themselves. Compared with <30 U/mL in serum, CA15.3 in saliva for healthy human is <5 U/mL. Using the D2PA assay, the LoD was 0.001 U/mL, 5,000× more sensitive than previous assays, which is more than sufficient to identify CA15.3 in saliva. The use of the D2PA assay in measuring CA15.3 in healthy human will be investigated to validate CA15.3 in saliva, and then test CA15.3 in the saliva from cancer patients, and compare with other tests to validate D2PA in cancer early diagnosis.

5. Smart-phone based diagnosis assays for personalized medicine. The hardware and software for reading an assay using a smart phone will be developed, and the limit of detection (LoD) allowed by such approach will be determined (FIG. 1: Smart-phone based detection of fluorescence immunoassay on D2PA chips). The present ultra-sensitive assay platform technology will allow many diseases/cancer and other health related tests to be performed by smart-phone. In hardware, dipstick (self-pumping and multiplexed agents) will be designed and fabricated, and LED lighting and filters will be added. Software to control the reading and data analysis will be written. Initially simple fluorophors will be used in the test.

6. Further improve the assay technology, particularly even higher sensitivity and faster speed. The D2PA sensitivity, precision, linearity and repeatability will be improved by (i) optimizing the design of the D2PA (e.g. nanopillar size, pillar heights, nanodot size, nanogaps, metal used, other coupling layer) and (ii) using different fluorescence measurement methods (e.g. area-integrated measurement vs. pixel counting).

### Example 2: Smartphone-Based Assay Platform for Low-Cost, Rapid, Point-of-Care, Fetal/Infant Brain Function and Damage Diagnostics

An exemplary implementation is described of a method that enhances the sensitivity of an existing assay over one million fold (i.e. 106) and will allow low-cost, rapid, point-of-care assays for diagnosing fetal/infant brain function or damages that can be read by a smartphone (rather than a high-sensitivity, expensive, professional-operated, reader) and performed by an ordinary person.

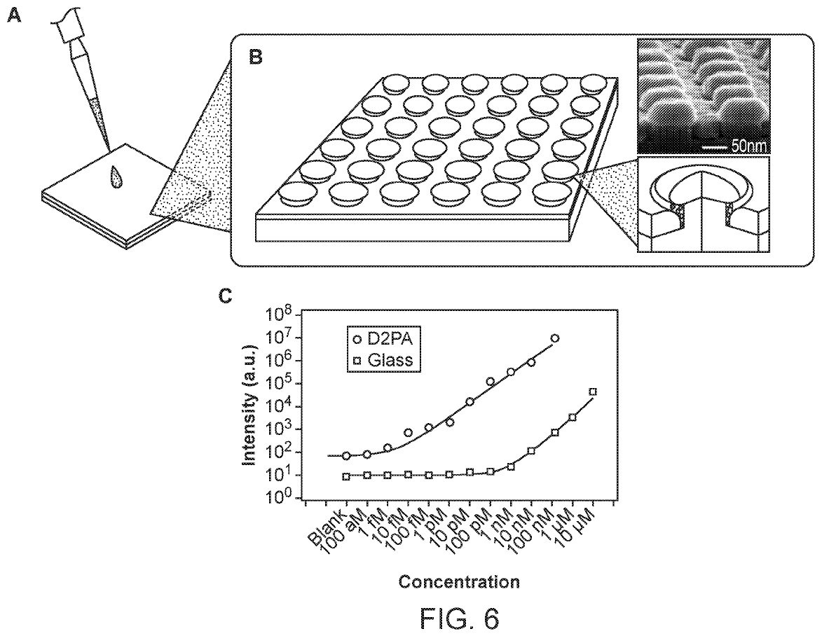

A method of amplifying the fluorescent signal on an assay plate, having demonstrated a signal amplification of over one million fold (from 0.9 nM to 300 aM) and a dynamic range over seven orders of magnitude, compare to the same assay on a glass plate and read by the same reader has been developed (FIG. 6. Ig G assay).

FIG. 6. Schematic of a nanoplasmonic-enhanced immunoassay plate, termed D2PA (disk-coupled dots-on-pillar antenna-array) (a) and the nanostructured surface (b), where the D2PA enhances an immuo- or DNA fluorescent assay sensitivity by over one million fold (e.g. IgG direct assay from 0.9 nM on glass plate to 300 aM on D2PA (c))

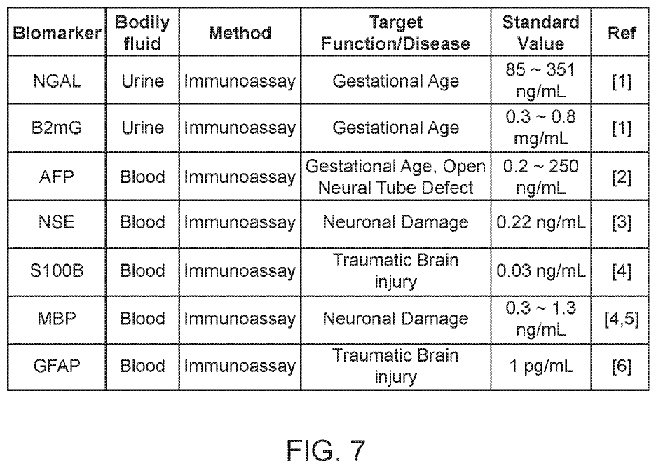

This high sensitivity enhancement on the assay plate removes the need for a high-sensitivity assay reader, and allows an assay reading by a smart-phone operated by an ordinary person. The smartphones displays the instructions to patient and transmits the assay data to doctors (FIG. 9). A detection sensitivity of 2 pg/mL (13.8 fM) has been demonstrated by using an equivalent smartphone camera as the assay reader, which is 1,000 times lower than using high sensitive lab-grade reader but still 1,000 times higher than the sensitivity using an conventional assay plate and a high sensitive lab-grade reader. Such smartphone sensitivity is sufficient for reading most brain biomarkers, which are in ˜ ng/mL range (See table in FIG. 7).

Since this method amplifies the fluorescent signal on an assay plate by a physical process (nanoplasmonic effects), rather than traditional bio/chemical amplification, it can be used to enhance all existing fluorescence assays (virtually no new bio/chemistry development required).

The method achieves the high sensitivity by solving three key problems in conventional fluorescence assay: (i) low absorption of excitation light, (ii) low fluorophor quantum efficiency, and (iii) low far field emission by the fluorophore. The special nanostructures (D2PA (disk-coupled dots-on-pillar antenna-array)) that were designed provide ˜2000×, ˜10× and ˜50× enhancement for each factor, respectively, leading to a total ˜1,000,000 enhancement. The D2PA has an enhancement factor of 100× to 1,000× higher than other existing plasmonic nanstructures (e.g. gold nanoparticles), because the D2PA has a special structure to solve the conflicting size requirements. The D2PA is also low-cost due to its simple structure.

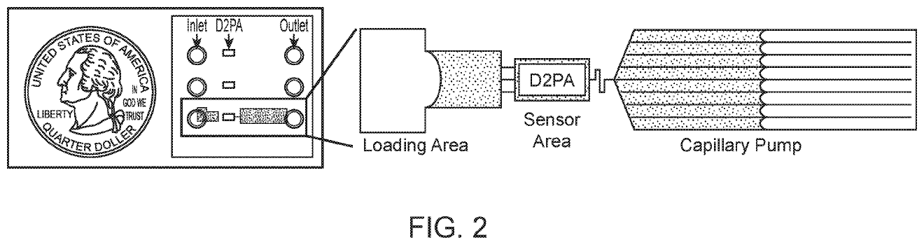

A complete assay card (˜1 cm by 1 cm area and <1 mm thick) will be developed, where a patient is merely required to drop a droplet of blood or urine (˜10 μL), wait a few minutes (<5 min), and take a picture by a smartphone to read test results. The complete assay card has passive-pumps, microfluidic channels, filters, and pre-coated detection reagents (which may include a labeling agent) thus no extra chemical loading or plug-in power is required during operation (FIG. 2). The feasibility of the present technology in smartphone based assay will be demonstrated.

Existing D2PA plates and the reagents (capture and detection agents) will be used for several common biomarkers for brain function and damage from commercial vendors to form the assay, and then different grades of smartphones will be used as the reader to characterize the assay sensitivity and other parameters (FIGS. 6, 7 and 8). The standard curves will be measured first and the spike and recovery to simulate the real samples will be measured.

FIG. 8. Schematic of the testing sequence in the proposed project. (a) prepare the D2PA plate, (b) immobilizing capture agents, (c) catch and label the target biomarker, and (d) read by different types of mobile-phones to see how the detection sensitivity and accuracy depends on the phone (for a given biomarker). (Note, biomarkers and reagents are from commercial vendors).

In feasibility tests for assessing gestational age, assays for neutrophil gelatinase-associated lipocalin (NGAL) and beta-2-microglobulin (B2mG) in urine, and Alpha-fetoprotein (AFP) in maternal blood will be created and tested. Urine NGAL and B2mG were found to vary by gestational age because they are related to the infants' kidney development, which closely correlate with different gestational age. AFP level has been widely recognized to be highly correlated with gestational stage, whose concentration range from 0.2 ng/mL for non-pregnant women to 250 ng/mL for pregnant women at 32 weeks.

For the diagnosis of brain injury/function, assays for neuron-specific enolase (NSE), S100B, myelin basic protein (MBP) and glial fibrillary acidic protein (GFAP) in blood will be created and tested. These molecules are released from brain neuron into cerebrospinal fluid (CSF) after brain injury and some of them passed into blood. Other brain function biomarkers can be implemented into this smartphone platform.

The D2PA plate can be mass produced at low cost, and has fast assay time due to much reduced diffusion length provided by the microfluidic channels. A low-cost D2PA plate fabrication involves only two steps: one step of patterning the nanostructures and microfluidic channels, which can be done in one step of nanoimprint; and one step of a thin metal deposition. Since the gold is so thin (40 nm thick), the cost of the gold is less than 0.4 cent per 1 cm by 1 cm D2PA tester. The entire chip is expected to cost less than 10 cents (USD) in mass production.

Feasibility demonstration of the present smartphone-based assay platform that can measure all the proposed biomarkers using small droplet of blood or urine samples (˜10 μL) within 5 minutes and achieve a diagnostic accuracy >90% will be demonstrated.

A complete integrated assay card (with passive-pumps, microfluidic channels, filters, and pre-coated biochemical reagents) ready for field use with a smartphone (i.e. the patient just need to drop a body fluid and take a picture) will be developed. Technologies for integration, scale-up, low-cost D2PA plate manufacturing will be developed. Software construction for mobile triage function using cloud-based diagnosis information communication will be developed.

### Example 3: Smart-Phone Based Personalized Medicine

With reference to FIG. 2, an exemplary method, according to an embodiment of the present disclosure, is shown below.
1. Having signal-amplifying nanosensor
2. Put a droplet of sample (saliva, blood, sweet, urine, feats, . . . ) on the signal-amplifying nanosensor chip.
3. Reading the chip by smartphone
4. Smartphone displays: normal, attention, warning, caution, emergency, (see FIG. 2 for details)
5. Test info being transmitted to data base, physician, hospital, etc. (see FIG. 2 for details)
6. Instructions being transmitted back.
7. Person takes actions to do X.
8. The use of above test are: (a) daily health test, (b) disease/cancer monitoring, (c) patient off-hospital monitoring, (d) allergy, . . . .

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An analyte measurement method, comprising:
a) obtaining a sample;
b) applying the sample to a signal-amplifying nanosensor, comprising:
(i) a substrate;
(ii) a signal amplification layer; and
(iii) a capture agent that specifically binds to an analyte in the sample,
wherein the capture agent is linked to the surface of the signal amplification layer and said nanosensor amplifies a light signal from labeled analytes that are bound to the signal amplification layer via the capture agent, under conditions suitable for binding of the analyte in a sample to the capture agent;
c) washing the signal-amplifying nanosensor; and
d) reading the signal-amplifying nanosensor, thereby obtaining a measurement of the amount of the analyte in the sample.

2. The method according to claim 1, wherein the sample is a liquid sample.

3. The method according to claim 1, wherein the applying step b) comprises applying a sample to a microfluidic device comprising the signal-amplifying nanosensor.

4. The method according to claim 1, wherein the reading step d) comprises detecting a fluorescence or luminescence signal from the signal-amplifying nanosensor.

5. The method according to claim 1, wherein the reading step d) comprises reading the signal-amplifying nanosensor with a handheld device configured to read the signal-amplifying nanosensor.

6. The method according to claim 5, wherein the handheld device is a mobile phone.

7. The method according to claim 1, wherein the signal-amplifying nanosensor comprises a labeling agent that can bind to an analyte-capture agent complex on the signal-amplifying nanosensor.

8. The method according to claim 1, wherein the method comprises between steps c) and d):
applying to the signal-amplifying nanosensor a labeling agent that binds to an analyte-capture agent complex on the signal-amplifying nanosensor; and
washing the signal-amplifying nanosensor.

9. The method according to claim 1, wherein the reading step d) comprises reading an identifier for the signal-amplifying nanosensor.

10. The method according to claim 9, wherein the identifier is an optical barcode, a radio frequency ID tag, or combinations thereof.

11. The method according to claim 1, wherein the method further comprises:
applying a control sample to a control signal-amplifying nanosensor comprising a capture agent that binds to the analyte, wherein the control sample comprises a known detectable amount of the analyte; and
reading the control signal-amplifying nanosensor, thereby obtaining a control measurement for the known detectable amount of the analyte in a sample.

12. The method according to claim 1, wherein the sample is a diagnostic sample obtained from a subject, the analyte is a biomarker, and wherein the amount of the analyte in the sample is diagnostic of a disease or a condition.

13. The method according to claim 12, wherein the sample is saliva, serum, blood, sputum, urine, sweat, lacrima, semen, or mucus.

14. The method according to claim 12, wherein the method further comprises:
receiving a report that indicates:
the measured amount of the biomarker; and
a range of measured values for the biomarker in an individual free of or at low risk of having the disease or condition,
wherein the measured amount of the biomarker relative to the range of measured values is diagnostic of a disease or condition.

15. The method according to claim 12, wherein the method further comprises:
providing to the subject a report that indicates:
the measured amount of the biomarker; and
a range of measured values for the biomarker in an individual free of or at low risk of having the disease or condition,
wherein the measured amount of the biomarker relative to the range of measured values is diagnostic of a disease or condition.

16. The method according to claim 12, wherein the method further comprises:
diagnosing the subject based on information comprising the measured amount of the biomarker in the sample.

17. The method according to claim 16, wherein the diagnosing step comprises sending data comprising the measured amount of the biomarker to a remote location and receiving a diagnosis based on information comprising the measurement from the remote location.

18. The method according to claim 12, wherein the biomarker is selected from Tables 1, 2, 3 or 7.

19. The method according to claim 18, wherein the biomarker is a protein selected from Tables 1, 2, or 3.

20. The method according to claim 18, wherein the biomarker is a nucleic acid selected from Tables 2, 3 or 7.

21-43. (canceled)

44. A kit comprising:
a signal-amplifying nanosensor comprising a capture agent that binds to an analyte of interest in a sample; and
instructions for reading the signal-amplifying nanosensor, thereby obtaining a measurement of the amount of the analyte in the sample.

45-53. (canceled)
